(19) United States
(12) Patent Application Publication
(10) Pub. No.: US 2025/0161420 A1
(43) Pub. Date: May 22, 2025

(54) PEPTIDE CONJUGATE VACCINE COMPOSITIONS AND METHODS FOR THE TREATMENT OF ALZHEIMER'S DISEASE

(71) Applicant: Merck Sharp & Dohme LLC, Rahway, NJ (US)

(72) Inventors: Yen-Pang Hsu, Saugus, MA (US); Sampat L. Ingale, Lexington, MA (US); Izzat T. Raheem, Doylestown, PA (US); Jeffrey William Schubert, North Wales, PA (US); Ryan P. Staupe, Havertown, PA (US); Chengwei Wu, Maple Glen, PA (US)

(73) Assignee: Merck Sharp & Dohme LLC, Rahway, NJ (US)

(21) Appl. No.: 18/947,508

(22) Filed: Nov. 14, 2024

Related U.S. Application Data
(60) Provisional application No. 63/599,885, filed on Nov. 16, 2023.

(51) Int. Cl. A61K 39/00; A61K 39/385; A61K 47/64

(52) U.S. Cl. CPC A61K 39/0007; A61K 39/385; A61K 47/646; A61K 2039/5258; A61K 2039/55505; A61K 2039/55555; A61K 2039/55561; A61K 2039/55577; A61K 2039/6037; A61K 2039/6068; A61K 2039/6081; A61K 2039/64

(57) ABSTRACT

The invention provides compositions and methods for the treatment of diseases associated with amyloid deposits of Aβ in the brain of a patient, such as Alzheimer's Disease. Such methods entail administering a pharmaceutical composition comprising an immunogenic fragment of Aβ capable of inducing a beneficial immune response in the form of antibodies to Aβ. The immunogenic fragments comprise linear or multivalent peptides of Aβ. Pharmaceutical compositions comprise the immunogenic fragment chemically linked to a carrier molecule which may be administered with an adjuvant.

### CROSS-REFERENCE TO RELATED APPLICATIONS

This application claims the benefit of U.S. Provisional Patent Application Ser. No. 63/599,885 filed Nov. 16, 2023, the entire contents of which are incorporated by reference herein.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 1, 2024, is named 25864-WO-PCT_SL.xml and is 59,964 bytes in size.

### FIELD

This disclosure relates generally to peptide-based pharmaceutical compositions and conjugate vaccines, as well as methods of using the compositions and vaccines for the prevention and treatment of neurodegenerative diseases, such as Alzheimer's Disease.

### BACKGROUND

Alzheimer's Disease (AD) is a progressive neurodegenerative disease characterized by dementia and pathological changes to the brain, including accumulation of amyloid plaques and neurofibrillary tangles. J. A. Hardy, et al., Alzheimer's Disease: the amyloid cascade hypothesis. Science (1992) 256:184-185. According to the “amyloid cascade hypothesis” increased accumulation of amyloid β (Aβ) peptide in the brain leads to formation of Aβ oligomers and plaques that lead to neurite injury, formation of neurofibrillary tangles, neuronal cell death, and cognitive decline. J. A. Hardy, et al., The amyloid hypothesis of Alzheimer's Disease: progress and problems on the road to therapeutics. Science (2002) 297:353-356.

Aβ peptides consist of 40 to 42 amino acid sequences derived from the amyloid precursor protein (APP). An N-terminally truncated version, Aβ_{pE3}, results from cleavage of two N-terminal amino acids from Aβ₁₋₄₂ and cyclization of the glutamic acid sidechain to form pyroglutamate. T. Iwatsubo, et al., Full-length amyloid-beta (1-42(43)) and amino-terminally modified and truncated amyloid-beta 42(43) deposit in diffuse plaques. Am J Pathol. (1996) 149(6):1823-30. Aβ_{pE3} peptides are highly prone to aggregation and accumulate early in the plaque forming amyloid cascade. T. C. Saido, et al., Dominant and differential deposition of distinct beta-amyloid peptide species, Aβ N3(pE), in senile plaques. Neuron (1995)14:457-466. T. Iwatsubo, et al., Full-length amyloid-beta (1-42(43)) and amino-terminally modified and truncated amyloid-beta 42(43) deposit in diffuse plaques. Am. J. Pathol. (1996) 149:1823-1830. Y. M. Kuo, et al., Isolation, chemical characterization, and quantitation of A3 3-pyroglutamyl peptide from neuritic plaques and vascular amyloid deposits. Biochem. Biophys. Res. Commun., (1997) 237:188-191. S. Schilling, et al., On the seeding and oligomerization of pGlu-amyloid peptides (in vitro). Biochemistry (2006) 45:12393-12399. Z. Schlenzig, et al., Pyroglutamate formation influences solubility and amyloidogenicity of amyloid peptides. Biochemistry (2009) 48:7072-7078.

Clearance of Aβ plaques has been postulated as a means for slowing progression of AD, and passive immunotherapeutic approaches targeting Aβ_{pE3} have demonstrated plaque clearance and slowing of cognitive decline in clinical trials. J. R. Sims, et al., Donanemab in early symptomatic Alzheimer Disease: the TRAILBLAZER-ALZ 2 randomized clinical trial. JAMA. (2023) 330(6):512-527. Active immunotherapy (i.e., vaccine) therapies have also been tested for the treatment of Alzheimer Disease. F. Mantile, et al., Vaccination against 0-amyloid as a strategy for the prevention of Alzheimer's Disease. Biology (Basel). (2020) 9(12):425. Vaccine candidate AN1792, consisting of Aβ₁₋₄₂ with QS-21 adjuvant, was tested in a Phase II clinical trial and showed evidence of plaque lowering post-mortem. Nevertheless, AN1792 induced meningoencephalitis, attributed to T cell responses to the vaccine, in a subset of patients. J. A. R. Nicoll, et al., Persistent neuropathological effects 14 years following amyloid-O immunization in Alzheimer's Disease. Brain (2019) 142(7):2113-2126.

At present, there is no specific and highly effective prophylactic or therapeutic treatment for Alzheimer's Disease. Hence, there a strong need for additional therapeutic measures.

### SUMMARY

The object of the present invention is to provide pharmaceutical compositions for the prophylactic and therapeutic treatment of Alzheimer's Disease. In particular, aspects of the present invention provide for Alzheimer's Disease vaccines which may be administered to a mammal for the prevention and/or treatment of Alzheimer's Disease.

The present disclosure provides compositions and methods for the treatment of diseases, such as Alzheimer's Disease, which are associated with amyloid deposits of Aβ in the brain of a patient. In one aspect, the present disclosure provides a pharmaceutical composition comprising an immunogenic peptide of at least six contiguous amino acids of SEQ ID NO:1 and an immunogenic carrier protein.

In some embodiments, the immunogenic peptide lacks 1-10 amino acids at the N-terminus or 1-33 amino acids at the C-terminus of SEQ ID NO:1. In other embodiments, the immunogenic peptide lacks 1-10 amino acids at the N-terminus and 1-33 amino acids at the C-terminus of SEQ ID NO:1.

In some embodiments, the immunogenic peptide comprises at least ten contiguous amino acids of SEQ ID NO:1.

In some embodiments, the immunogenic peptide comprises 1-5 modified amino acids. In certain embodiments, the immunogenic peptide comprises a pyroglutamate residue at amino acid position 3 and/or 11 of SEQ ID NO:1.

In some embodiments, the immunogenic peptide is selected from the group consisting of SEQ ID NOs: 2-13.

In some embodiments, the immunogenic carrier protein is selected from the group consisting of: CRM197; diphtheria toxin fragment B (DTFB); DTFB C8; diphtheria toxoid (DT); tetanus toxoid (TT); fragment C of TT; pertussis toxoid; cholera toxoid; E. coli LT; E. coli ST; Neisseria meningitidis outer membrane protein complex (OMPC); exotoxin A from Pseudomonas aeruginosa; mariculture keyhole limpet hemocyanin (mcKLH); and bacteriophage AP205 coat protein.

In some embodiments, the immunogenic carrier protein is conjugated at the N-terminus or C-terminus of the immunogenic peptide. In certain embodiments, the immunogenic carrier protein is conjugated at the C-terminus of the immunogenic peptide.

In some embodiments, the immunogenic carrier protein is conjugated to the immunogenic peptide with a linker. In some embodiments, the linker is selected from the group consisting of: N-γ-maleimidobutyryl-oxysuccinimide ester (GMBS); polyethylene glycol (PEG); aminohexanoic acid (Ahx); a sulfydryl-reactive crosslinker; a maleimide (MA) linker; an oligopeptide; a dendrimer; cyclodextrine; and a glycine-rich peptide.

In some embodiments, the pharmaceutical composition further comprises a spacer comprising 1-10 amino acids adjacent to the linker.

In certain embodiments, the immunogenic peptide comprises SEQ ID NO:2 and the immunogenic carrier protein is CRM197.

In other certain embodiments, the immunogenic peptide is linked to CRM197 through a GMBS linker, and the composition further comprises a glycine-cysteine spacer between the immunogenic peptide and the GMBS linker.

In some embodiments, the immunogenic peptide is monomeric. In some embodiments, the immunogenic peptide is multimeric.

In some embodiments, the pharmaceutical composition further comprises a pharmaceutically acceptable adjuvant. In some embodiments, the pharmaceutically acceptable adjuvant is selected from the group consisting of: glucopyranosol lipid adjuvant (GLA); AVT1; AVT2; AVT3, AVT4; AVT5; AVT6; AVT7; QS-21; an aluminum-based adjuvant; a saponin-based adjuvant; and a TLR7/8 agonist.

In some embodiments, the pharmaceutically acceptable adjuvant is AVT1, AVT5, or AVT7.

In another aspect, the present disclosure provides a method for preventing or treating a disease associated with amyloid deposits of Aβ in the brain of a patient in need thereof, comprising administering an effective dose of a pharmaceutical composition comprising an immunogenic peptide of at least six contiguous amino acids of SEQ ID NO:1 and an immunogenic carrier protein.

In some embodiments, the immunogenic peptide lacks 1-10 amino acids at the N-terminus or 1-33 amino acids at the C-terminus of SEQ ID NO:1. In other embodiments, the immunogenic peptide lacks 1-10 amino acids at the N-terminus and 1-33 amino acids at the C-terminus of SEQ ID NO:1.

In some embodiments, the immunogenic peptide comprises at least ten contiguous amino acids of SEQ ID NO:1.

In some embodiments, the immunogenic peptide comprises 1-5 modified amino acids. In certain embodiments, the immunogenic peptide comprises a pyroglutamate residue at amino acid position 3 and/or 11 of SEQ ID NO:1.

In some embodiments, the immunogenic peptide is selected from the group consisting of SEQ ID NOs: 2-13.

In some embodiments, the immunogenic carrier protein is selected from the group consisting of: CRM197; diphtheria toxin fragment B (DTFB); DTFB C8; diphtheria toxoid (DT); tetanus toxoid (TT); fragment C of TT; pertussis toxoid; cholera toxoid; E. coli LT; E. coli ST; Neisseria meningitidis outer membrane protein complex (OMPC); exotoxin A from Pseudomonas aeruginosa; mariculture keyhole limpet hemocyanin (mcKLH); and bacteriophage AP205 coat protein.

In some embodiments, the immunogenic carrier protein is conjugated at the N-terminus or C-terminus of the immunogenic peptide. In certain embodiments, the immunogenic carrier protein is conjugated at the C-terminus of the immunogenic peptide.

In some embodiments, the immunogenic carrier protein is conjugated to the immunogenic peptide with a linker. In some embodiments, the linker is selected from the group consisting of: N-γ-maleimidobutyryl-oxysuccinimide ester (GMBS); polyethylene glycol (PEG); aminohexanoic acid (Ahx); a sulfydryl-reactive crosslinker; a maleimide (MA) linker; an oligopeptide; a dendrimer; cyclodextrine; and a glycine-rich peptide.

In some embodiments, the pharmaceutical composition further comprises a spacer comprising 1-10 amino acids adjacent to the linker.

In certain embodiments, the immunogenic peptide comprises SEQ ID NO:2 and the immunogenic carrier protein is CRM197.

In other certain embodiments, the immunogenic peptide is linked to CRM197 through a GMBS linker, and the composition further comprises a glycine-cysteine spacer between the immunogenic peptide and the GMBS linker.

In some embodiments, the immunogenic peptide is monomeric. In some embodiments, the immunogenic peptide is multimeric.

In some embodiments, the pharmaceutical composition further comprises a pharmaceutically acceptable adjuvant. In some embodiments, the pharmaceutically acceptable adjuvant is selected from the group consisting of: glucopyranosol lipid adjuvant (GLA); AVT1; AVT2; AVT3, AVT4; AVT5; AVT6; AVT7; QS-21; an aluminum-based adjuvant; a saponin-based adjuvant; and a TLR7/8 agonist.

In some embodiments, the pharmaceutically acceptable adjuvant is AVT1, AVT5, or AVT7.

In another aspect, the present disclosure provides a use of the pharmaceutical composition described in any aspect or embodiment of the present disclosure, in the manufacture of a medicament for preventing or treating a disease associated with amyloid deposits of Aβ in the brain of a patient in need thereof.

In another aspect, the present disclosure provides a method of inducing an immune response to an Aβ peptide in a patient in need thereof, comprising administering to the patient an immunologically effective dose of the pharmaceutical composition described in any aspect or embodiment of the present disclosure.

In some embodiments, the disease associated with amyloid deposits of Aβ in the brain is a disease selected from the group consisting of: Alzheimer's Disease (AD); cerebral amyloid angiopathy (CAA); inflammatory CAA; and cerebral amyloidoma. In certain embodiments, the disease associated with amyloid deposits of Aβ in the brain is AD.

The summary of the technology described above is non-limiting and other features and advantages of the technology will be apparent from the following detailed description, and from the claims.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 shows the chemical structure of an Aβ_{pE3_9-G-C} immunogenic peptide conjugated to a CRM197 immunogenic carrier protein through a GMBS linker. SEQ ID NOS: 42 and 2 are disclosed, respectively, in order of appearance.

FIG. 2A-1 through FIG. 2A-6 is a multi-panel schematic diagram showing the synthesis of an Aβ_{pE3-9_G_C} tetramer. SEQ ID NOS: 32 and 43 are disclosed, respectively, in order of appearance. FIG. 2B-1 through FIG. 2B-7 is a multi-panel schematic diagram showing the synthesis of an Aβ_{pE3-9_G_C} octamer. SEQ ID NO: 35 is disclosed.

FIG. 3A is a line graph showing total anti-Aβ_{pE3-42} antibodies generated in non-human primates vaccinated with an pE3-9-CRM197 conjugate vaccine. FIG. 3B is a line graph showing total anti-Aβ_{pE3-42} antibodies generated in non-human primates vaccinated with an pE3-14-CRM197 conjugate vaccine.

FIG. 4A through FIG. 4D are data plots showing the measurement of Aβ₁₋₄₂- and Aβ_{pE3-42}-specific CD4 and CD8 T cell responses in vaccinated non-human primates. FIG. 4A shows the expression of cytokines by CD4 T cells following stimulation with Aβ₁₋₄₂ peptide. FIG. 4B shows the expression of cytokines by CD4 T cells following stimulation with Aβ_{pE3-42} peptide. FIG. 4C shows the expression of cytokines by CD8 T cells following stimulation with Aβ₁₋₄₂ peptide. FIG. 4D shows the expression of cytokines by CD8 T cells following stimulation with Aβ_{pE3-42} peptide. Dotted line indicates assay-defined limit of positivity. IL4=Interleukin 4, IL5=Interleukin 5, IFNg=Interferon Gamma, IL2=Interleukin 2, TNFa=Tumor necrosis factor alpha. N=5 individual animals per vaccine group.

FIG. 5A through FIG. 5D are data plots showing the measurement of Aβ₁₋₄₂- and Aβ_{pE3-42}-specific CD4 and CD8 T cell responses in vaccinated mice. FIG. 5A shows the expression of cytokines by CD4 T cells following stimulation with Aβ₁₋₄₂ peptide. FIG. 5B shows the expression of cytokines by CD4 T cells following stimulation with Aβ_{pE3-42} peptide. FIG. 5C shows the pression of cytokines by CD8 T cells following stimulation with Aβ₁₋₄₂ peptide. FIG. 5D shows the expression of cytokines by CD8 T cells following stimulation with Aβ_{pE3-42} peptide. Dotted line indicates assay-defined limit of positivity. IL4=Interleukin 4, IL10=Interleukin 10, IL5=Interleukin 5, IFNg=Interferon Gamma, IL2=Interleukin 2, TNFa=Tumor necrosis factor alpha. N=5 individual mice per vaccine group.

FIG. 6 is a bar graph showing the immunoreactivity of human brain cortex tissue from Alzheimer's Disease patients stained with increasing dilutions of sera of mice vaccination with pE3-9-C, pE3-9-G_C, pE3-10-C, pE3-11-C, pE3-12-C, pE3-13-C, pE3-14-C, pE3-15-C, pE3-15_PEG4_C, pE3-16-C, and pE3-17-C peptide vaccines. Amyloid plaque area (positive immunohistochemistry staining) for vaccinated serum is depicted in percent (y-axes) compared to the different vaccines with percentage of serum (x-axes). Dilutions in the range of 1:500 to 1:40,500 was converted to percentage of serum (x-axes).

FIG. 7 is a microglia phagocytosis assay bar graph showing the phagocytic activity of microglia upon addition of vaccinated serum, plotted as number of engulfed Aβ spots per cell (Y axis) for each of the treatment groups that were serially diluted (1:2) starting at 2% sera (X axis).

FIG. 8 is a plot of a Nano Temper differential scanning fluorescence experiment demonstrating the intrinsic fluorescence of pE3-CRM conjugates comprising various linkers when subjected to increasing temperatures.

FIG. 9 is a melting temperature plot of pE3-CRM conjugates comprising various linkers.

FIG. 10 is a plot of a dynamic light scattering experiment demonstrating the melting temperature of pE3-CRM197 conjugates comprising various linkers.

FIG. 11 is a plot of a dynamic light scattering diameter analysis of pE3-CRM conjugates comprising various linkers following the application of stir stress.

FIG. 12 is a plot of stir-stressed pE3-CRM conjugates comprising various linkers demonstrating stability as a function of fluorescence.

FIG. 13 shows a plot of an experiment demonstrating that pE3-9-CRM samples formulated with polysorbate 80 (PS80) exhibit decreased aggregation.

FIG. 14 shows a plot and results of a freeze-thaw experiment demonstrating that samples formulated with 10% sucrose exhibited decreased aggregation.

FIG. 15A through FIG. 15B are data plots of a microglia phagocytosis assay to assess functional activity of non-human primate (NHP) vaccine sera. The plots show the number of engulfed Aβ_{pE3-42} spots per cells (Y axis) plotted for each of the treatment groups over time in hours (X axis).

FIG. 16 is a data plot of a microglia phagocytosis assay in HMC3 cells to assess functional activity of NHP vaccine sera at day 42.

### DETAILED DESCRIPTION

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or amino acid residues).

All ranges disclosed herein are inclusive of the recited endpoint and independently combinable (for example, the range of “from 50 mg to 500 mg” or “50-500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” may, “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

The present disclosure is directed to compositions and methods for the treatment or prevention of diseases associated with amyloid deposits of Aβ in the brain of a patient in need thereof, such as Alzheimer's Disease. Such methods entail administering a pharmaceutical composition comprising an immunogenic fragment (i.e., an “immunogenic peptide”) of the Aβ₁₋₄₂ peptide capable of inducing a beneficial immune response in the form of antibodies to Ap. The amino acid sequence of the Aβ₁₋₄₂ peptide is set forth below.

AB₁₋₄₂:
(SEQ ID NO: 1)
DAEFRHDSGYEVHHQKLVFFAEDVGSNKGAIIGLMVGGVVIA

In one aspect, the present disclosure provides a pharmaceutical composition comprising an immunogenic peptide comprising contiguous amino acids of SEQ ID NO:1, e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 contiguous amino acids. In some embodiments, the immunogenic peptide comprises at least six contiguous amino acids of SEQ ID NO:1. In other embodiments, the immunogenic peptide comprises at least ten contiguous amino acids of SEQ ID NO:1. In other embodiments, the immunogenic peptide is selected from the group consisting of SEQ ID NOs: 2-13. In certain embodiments, the immunogenic peptide is SEQ ID NO:2.

### Immunogenic Peptides

In some embodiments, an immunogenic peptide includes gene products, naturally occurring peptides, synthetic peptides, homologs, orthologs, paralogs, fragments and other equivalents, variants, and analogs of the foregoing. A peptide may be a single molecule (i.e., monomeric) or may be a multi-molecular complex (i.e., multimeric) such as a dimer, trimer or tetramer. In some embodiments, the immunogenic peptide is monomeric. In other embodiments, the immunogenic peptide is multimeric.

Peptides may also comprise single chain peptides or multichain peptides, and may be associated or linked to each other. Most commonly, disulfide linkages are found in multichain peptides. The term “peptide” may also apply to amino acid polymers in which at least one amino acid residue is an artificial chemical analogue of a corresponding naturally-occurring amino acid.

A “peptide variant” is a molecule that differs in its amino acid sequence relative to a native sequence or a reference sequence. Amino acid sequence variants may possess substitutions, deletions, insertions, or a combination of any two or three of the foregoing, at certain positions within the amino acid sequence, as compared to a native sequence or a reference sequence. Ordinarily, variants possess at least 50% identity to a native sequence or a reference sequence. In some embodiments, variants share at least 80% identity or at least 90% identity with a native sequence or a reference sequence.

“Analogs” is meant to include peptide variants that differ by one or more amino acid alterations, for example, substitutions, additions, or deletions of amino acid residues that still maintain one or more of the properties of the parent or starting peptide.

The present disclosure provides several types of compositions that are peptide-based, including variants and derivatives. These include, for example, substitutional, insertional, deletion, and covalent variants and derivatives. The term “derivative” is synonymous with the term “variant” and generally refers to a molecule that has been “modified” and/or changed in any way relative to a reference molecule or a starting molecule. In some embodiments, the immunogenic peptide comprises 1-5 modified amino acids. In certain embodiments, the immunogenic peptide comprises a pyroglutamate residue at amino acid position 3 and/or 11 of SEQ ID NO:1.

Peptides containing substitutions, insertions and/or additions, deletions, and covalent modifications with respect to reference sequences, in particular the peptide sequences disclosed herein, are included within the scope of this disclosure. For example, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence. In some embodiments, the immunogenic peptide lacks 1-10 amino acids at the N-terminus or 1-33 amino acids at the C-terminus of SEQ ID NO:1. In other embodiments, the immunogenic peptide lacks 1-10 amino acids at the N-terminus and 1-33 amino acids at the C-terminus of SEQ ID NO:1.

“Substitutional variants” when referring to peptides are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

As used herein when referring to peptides the term “domain” refers to a motif of a peptide having one or more identifiable structural or functional characteristics or properties (e.g., binding capacity, serving as a site for protein-protein interactions)

As recognized by those skilled in the art, protein fragments, functional protein domains, and homologous proteins are also considered to be within the scope of peptides of interest. For example, provided herein is any protein fragment (meaning a peptide sequence at least one amino acid residue shorter than a reference peptide sequence but otherwise identical) of a reference protein. In another example, any protein that includes a stretch of two or more (in specific embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42) contiguous amino acids that are 40%, 50%, 60%, 70%, 80%, 90%, 95%, 96%, 97%, 98%, 99% or 100% identical to any of the sequences described herein can be utilized in accordance with the disclosure. In some embodiments, a peptide includes 1, 2, 3, 4, 5, 6, 7, 8, 9, 10, or more mutations as shown in any of the sequences provided herein or referenced herein.

Peptide present disclosure may share a certain degree of sequence similarity or identity with the reference molecules (e.g., reference peptides), for example, with art-described molecules (e.g., engineered or designed molecules or wild-type molecules). The term “identity,” as known in the art, refers to a relationship between the sequences of two or more peptides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to peptide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular peptide have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference peptide as determined by sequence alignment programs and parameters described herein and known to those skilled in the art. Such tools for alignment include those of the BLAST suite (Stephen F. Altschul, et al. (1997).” Gapped BLAST and PSI-BLAST: a new generation of protein database search programs,” Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) was developed that purportedly produces global alignment of protein sequences faster than other optimal global alignment methods, including the Needleman-Wunsch algorithm. Other tools are described herein, specifically in the definition of “identity” below.

As used herein, the term “homology” refers to the overall relatedness between polymeric molecules, e.g. between peptide molecules. Polymeric molecules (e.g. peptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, 96%, 97%, 98% or 99% identical or similar. The term “homologous” necessarily refers to a comparison between at least two sequences (peptide sequences).

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

The term “identity” refers to the overall relatedness between polymeric molecules, for example, between peptide molecules. Calculation of the percent identity of two peptide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12, 387 (1984)), BLASTP, BLASTN, and FASTA Altschul, S. F. et al., J. Molec. Biol., 215, 403 (1990)).

As used herein when referring to peptides the terms “site” as it pertains to amino acid based embodiments is used synonymously with “residue” and “amino acid side chain.” A site represents a position within a peptide or peptide that may be modified, manipulated, altered, derivatized or varied within peptide-based molecules.

As used herein the terms “termini” or “terminus” when referring to peptides refers to an extremity of a peptide. Such extremity is not limited only to the first or final site of the peptide but may include additional amino acids in the terminal regions. Peptide-based molecules may be characterized as having both an N-terminus (terminated by an amino acid with a free amino group (NH₂)) and a C-terminus (terminated by an amino acid with a free carboxyl group (COOH)). Proteins are in some cases made up of multiple peptide chains brought together by disulfide bonds or by non-covalent forces (multimers, oligomers). These proteins have multiple N- and C-termini.

Alternatively, the termini of the peptides may be modified such that they begin or end, as the case may be, with a non-peptide based moiety such as a carrier protein. Prior to conjugation with a carrier protein, the immunogenic peptide can be chemically activated using any art-known activation or coupling chemistry to make the peptide capable of reacting with the carrier protein to form a conjugate molecule. As used herein, the term “activated peptide” refers to a peptide that has been chemically modified to enable conjugation to a linker or an immunogenic carrier protein.

### Immunogenic Carrier Proteins

The immunogenic peptides disclosed herein can be conjugated to a carrier protein to improve immunogenicity in human subjects. In some embodiments of the present invention, CRM197 is used as a carrier protein. CRM197 is a non-toxic variant of diphtheria toxin (DT). The CRM197 carrier protein is a mutant form of DT that is rendered non-toxic by a single amino acid substitution in Fragment A at residue 52. In one embodiment, the CRM197 carrier protein is isolated from cultures of Corynebacterium diphtheria strain C7 (P197) grown in casamino acids and yeast extract-based medium. In another embodiment, CRM197 is prepared recombinantly in accordance with the methods described in U.S. Pat. No. 5,614,382. Typically, CRM197 is purified through a combination of ultra-filtration, ammonium sulfate precipitation, and ion-exchange chromatography. In some embodiments, CRM197 is prepared in Pseudomonas fluorescens using Pfenex Expression Technology™ (Pfenex Inc., San Diego, CA).

Other suitable carrier proteins include additional inactivated bacterial toxins such as DT, Diphtheria toxoid fragment B (DTFB), TT (tetanus toxoid) or fragment C of TT, pertussis toxoid, cholera toxoid (e.g., as described in International Patent Application Publication No. WO 2004/083251), E. coli LT (heat-labile enterotoxin), E. coli ST (heat-stable enterotoxin), and exotoxin A from Pseudomonas aeruginosa. Bacterial outer membrane proteins such as outer membrane complex c (OMPC), porins, transferrin binding proteins, pneumococcal surface protein A (PspA; See International Application Patent Publication No. WO 02/091998), pneumococcal adhesin protein (PsaA), C5a peptidase from Group A or Group B streptococcus, or Haemophilus influenzae protein D, pneumococcal pneumolysin (Kuo et al., 1995, Infect Immun 63; 2706-13) including ply detoxified in some fashion for example dPLY-GMBS (See International Patent Application Publication No. WO 04/081515) or dPLY-formol, PhtX, including PhtA, PhtB, PhtD, PhtE and fusions of Pht proteins for example PhtDE fusions, PhtBE fusions (See International Patent Application Publication Nos. WO 01/98334 and WO 03/54007), can also be used. Other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D; See, e.g., European Patent No. EP 0 594 610 B), or immunologically functional equivalents thereof, synthetic peptides (See European Patent Nos. EP0378881 and EP0427347), heat shock proteins (See International Patent Application Publication Nos. WO 93/17712 and WO 94/03208), pertussis proteins (See International Patent Application Publication No. WO 98/58668 and European Patent No. EP0471177), cytokines, lymphokines, growth factors or hormones (See International Patent Application Publication No. WO 91/01146), artificial proteins comprising multiple human CD4+ T cell epitopes from various pathogen derived antigens (See Falugi et al., 2001, Eur J Immunol 31:3816-3824) such as N19 protein (See Baraldoi et al., 2004, Infect Immun 72:4884-7), iron uptake proteins (See International Patent Application Publication No. WO 01/72337), toxin A or B of C. difficile (See International Patent Publication No. WO 00/61761), and flagellin (See Ben-Yedidia et al., 1998, Immunol Lett 64:9) can also be used as carrier proteins.

Other DT mutants can also be used as the carrier protein, such as CRM176, CRM228, CRM45 (Uchida et al., 1973, J Biol Chem 218:3838-3844); CRM9, CRM45, CRM102, CRM103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or U.S. Pat. No. 4,950,740; mutation of at least one or more residues Lys 516, Lys 526, Phe 530 and/or Lys 534 and other mutations disclosed in U.S. Pat. No. 5,917,017 or U.S. Pat. No. 6,455,673; or fragment disclosed in U.S. Pat. No. 5,843,711.

In some embodiments, the immunogenic carrier protein is selected from the group consisting of: CRM197; diphtheria toxin fragment B (DTFB); DTFB C8; diphtheria toxoid (DT); tetanus toxoid (TT); fragment C of TT; pertussis toxoid; cholera toxoid; E. coli LT; E. coli ST; Neisseria meningitidis outer membrane protein complex (OMPC); exotoxin A from Pseudomonas aeruginosa; mariculture keyhole limpet hemocyanin (mcKLH); and virus-like particles derived from the bacteriophage AP205 coat protein.

In certain embodiments, the immunogenic peptide comprises SEQ ID NO:2 and the immunogenic carrier protein is CRM197. In some embodiments, the immunogenic carrier protein is conjugated at the N-terminus or C-terminus of the immunogenic peptide. In certain embodiments, the immunogenic carrier protein is conjugated at the C-terminus of the immunogenic peptide.

In some embodiments, the immunogenic carrier protein is conjugated to the immunogenic peptide with a linker. In some embodiments, the linker is selected from the group consisting of: N-γ-maleimidobutyryl-oxysuccinimide ester (GMBS); polyethylene glycol (PEG); aminohexanoic acid (Ahx); a sulfydryl-reactive crosslinker; a maleimide (MA) linker; an oligopeptide; a dendrimer; cyclodextrine; and a glycine-rich peptide. In some embodiments, the pharmaceutical compositions of the present disclosure further comprise a spacer comprising 1-10 amino acids adjacent to the linker. In certain embodiments, the immunogenic peptide is linked to CRM197 through a GMBS linker, and the composition further comprises a glycine-cysteine spacer between the immunogenic peptide and the GMBS linker.

Following the conjugation, the peptide-conjugates may be purified from unconjugated material by a variety of techniques known to the skilled person. These techniques include dialysis, concentration/diafiltration operations, tangential flow filtration, ultrafiltration, precipitation/elution, column chromatography (ion exchange chromatography, multimodal ion exchange chromatography, DEAE, or hydrophobic interaction chromatography), and depth filtration. See, e.g., U.S. Pat. No. 6,146,902. In an embodiment, the glycoconjugates are purified by diafiltration or ion exchange chromatography or size exclusion chromatography.

### Adjuvants

In some embodiments, the pharmaceutical composition further comprises a pharmaceutically acceptable adjuvant. As defined herein, an “adjuvant” is a substance that serves to enhance the immunogenicity of an immunogenic composition of the invention. An immune adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan.

Pharmaceutically acceptable adjuvants to enhance effectiveness of the disclosed immunogenic peptide compositions include, but are not limited to, those described in Example 7 and below.

Aluminum salts (alum), such as aluminum hydroxide, aluminum phosphate, aluminum sulfate, etc. Aluminum salt adjuvant may be an alum-precipitated vaccine or an alum-adsorbed vaccine. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493). The aluminum salt includes, but is not limited to, hydrated alumina, alumina hydrate, alumina trihydrate (ATH), aluminum hydrate, aluminum trihydrate, Alhydrogel®, Superfos, Amphogel®, aluminum (III) hydroxide, aluminum hydroxyphosphate (Aluminum Phosphate Adjuvant (APA)), amorphous alumina, trihydrated alumina, or trihydroxyaluminum.

APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a monodisperse particle size distribution. The product is then diafiltered against physiological saline and steam sterilized.

In some embodiments, a commercially available Al(OH)₃ (e.g. Alhydrogel® or Superfos of Denmark/Accurate Chemical and Scientific Co., Westbury, NY) is used to adsorb proteins. Adsorption of protein is dependent, in another embodiment, on the pI (Isoelectric pH) of the protein and the pH of the medium. A protein with a lower pI adsorbs to the positively charged aluminum ion more strongly than a protein with a higher pI. Aluminum salts may establish a depot of antigen that is released slowly over a period of 2-3 weeks, be involved in nonspecific activation of macrophages and complement activation, and/or stimulate innate immune mechanism (possibly through stimulation of uric acid). See, e.g., Lambrecht et al., 2009, Curr Opin Immunol 21:23.

Oil-in-water emulsion formulations (with or without other specific immunostimulating agents such as muramyl peptides or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, MA), (b) SAF, containing 10% Squalene, 0.4% Tween 80, 5% pluronic-blocked polymer L121, and thr-MDP either microfluidized into a submicron emulsion or vortexed to generate a larger particle size emulsion, (c) Ribi™ adjuvant system (RAS), (Corixa, Hamilton, MT) containing 2% Squalene, 0.2% Tween 80, and one or more bacterial cell wall components from the group consisting of 3-O-deacylated monophosphorylipid A (MPL™) described in U.S. Pat. No. 4,912,094, trehalose dimycolate (TDM), and cell wall skeleton (CWS), preferably MPL+CWS (Detox™); and (d) a Montanide ISA. Muramyl peptides include, but are not limited to, N-acetyl-muramyl-L-threonyl-D-isoglutamine (thr-MDP), N-acetyl-normuramyl-L-alanine-2-(1′,2′-dipalmitoyl-sn-glycero-3-hydroxyphosphoryloxy)-ethylamine (MTP-PE), etc. In certain embodiments, AVT7 is an exemplary oil-in-water emulsion adjuvant comprising Squalene, SPAN-85, and PS-20 components stabilized in an aqueous buffer at pH 5.8.

Saponin adjuvants, such as Quil A or STIMULON™ QS-21 (Antigenics, Framingham, MA) (see, e.g., U.S. Pat. No. 5,057,540) may be used or particles generated therefrom such as ISCOM (immunostimulating complexes formed by the combination of cholesterol, saponin, phospholipid, and amphipathic proteins) and Iscomatrix® (having essentially the same structure as an ISCOM but without the protein).

Bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-tetradecanoyloxytetradecanoylamino]ethyl 2-Deoxy-4-O-phosphono-3-O-[(R)-3-tetradecanoyloxytetradecanoyl]-2-[(R)-3-tetradecanoyloxytetradecanoylamino]-b-D-glucopyranoside, which is also known as 529 (formerly known as RC529), which is formulated as an aqueous form or as a stable emulsion.

Synthetic polynucleotides such as oligonucleotides containing CpG motif(s) (U.S. Pat. No. 6,207,646).

Stable nanoemulsion or SNE refers to a formulation of emulsifiers and/or solubilizers and/or surfactants and/or lipids that have adjuvant properties in a conjugate vaccine. In exemplary embodiments, SNE refers to a SNE adjuvant formulation comprising 1) sorbitan trioleate (SPAN-85); 2) polysorbate-20 (PS-20); 3) Squalene; and an optional 4) cationic lipid, as described in WO2022169789A1, the contents of which is herein incorporated in its entirety. In certain embodiments, the SNE comprises 6 g/mL-14 mg/mL SPAN-85, 6 g/mL-14 mg/mL PS-20 or PS-80 and 60 g/mL-34 mg/mL of Squalene. In some embodiments, the SNE further comprises 30 g/mL-2.4 mg/mL of cationic lipid. In some embodiments, the cationic lipid is (13Z,16Z)-N,N-dimethyl-3-nonyldocosa-13,16-dien-1-amine.

Cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc.

A CpG-containing nucleotide sequence, for example, a CpG-containing oligonucleotide, in particular, a CpG-containing oligodeoxynucleotide (CpG ODN). In another embodiment, the adjuvant is ODN 1826, which may be acquired from Coley Pharmaceutical Group. “CpG-containing nucleotide,” “CpG-containing oligonucleotide,” “CpG oligonucleotide,” and similar terms refer to a nucleotide molecule of 6-50 nucleotides in length that contains an unmethylated CpG moiety. See, e.g., Wang et al., 2003, Vaccine 21:4297. In another embodiment, any other art-accepted definition of the terms is intended. CpG-containing oligonucleotides include modified oligonucleotides using any synthetic internucleoside linkages, modified base and/or modified sugar. Methods for use of CpG oligonucleotides are well known in the art and are described, for example, in Sur et al., 1999, J Immunol. 162:6284-93; Verthelyi, 2006, Methods Mol Med. 127:139-58; and Yasuda et al., 2006, Crit Rev Ther Drug Carrier Syst. 23:89-110

Complement, such as a trimer of complement component C3d.

Liquid nanoparticle (LNP) adjuvants are a class of adjuvants containing cationic lipid/cholesterol/DSPC/PEG-DMG, as described in WO2015/130584A2, the contents of which is herein incorporated in its entirety. Exemplary LNP adjuvants may include cationic lipid/cholesterol/DSPC/PEG-DMG at about the following molar ratios: 59/30/10/1; 58/30/10/2; 43/41/15/1; 42/41/15/2; 40/48/10/2; 39/41/19/1; 38/41/19/2; 34/41/24/1; and 33/41/24/2. In certain embodiments, AVT1 is an exemplary LNP adjuvant comprising lipid, cholesterol, DSPC, and PEG-DMG components.

In an embodiment, the adjuvant is a mixture of two, three, or more of the above adjuvants, e.g., SBAS2 (an oil-in-water emulsion also containing 3-deacylated monophosphoryl lipid A and QS21).

### Methods of Prevention and Treatment

Provided herein are compositions (e.g., pharmaceutical compositions, including immunogenic peptides, conjugates, and vaccines), methods, kits, and reagents for the prevention, treatment, or diagnosis of neurodegenerative diseases associated with amyloid deposits of Aβ in the brain of a patient, such as Alzheimer's Disease, in humans. In some embodiments, the methods of the disclosure comprise administering an effective dose of a pharmaceutical composition, e.g., a vaccine, comprising an immunogenic peptide of at least six contiguous amino acids of SEQ ID NO:1 and an immunogenic carrier protein, to a patient in need thereof.

In some embodiments, the immunogenic peptide lacks 1-10 amino acids at the N-terminus or 1-33 amino acids at the C-terminus of SEQ ID NO:1. In other embodiments, the immunogenic peptide lacks 1-10 amino acids at the N-terminus and 1-33 amino acids at the C-terminus of SEQ ID NO:1.

In some embodiments, the immunogenic peptide comprises at least ten contiguous amino acids of SEQ ID NO:1.

In some embodiments, the immunogenic peptide comprises 1-5 modified amino acids. In certain embodiments, the immunogenic peptide comprises a pyroglutamate residue at amino acid position 3 and/or 11 of SEQ ID NO:1.

In some embodiments, the immunogenic peptide is selected from the group consisting of SEQ ID NOs: 2-13.

In some embodiments, the immunogenic carrier protein is selected from the group consisting of: CRM197; diphtheria toxin fragment B (DTFB); DTFB C8; diphtheria toxoid (DT); tetanus toxoid (TT); fragment C of TT; pertussis toxoid; cholera toxoid; E. coli LT; E. coli ST; Neisseria meningitidis outer membrane protein complex (OMPC); exotoxin A from Pseudomonas aeruginosa; mariculture keyhole limpet hemocyanin (mcKLH); and bacteriophage AP205 coat protein.

In some embodiments, the immunogenic carrier protein is conjugated at the N-terminus or C-terminus of the immunogenic peptide. In certain embodiments, the immunogenic carrier protein is conjugated at the C-terminus of the immunogenic peptide.

In some embodiments, the immunogenic carrier protein is conjugated to the immunogenic peptide with a linker. In some embodiments, the linker is selected from the group consisting of: N-γ-maleimidobutyryl-oxysuccinimide ester (GMBS); polyethylene glycol (PEG); aminohexanoic acid (Ahx); a sulfydryl-reactive crosslinker; a maleimide (MA) linker; an oligopeptide; a dendrimer; cyclodextrine; and a glycine-rich peptide.

In some embodiments, the pharmaceutical composition further comprises a spacer comprising 1-10 amino acids adjacent to the linker.

In certain embodiments, the immunogenic peptide comprises SEQ ID NO:2 and the immunogenic carrier protein is CRM197.

In other certain embodiments, the immunogenic peptide is linked to CRM197 through a GMBS linker, and the composition further comprises a glycine-cysteine spacer between the immunogenic peptide and the GMBS linker.

In some embodiments, the immunogenic peptide is monomeric. In some embodiments, the immunogenic peptide is multimeric.

As used herein, the term “treat” or “treating” means to administer a therapeutic moiety, such as a composition containing any of the immunogenic peptides, conjugates, or vaccines of the present invention, internally or externally to a subject or patient having one or more disease symptoms, or being diagnosed as having a disease, for which the therapeutic moiety has therapeutic activity. In specific embodiments, the immunogenic peptides, conjugates, or vaccines can be administered topically, subcutaneously, intramuscular, intradermally, intravenously, or systemically. Typically, the moiety is administered in an amount effective to: (i) alleviate one or more disease symptoms in the treated subject or population, whether by inducing the regression of or inhibiting the progression of such symptom(s) by any clinically measurable degree, or (ii) inhibiting or reducing the severity of the disease in an individual. The amount of a therapeutic moiety that is effective to alleviate any particular disease symptom, and/or to inhibit or reduce the severity of the disease, including in specific embodiments neurodegenerative disease, in the individual may vary according to factors such as the injury or disease state, age, and/or weight of the individual, and the ability of the therapeutic moiety to elicit a desired response in the individual. Whether one or more disease symptoms have been alleviated or the severity of the disease inhibited or reduced can be assessed by any clinical measurement typically used by physicians or other skilled healthcare providers to assess the severity or progression status of the symptom(s) or disease. Treatment with the immunogenic peptides, conjugates, or vaccines could also be combined with other interventions (in specific embodiments, antibodies, nucleic acids, additional vaccines, and small molecule compounds) to treat other symptoms or diseases.

As used herein, the term “subject, individual,” or “patient in need thereof” means a human or animal subject, individual, or patient exhibiting a symptom of, or diagnosed as having a disease associated with amyloid deposits of Aβ in the brain of a patient, such as Alzheimer's Disease, that will be the subject of the treatment.

### Vaccines

Vaccines of the present disclosure may be administered prophylactically or therapeutically as part of an active immunization scheme to healthy individuals or early after diagnosis of a neurodegenerative disease. In some embodiments, the amount of vaccine of the present disclosure provided to a cell, a tissue, or a subject may be an amount effective for immune prophylaxis or treatment.

Vaccines may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years, or more than 99 years. In some embodiments, the time of administration between the initial administration of the prophylactic composition and the booster may be, but is not limited to, 1 week, 2 weeks, 3 weeks, 1 month, 2 months, 3 months, 4 months, 5 months, 6 months, or 1 year.

In some embodiments, vaccines may be administered orally, intramuscularly, intradermally, or intranasally, similarly to the administration of inactivated vaccines known in the art. In some embodiments, vaccines are administered intramuscularly.

Vaccines may be utilized in various settings depending on the prevalence of the underlying disease, family history, or degree or level of unmet medical need. As a non-limiting example, the vaccines may be utilized to treat and/or prevent a class of neurodegenerative diseases associated with amyloid deposits of Aβ in the brain of a patient. Vaccines have superior properties in that they produce much larger antibody titers and produce responses early compared to commercially available non-immunogenic compositions.

Provided herein are pharmaceutical compositions including vaccines optionally in combination with one or more pharmaceutically acceptable excipients.

Vaccines may be formulated or administered alone or in conjunction with one or more other components. For instance, vaccines (vaccine compositions) may comprise other components including, but not limited to, pharmaceutically acceptable adjuvants. In some embodiments, the pharmaceutically acceptable adjuvant is selected from the group consisting of: glucopyranosol lipid adjuvant (GLA); AVT1; AVT2; AVT3, AVT4; AVT5; AVT6; AVT7; QS-21; an aluminum-based adjuvant; a saponin-based adjuvant; and a TLR7/8 agonist.

In some embodiments, the vaccines do not include an adjuvant (they are adjuvant free).

Vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free, or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety). In some embodiments, vaccines are administered to humans, such as human patients or subjects.

Formulations of the vaccine compositions described herein may be prepared by any method known or hereafter developed in the art of pharmacology. In general, such preparatory methods include the step of bringing the active ingredient (e.g., the immunogenic peptide conjugate) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, or at least 80% (w/w) active ingredient.

### Modes of Vaccine Administration

Vaccines may be administered by any route which results in a therapeutically effective outcome. These include, but are not limited to, oral, intradermal, intramuscular, intranasal, and/or subcutaneous administration. The present disclosure provides methods comprising administering vaccines to a subject in need thereof. The exact amount required will vary from subject to subject, depending on the species, age, and general condition of the subject, the severity of the disease, the particular composition, its mode of administration, its mode of activity, and the like. Vaccine compositions are typically formulated in dosage unit form for ease of administration and uniformity of dosage. It will be understood, however, that the total daily usage of vaccine compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific vaccine employed; and like factors well known in the medical arts.

In some embodiments, vaccine compositions may be administered at dosage levels sufficient to obtain the desired therapeutic, diagnostic, prophylactic, or imaging effect (See, e.g., the range of unit doses described in International Publication No WO2013078199, the contents of which are herein incorporated by reference in their entirety). The desired dosage may be delivered three times a day, two times a day, once a day, every other day, every third day, every fourth day, every fifth day, every week, every two weeks, every three weeks, every four weeks, every two months, every three months, every four months, every five months, every six months, etc. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

A vaccine pharmaceutical composition described herein can be formulated into a dosage form described herein, such as an oral, intranasal, intratracheal, or injectable (e.g., intravenous, intraocular, intravitreal, intramuscular, intradermal, intracardiac, intraperitoneal, intranasal and subcutaneous).

### Vaccine Formulations and Methods of Use

Some aspects of the present disclosure provide vaccine formulations, wherein the vaccine is formulated in an effective amount to produce an antigen specific immune response in a subject (e.g., production of antibodies specific to an Aβ antigenic peptide). “An effective amount” is a dose of a vaccine effective to produce an antigen-specific immune response. Also provided herein are methods of inducing an antigen-specific immune response in a subject.

In some embodiments, the antigen-specific immune response is characterized by measuring an anti-Aβ antigenic peptide antibody titer produced in a subject administered a vaccine as provided herein. An antibody titer is a measurement of the amount of antibodies within a subject, for example, antibodies that are specific to a particular antigen (e.g., an Aβ antigenic peptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has a neurodegenerative disease or to determine whether immunizations are required. In some embodiments, an antibody titer is used to determine the strength of an autoimmune response, to determine whether a booster immunization is needed, to determine whether a previous vaccine was effective, and to identify disease onset or progression. In accordance with the present disclosure, an antibody titer may be used to determine the strength of an immune response induced in a subject by vaccine.

The summary of the technology described above is non-limiting and other features and advantages of the technology will be apparent from the following detailed description, and from the claims.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

### EXAMPLES

The following examples are meant to be illustrative and should not be construed as further limiting. The contents of the figures and all references, patents, and published patent applications cited throughout this application are expressly incorporated herein by reference.

Abbreviations used in the following examples may include, but are not limited to, those shown in Table 1 below.

TABLE 1
Abbreviations

| | |
|---|---|
| DCM | Dichloromethane |
| DIC | N,N-Diisopropylcarbodiimide |
| DIPEA | N,N-Diisopropylethylamine |
| DMF | Dimethylformamide |
| DODT | 3,6-dioxa-1,8-octanedithiol |
| DTT | Dithiothreitol |
| EDTA | Ethylenediaminetetraacetic acid |
| eq | Equivalent(s) |
| Fmoc | Fluorenylmethyloxycarbonyl |
| hr(s) | Hour(s) |
| HATU | 1-[Bis(dimethylamino)methylene]-1H-1,2,3-triazolo[4,5- |
| | b]pyridinium 3-oxide hexafluorophosphate |
| HOBt | Hydroxy benzotriazole |
| HPLC | High performance liquid chromatography |
| LCMS | Liquid chromatography-mass spectrometry |
| min | Minute(s) |
| MeCN | Acetonitrile |
| MeOH | Methanol |
| MS | Mass spectrometry |
| MWCO | Molecular weight cut-off |
| m/z | Mass to charge ratio |
| Oxyma | Ethyl cyanohydroxyiminoacetate |
| RT | Room temperature |
| sec | Second(s) |
| SPPS | Solid phase peptide synthesis |
| TIPS | Triisopropylsilane |
| TFA | Trifluoroacetic acid |
| THF | Tetrahydrofuran |
| THPTA | Tris(3-hydroxypropyltriazolylmethyl)amine |
| TLR4 | Toll-like receptor 4 |
| TLR7/8 | Toll-like receptor 7/8 |
| Rink amide | 4-(2′,4′-Dimethoxyphenyl-Fmoc-aminomethyl)- |
| MBHA | phenoxyacetamido-norleucyl-MBHA resin |
| Ahx | 6-aminohexanoic acid |
| Arg | Arginine |
| Cys | Cysteine |
| Asp | Aspartic Acid |
| Gln | Glutamine |
| Glu | Glutamic Acid |
| Gly | Glycine |
| Lys | Lysine |
| His | Histidine |
| Leu | Leucine |
| Ile | Isoluecine |
| Asn | Asparagine |
| Phe | Phenylalanine |
| Pro | Proline |
| Ser | Serine |
| Thr | Threonine |
| Trp | Tryptophan |
| Tyr | Tyrosine |
| Val | Valine |
| Ala | Alanine |
| Met | Methionine |
| pE or pyroE | Pyroglutamic acid |

### Example 1: Synthesis of AB Peptides

To assess the immunogenicity of Aβ_{pE3} peptide antigens, synthetic peptide sequences of varying lengths were prepared from the natural Aβ_{pE3} sequence. Twelve peptide sequences spanning Aβ_{pE3-9} through Aβ_{pE3-17} were synthesized and are shown in Table 2 below. Peptide sequences may also be linked together to form multimeric peptides as described in Example 2. Each peptide or multimeric peptide contained a linker and a chemically reactive group at the C-terminus that enables chemical conjugation to a carrier protein or particle. The linker and chemically reactive group may vary depending on the peptide sequence and carrier protein or particle used.

Aβ peptide synthesis was completed in three steps unless otherwise indicated. The following protected natural amino acids were used: Fmoc-Arg(Pbf)-OH; Fmoc-Asp(OMpe)-OH, Fmoc-Cys(Trt)-OH; Fmoc-Gln(Trt)-OH; Fmoc-Gly-OH; Fmoc-Glu(OtBu)-OH; Fmoc-His(Trt)-OH; Fmoc-Phe-OH; Fmoc-Ser(t-Bu)-OH; Fmoc-Tyr(t-Bu)-OH; and Fmoc-Val-OH.

Step 1: synthesis. The peptides were synthesized using Fmoc/t-Bu chemistry on Rink amide resin (0.25 mmol, 0.35-0.58 mmol/g). Amino acids were incorporated to the resin sequentially through either a single or double coupling cycle. The synthesis was performed either manually or automatically as described in Protocol A and Protocol B, respectively.

For Protocol A, Rink amide MBHA resin was loaded into a glass sintered vessel equipped with a frit. Reactions were performed at room temperature on an orbital shaker. Typical deprotection and coupling reaction conditions were as follows. For deprotection, the reaction mixture was allowed to react in 20% (v/v) piperidine for 20 minutes. The mixture was filtered, and the peptidyl resin was washed with DMF (5×15 mL), DCM (5×15 mL), and DMF (5×15 mL). For coupling, either a pre-activated DMF solution of three equivalents of amino acid, three equivalents of DIC, and three equivalents of HOBt, or a DMF solution of three equivalents of HATU, three equivalents of HOBt, and six equivalents of DIPEA was added to the resin, and the mixture was allowed to react for three hours at room temperature. After a Kaiser test indicated the completion of the reaction, the mixture was filtered and the peptidyl resin was washed with DMF (5×15 mL), DCM (5×15 mL), and DMF (5×15 mL).

For Protocol B, Rink amide MBHA resin was loaded onto a CEM's Liberty Blue microwave assisted synthesizer (CEM Corp, Matthews, NC, USA). Typical deprotection and coupling reaction conditions were as follows. For deprotection, the reaction mixture was allowed to react in 20% (v/v) piperidine plus 0.1 M HOBt in DMF (25 seconds at 75° C.; followed by 65 seconds at 90° C.), followed by 3-4 cycles of DMF wash. For each coupling cycle, four equivalents of amino acid in 5 ml of 0.2 M DMF-stock solution were delivered to the resin, followed by the addition of four equivalents of DIC in 1 ml of 1M DMF stock solution, and four equivalents of Oxyma in 1 ml of 1M DMF stock solution. All residues except for His were allowed to react for 2 minutes at 90° C., followed by 3-4 cycles of DMF wash. His was allowed to react for 10 mins at 50° C., followed by 3-4 cycles of DMF wash.

Step 2: cleavage. The above peptidyl resin was washed by DMF and DCM (3×15 ml) each, followed by MeOH (15 ml) and diethyl ether (2×15 ml). Finally, the resin was dried overnight under vacuum. Once dried, the resin was treated with 10 ml of reagent cocktail (v/v) (95% TFA, 2.5% TIPS, 2.5% DODT) at room temperature for three hours. Cleavage mixture was collected by filtration and the resin was washed with TFA. Combined filtrate containing peptide was precipitated with a sufficient amount of cold (0° C.) diethyl ether (about 9× filtrate volume). The precipitated peptide was centrifuged (4000 rpm) and the supernatant was removed. Fresh diethyl ether was added to the peptide and re-centrifuged. This process was repeated three times. The precipitated peptide was then dried under high vacuum overnight to obtain the crude product as a white solid. HPLC and LCMS analysis was used to confirm the quality and identity of the desired peptide. Total crude weight obtained was 0.35 g.

Step 3: purification. The above crude peptide was dissolved in water and MeCN and purified by a Shimazu Prominence LC-20AP equipped with SPD-M20A diode array detector (Shimadzu Corp. Japan). HPLC conditions were as follows. Column: Phenomenex Luna C18 5u 100A 250*21.2 mm; buffer A: 0.01% TFA in H2O; buffer B: 100% MeCN; flow rate: 15 ml/min; gradient: 2-25% in 30 min. The HPLC fractions containing a pure peptide product were pooled and lyophilized to give a final peptide product.

TABLE 2
Pyroglutamate-modified Aβ peptides

| SEQ ID NO | Peptide Sequence |
|---|---|
| 2 | [pE]FRHDSG |
| 3 | [pE]FRHDSGGC |
| 4 | [pE]FRHDSGY |
| 5 | [pE]FRHDSGYE |
| 6 | [pE]FRHDSGYEV |
| 7 | [pE]FRHDSGYEVH |
| 8 | [pE]FRHDSGYEVHH |
| 9 | [pE]FRHDSGYEVHHQ |
| 10 | [pE]FRHDSGYEVHHQK |
| 11 | [pE]FRHDSGYEVHHQKL |
| 12 | [pE]VHHQKL |
| 13 | [pE]VHHQKLGC |

### Example 2: Synthesis of Multimeric AB Peptides

This antigen design aimed to mimic pathogenic amyloid beta plaques to elicit target-specific immune responses. Antigenic Aβ peptides, e.g., pE3-X, can be further modified into multimers to better capture the structural features of amyloid beta plaques, potentially improving the immunogenicity and specificity of the vaccines. These multimeric preparations can be achieved by crosslinking Aβ peptide monomers through chemical synthesis, e.g., using linkers such as PEG, oligopeptides, dendrimers, cyclodextrin, etc.

THPTA (Tris(3-hydroxypropyltriazolylmethyl)amine, 18.6 mg, 42.8 M), copper sulfate (1.37 mg, 8.6 M), and a stir bar were added to a 5 mL glass vial containing 450 μL water. The reaction was stirred for 2 minutes at room temperature, followed by the addition of sodium ascorbate (42.4 mg, 214 μM) and then Ac-SH-alkyne (18.6 mg, 6 μM, prepared as solution in 250 L acetonitrile). After stirring for 5 minutes, the water-acetonitrile reaction mixture was added Azido-bis-amine (30 mg, 42. μM). The product formation, Ac-SH-bis-amine, was confirmed by LC-MS after a 1-hour reaction at room temperature with all the starting materials being consumed. The mixture was subjected to a column chromatography purification using a Combi-Flash system (C18 reverse-phase, 0-60% acetonitrile over 10 minutes, RT=8 minute). The collected product fractions were combined and lyophilized overnight. A colorless, oil-like product was obtained (yield=10%). Calculated [M+H]+=991.58; observed [M+H]+=991.26.

NHS-bis-mal (2 mg, 2 μM), Ac-SH-bis-amine (1 mg, 1 μM), and 120 μL 50 mM sodium phosphate buffer (pH 8) were added to a 0.6 mL microtube. The reaction was incubated at room temperature overnight with rotation. Next, pE3-9 peptide (4 mg, 4 μM) was added to the reaction, followed by a 4-hour incubation at room temperature with rotation. Formation of the desired product was confirmed by LC-MS. The product purification was carried out by using a 3K MWCO (molecular weight cut-off) column and centrifugation. The solvent was then removed by lyophilization to obtain the product as light-pink solid. Calculated [M+4H]⁴⁺=1677.75; observed [M+4H]⁴⁺=1677.46.

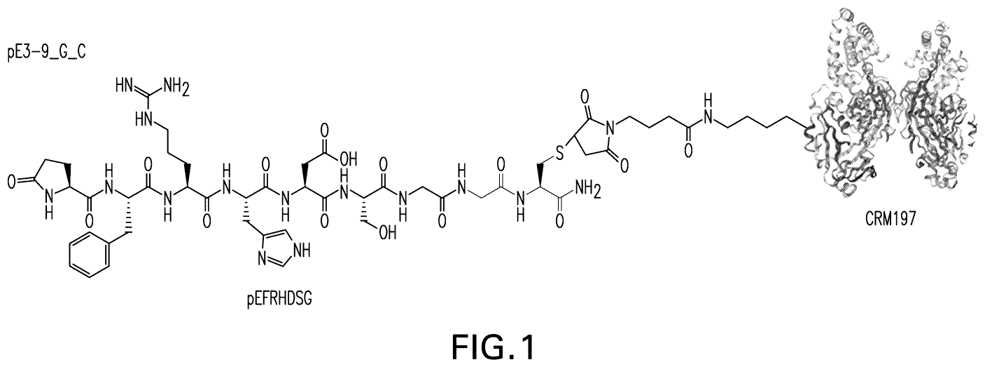

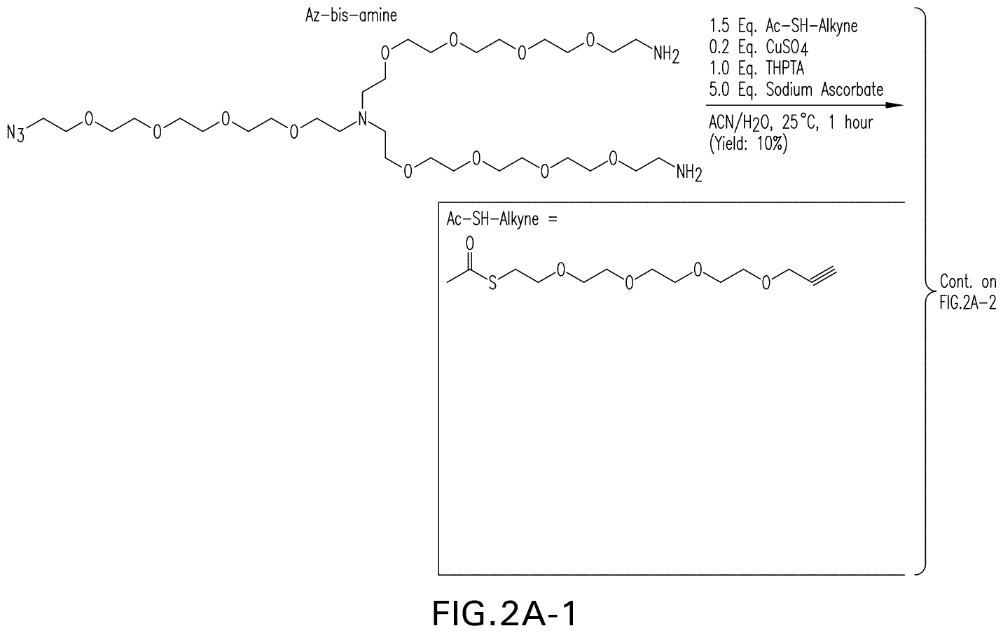

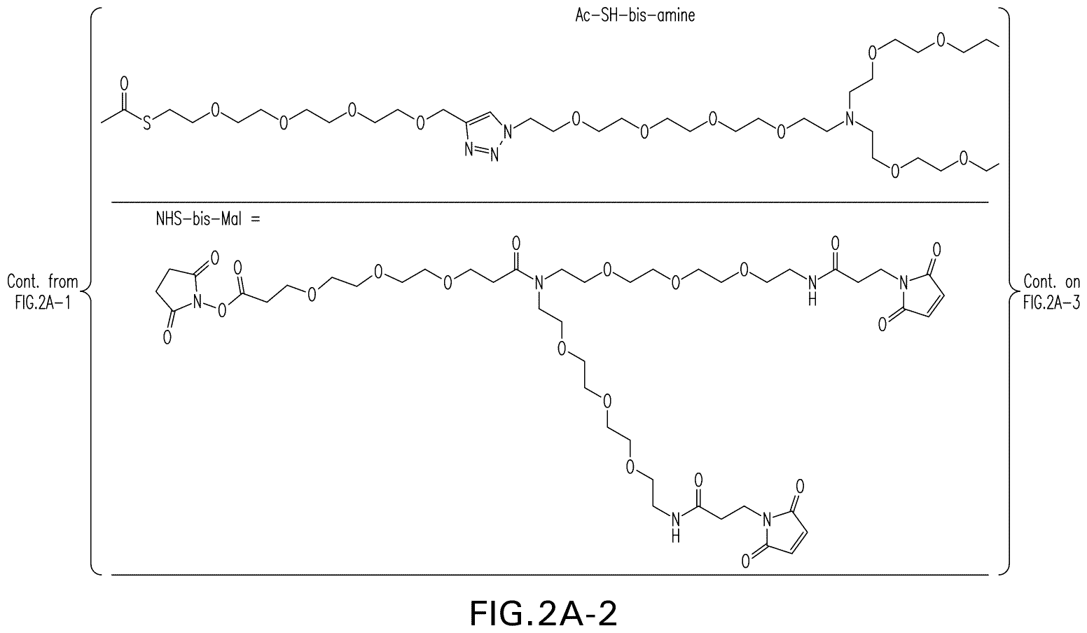

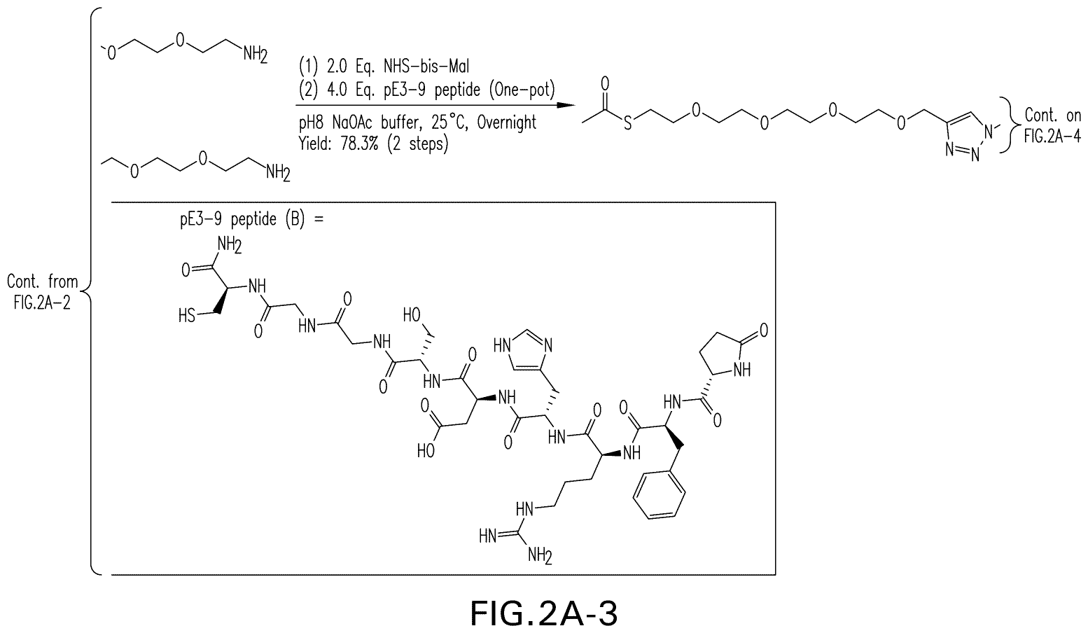

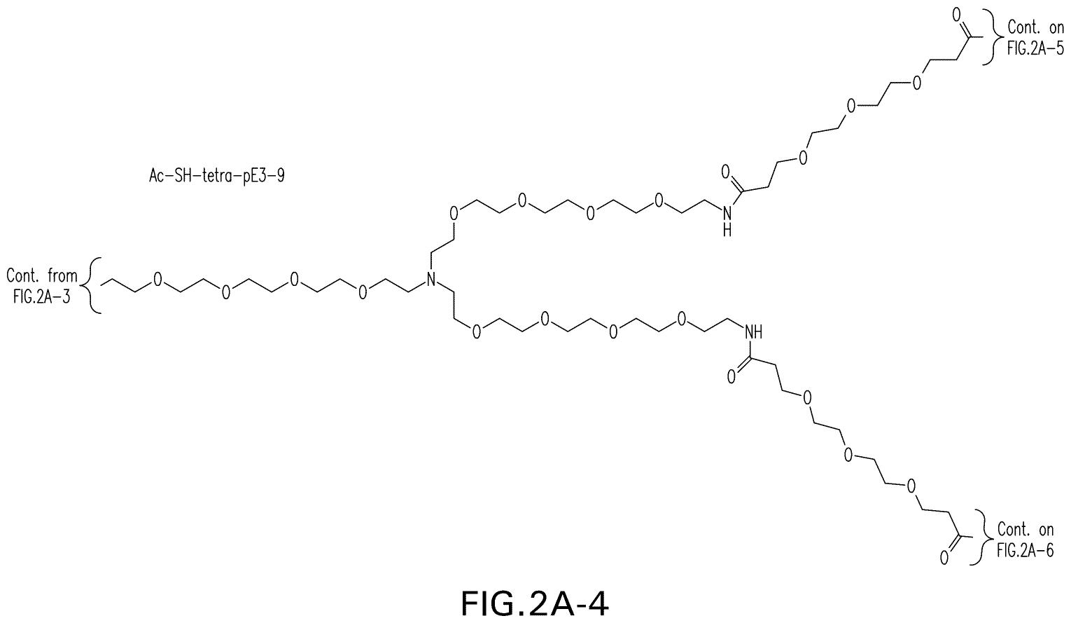

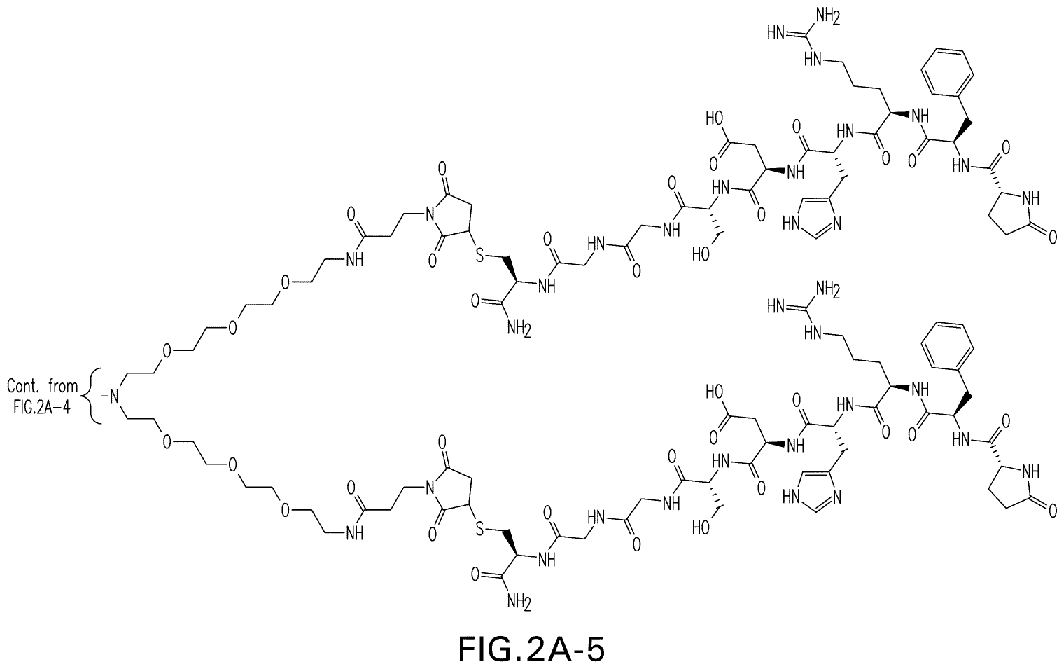

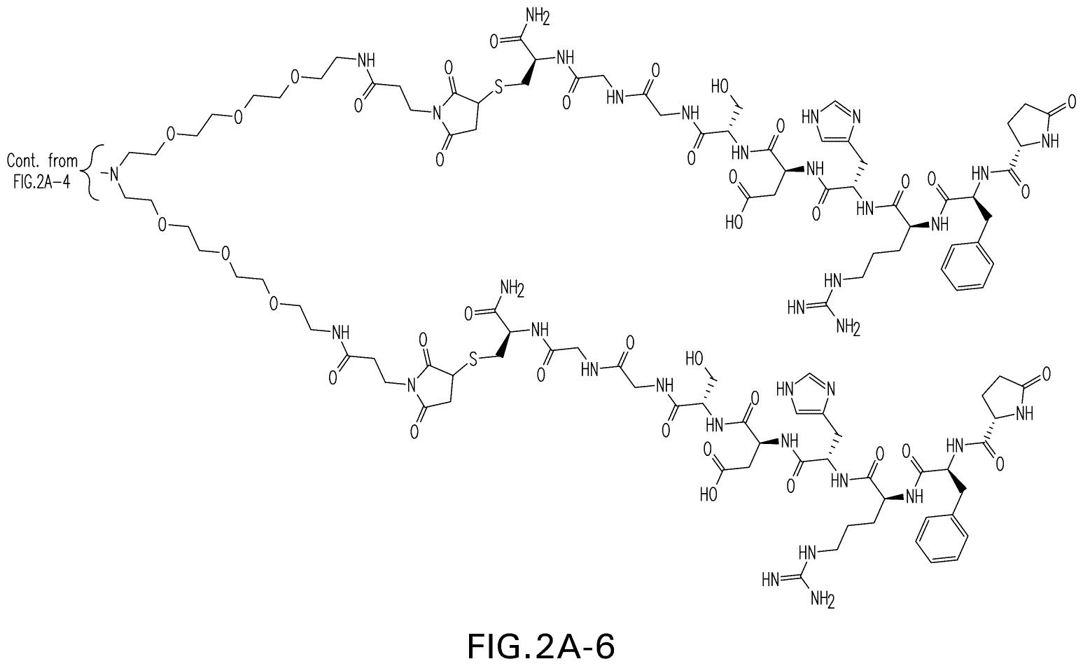

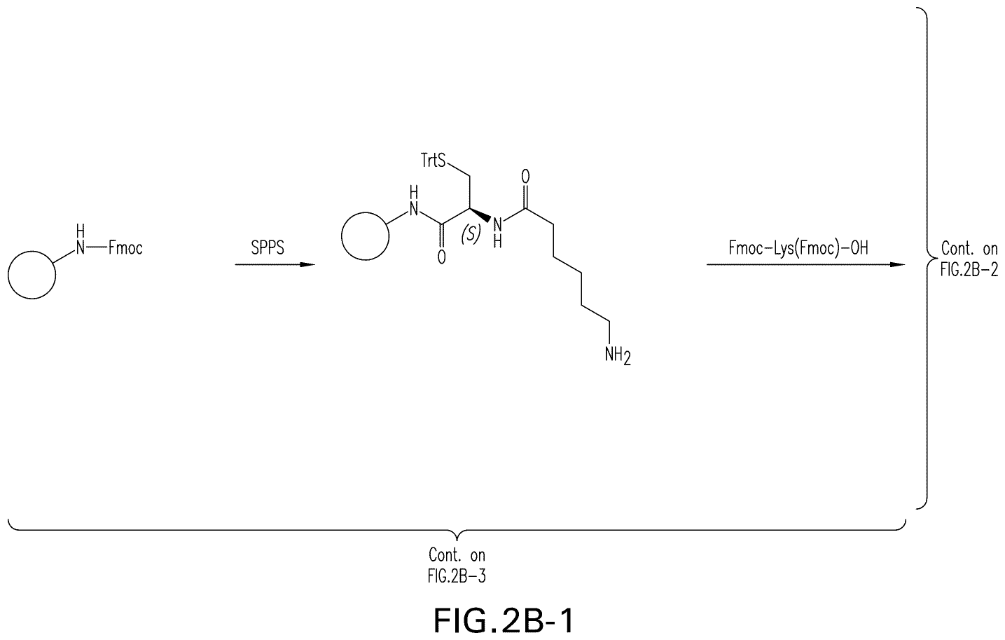

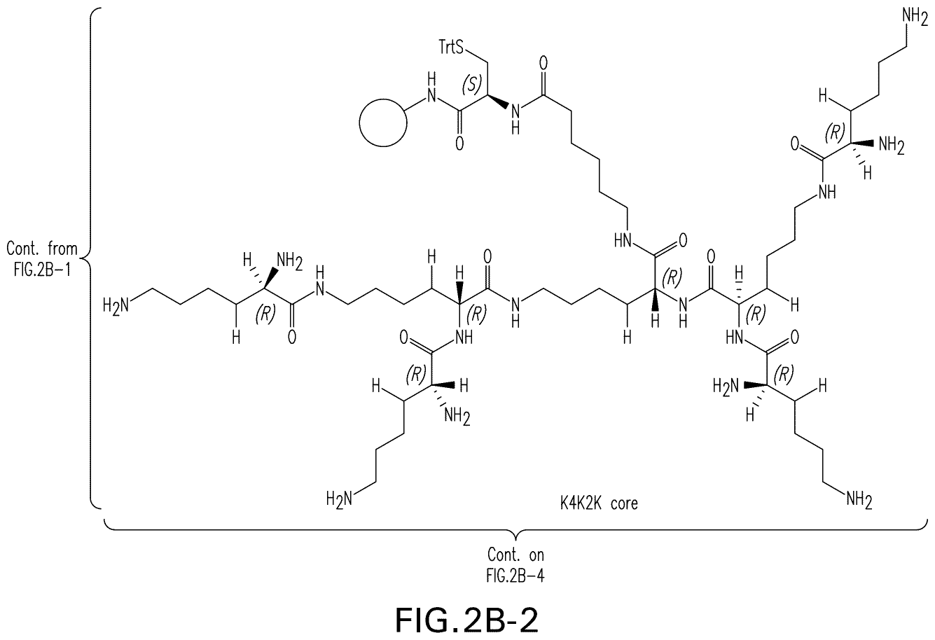

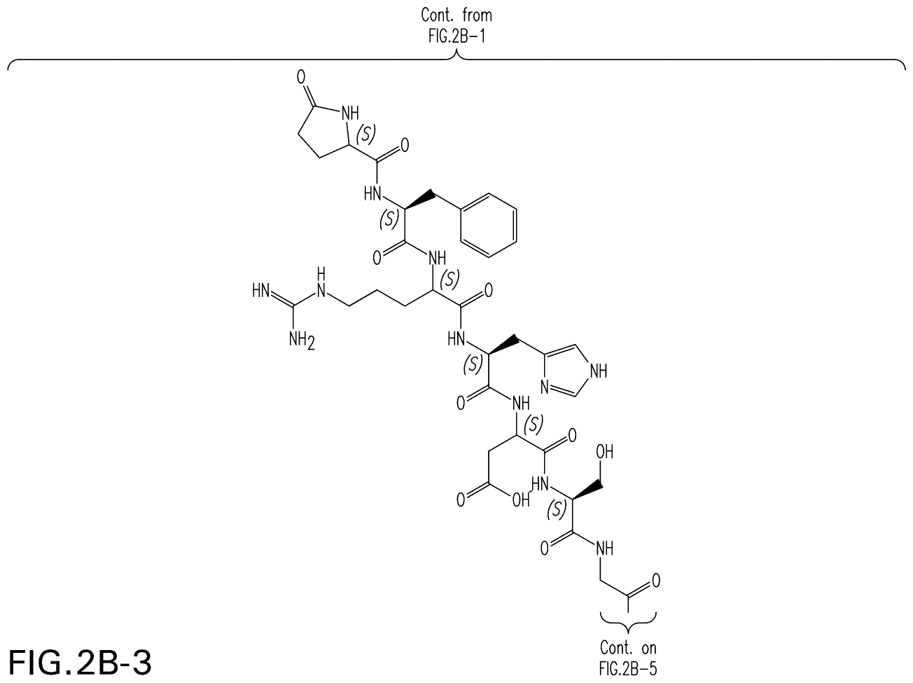

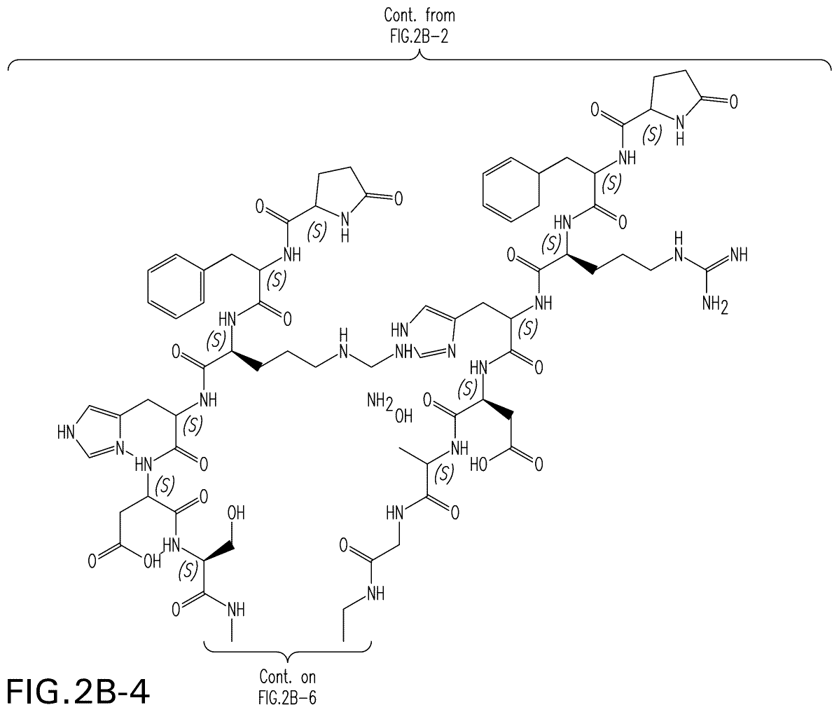

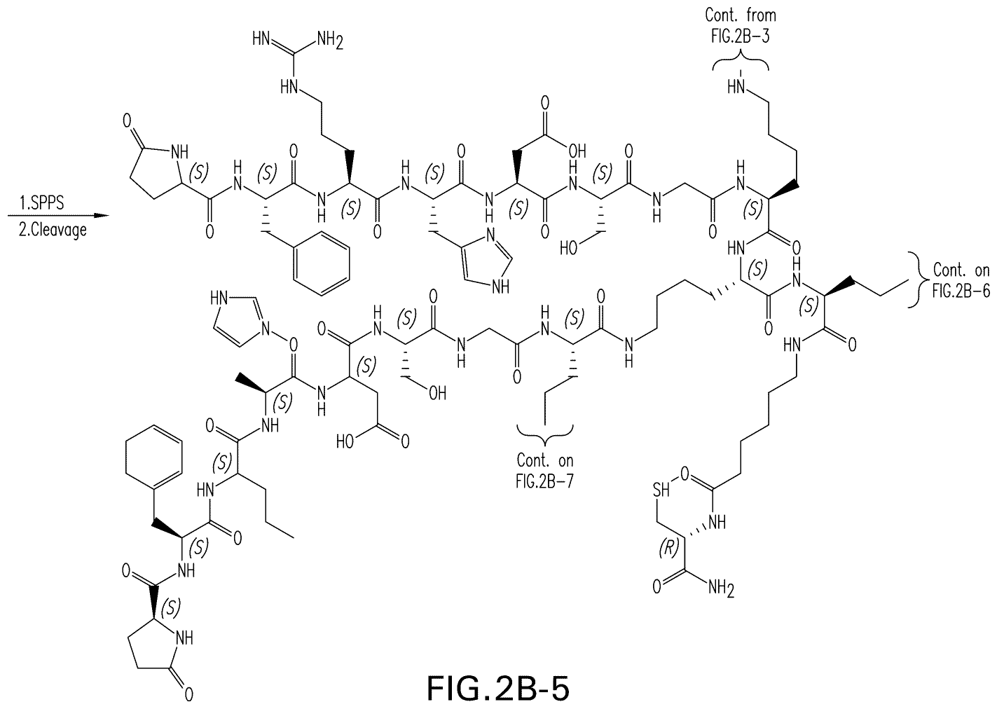

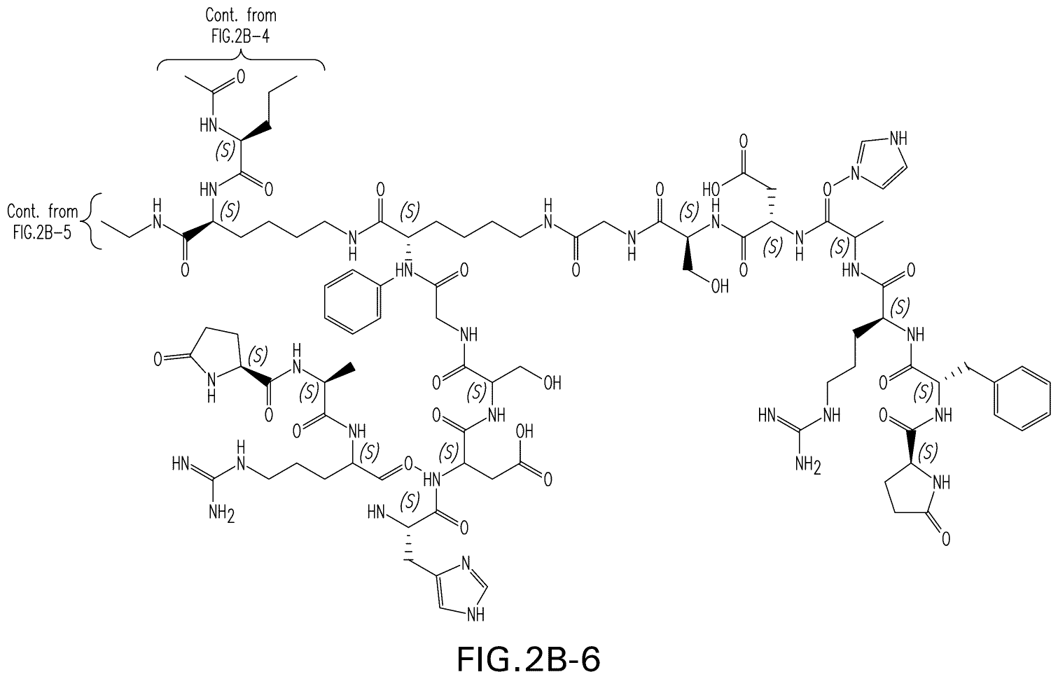

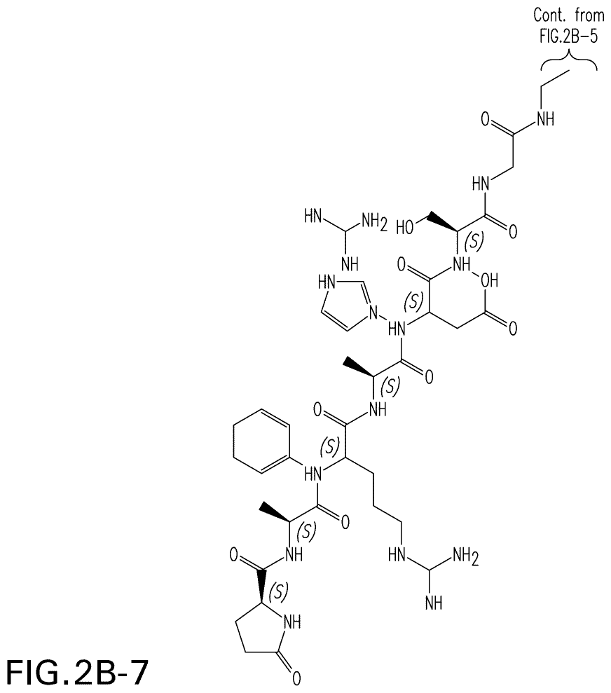

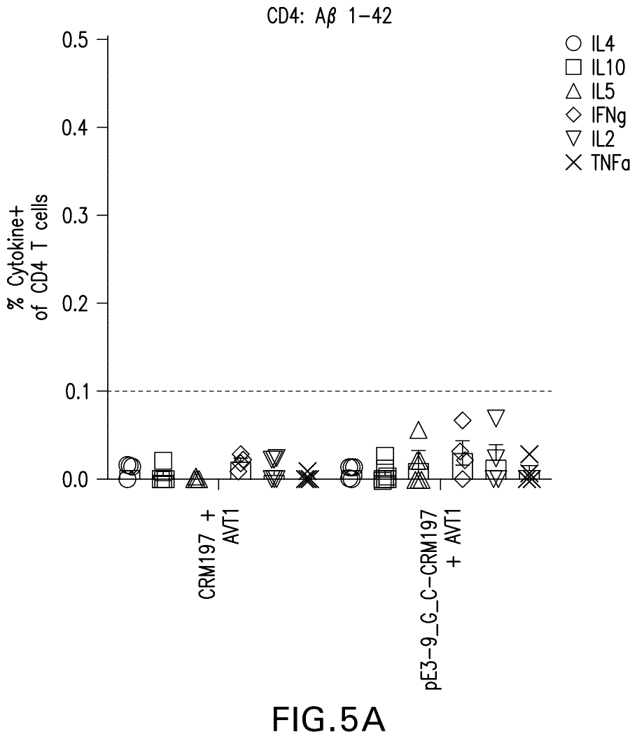

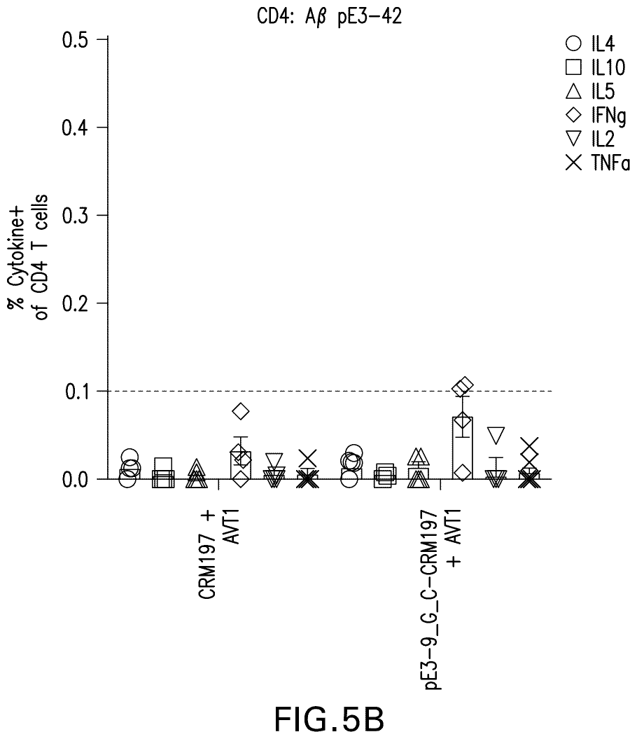

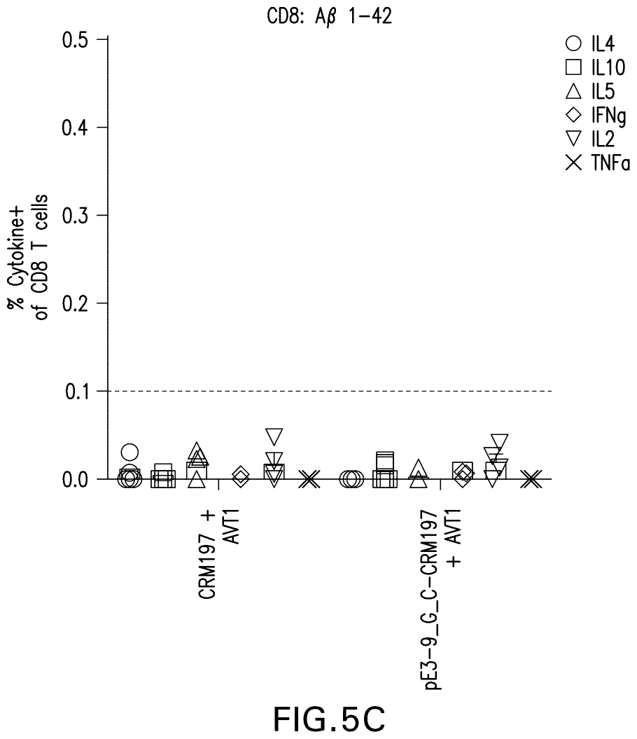

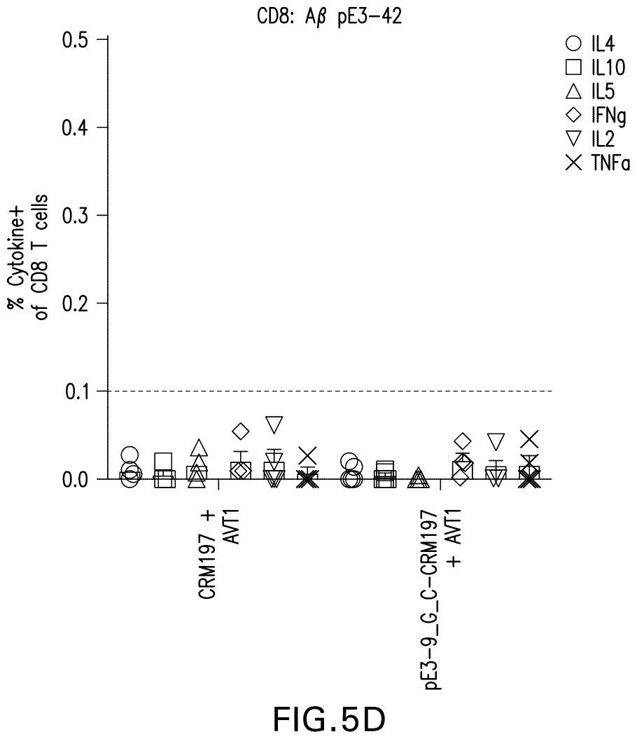

Examples of Aβ monomeric peptides that can be conjugated to carrier proteins, e.g., as monomers or multimers, are listed in Table 3 below. A particular pE 3-9_GC peptide (FIG. 1) tetramerized using PEG linkers is shown in FIG. 2A-1 through FIG. 2A-6. This peptide (SEQ ID NO:32) was treated to generate a free thiol group immediately before conjugation. The peptide (5.3 mg) was dissolved in 100 μl DMSO then added to 500 μl of 1N hydroxylamine solution, pH7.4. De-acylated peptide with an m/z of 1668.4 (+4) was detected immediately after mixing. The above peptide solution was further diluted 5× with conjugation buffer to have a hydroxylamine concentration of less than 0.1 N during conjugation.

Another multimeric peptide (SEQ ID NO:35), shown in FIG. 2B-1 through FIG. 2B-7, was synthesized through SPPS on Rink amide MBHA Resin (0.2 mmol, 0.33 mmol/g) following manual synthesis (protocol A) procedures described above. Fmoc-Lys(Fmoc) was used to build a K4K2K core. The eight Aβ peptide sequences were assembled independently on each chain of the core. The completed resin was cleaved following procedures described above. HPLC purification of crude peptide was carried out on a YMC-GEL 10 μm C4-HG column (160 mm×25 mm) (GX-281) column, using linear gradients of buffer B in A (A: H2O (0.1% TFA in H2O), mobile phase B: CH3CN:IPA=1:2 (0.1% TFA), at a flow rate of 20 mL/min. The Gradient was 24% B-45% B in 40 min. Product was eluted at 26 min. Fractions containing product were pooled and lyophilized to obtained white solid (34 mg, 2.18% yield) with observed m/z as 1524.3 (+4); 1089.4 (+6).

TABLE 3
Aβ peptides for conjugation

| SEQ | | | Observed |
|---|---|---|---|
| ID | | | m/z |
| NO | Peptide Amino Acid Sequence | Peptide Name | (charges) |
| 14 | [pE]FRHDSG[Ahx]C | pE3-9_Ahx_Cys | 1045.15 |
| 15 | [pE]FRHDSGGC | pE3-9_G_C | 988.98 |
| 16 | [pE]FRHDSGY[Ahx]C | pE3-10_Ahx_Cys | 604.80 (+2) |
| 17 | [pE]FRHDSGYE[Ahx]C | pE3-11_Ahx_Cys | 669.60 (+2) |
| 18 | [pE]FRHDSGYEV[Ahx]C | pE3-12_Ahx_Cys | 719.05 (+2) |
| 19 | [pE]FRHDSGYEVH[Ahx]C | pE3-13_Ahx_Cys | 787.63 (+2) |
| 20 | [pE]FRHDSGYEVHH[Ahx]C | pE3-14_Ahx_Cys | 855.70 (+2) |
| 21 | [pE]FRHDSGYEVHHQ[Ahx]C | pE3-15_Ahx_Cys | 919.55 (+2) |
| 22 | [pE]FRHDSGYEVHHQ[PEG4][Ahx]C | pE3-15_PEG4_Ahx_Cys | 1043.4 (+2) |
| 23 | [pE]FRHDSGYEVHHQK[Ahx]C | pE3-16_Ahx_Cys | 983.80 (+2) |
| 24 | [pE]FRHDSGYEVHHQKL[Ahx]C | pE3-17_Ahx_Cys | 1040.25 (+2) |
| 25 | [pE]VHHQKL[Ahx]C | pE11-17_Ahx_Cys | 544.74 (+2) |
| 26 | [pE]VHHQKLGC | pE11-17_G_C | 516.60 (+2) |
| 27 | [pE]FRHDSGGK(BrAc) | pE3-9_K(BrAc) | 567.3 (+2) |
| 28 | [pE]FRHDSGYEVHH[Ahx]K(BrAc) | pE3-14_Ahx-K(BrAc) | 619.2 (+2) |
| 29 | [pE]FRHDSGGAHIVMVDAYKPTK | pE3-9_G-SpyTag | 780.6 (+3) |
| 30 | [pE]FRHDSG[Ahx]AHIVMVDAYKPTK | pE3-9_Ahx-SpyTag | 799.5 (+3) |
| 31 | [pE]FRHDSG[PEG4]AHIVMVDAYKPTK | pE3-9_pEG4-SpyTag | 844.1 (+3) |
| and | | | |
| 41 | | | |
| 32 | {[pE]FRHDSGC(NH₂)[PEG]}4-PEG- | tetramer pE3-9_G-PEG | 1677.46 (+4) |
| | S(Ac) | linker-S(Ac) | |
| 33 | {[pE]FRHDSG}2K[Ahx]C | dimer pE3-9-Ahx-Cys | 992.2 (+2); |
| | | | 662.0 (+3) |
| 34 | {[pE]FRHDSG}4K2K[Ahx]C | tetramer pE3-9-Ahx-Cys | 966.2 (+4); |
| | | | 1287.8 (+3) |
| 35 | {[pE]FRHDSG}8K4K2K[Ahx]C | octamer pE3-9-Ahx-Cys | 1524.3 (+4); |
| | | | 1089.4 (+6) |

### Example 3: Preparation of AB Peptide-CRM197 Conjugates

Purified recombinant CRM197 (detoxified diphtheria toxin) was diluted to 1 mg/mL total protein concentration with 25 mM HEPES, pH 7.3, 150 mM NaCl, 5 mM EDTA. The CRM197 was activated with the heterobifunctional crosslinker N-maleimidobutyryloxysuccinimide ester, GMBS (TCI America, Portland, OR, USA). A stock solution of 30 mg/mL GMBS was freshly prepared in DMSO (Sigma Aldrich, D2650), and 50 equivalents of GMBS were added to the 1 mg/mL CRM197 solution. The reaction mixture was incubated at ambient temperature for two hours. Excess GMBS was removed by gel filtration using a disposable Sephadex® G25 desalting column (Cytiva). The level of maleimide in the GMBS-activated CRM197 was measured by consumption of N-acetylcysteine by the CRM197 bound maleimide, with the N-acetylcysteine thiol determined using Ellman's reagent (5,5-dithio-bis-(2-nitrobenzoic acid)).

Synthetic Aβ peptides, e.g., the Aβ peptides listed in Table 3 above, were dissolved in 20% DMSO at 10 mg/mL. A volume of the peptide solution was added to the GMBS-activated CRM197 so that the final conjugation reaction thiol to maleimide molar ratio was equal to 1.5. The reaction mixture was incubated at 4° C. for 16 hours. Unreacted maleimide groups were capped by addition of an excess of N-acetyl cysteine. A GMBS-activated CRM197-only control was carried through the conjugation protocol in parallel. The conjugate was purified through five successive rounds of centrifugal filtration in 30,000 dalton molecular weight cut-off filters with 25 mM HEPES, pH 7.3, 150 mM NaCl. The purified conjugate was sterile filtered with a 13 mm 0.2 μm Durapore® PVDF filter unit.

Analysis of Aβ peptide-CRM197 conjugates. The conjugate molecular weight was determined by size exclusion chromatography with multi-angle light scattering (MALS), differential refractive index (dRI) and UV detection at 280 nm. 50 μL of the peptide conjugate or control (GMBS-activated CRM197 quenched with N-acetyl cysteine) was injected on a Superose® 6 Increase 10/300 GL column (Cytiva, part no. GE29-0915-96) equilibrated with PBS+0.02% sodium azide at a flow rate of 0.5 mL/min. The molecular weight of the eluted peak was calculated by the Wyatt Astra® software. The peptide loading ratio (moles of peptide/moles CRM197) was calculated as the difference in molecular weight of the peptide-CRM197 conjugate and the CRM197-only control, divided by the molecular weight of the peptide. Peptide loading ratios for the conjugate ranged from 8.5 to 13.9 moles of peptide/mole of CRM197, with an average loading ratio of 11.4 moles of peptide/mole.

Alternatively, the molar ratio of peptide to protein was calculated by quantitative comparison of the experimentally determined amino acid composition of the conjugate and control (without peptide) using the modified least-squares algorithm described by Shuler, et al. (Shuler, K. R., Dunham, R. G., and Kanda, P. J. Immunol. Methods, 156 (1992), 137-149). The mass loading of peptide could be calculated from this ratio using the peptide molecular weight and the amino acid sequence-derived molecular weight for CRM197 (58,408 Da).

### Example 4: Preparation of AB Peptide-OMPC Conjugates

Maleimide chemistry. Outer membrane protein complex (OMPC) purified from Neisseria meningitidis was suspended in 50 mM NaHCO₃, pH 8.5 at 5 mg/mL. Freshly prepared GMBS at 30 mg/mL in 20% DMSO was added to the OMPC to give a molar ratio of GMBS to lysing of ca. 1.2 (assuming 0.42 pmole lysine/mg OMPC Lowry protein [Leanza, W. J., Chupak, L. S., Tolman, R. L., and Marbug, S. Bioconjug Chem 3(1992), 514-518]). The reaction was incubated for 2 hours at room temperature. The reaction mixture was desalted by dialysis against 25 mM HEPES, pH 7.3, 150 mM NaCl, 5 mM EDTA using a 100,000 dalton molecular cut-off dialysis membrane. The level of maleimide in the GMBS-activated OMPC was measured by consumption of N-acetylcysteine by the OMPC bound maleimide, with the N-acetylcysteine thiol determined using Ellman's reagent (5,5-dithio-bis-(2-nitrobenzoic acid)).

Synthetic Aβ peptides were dissolved in 20% DMSO at 10 mg/mL. A volume of the peptide solution was added to the GMBS-activated OMPC so that the final conjugation reaction thiol/maleimide molar ratio was equal to 1.5. The reaction mixture was incubated at 4° C. for 16 hours. Unreacted maleimide groups were capped by addition of an excess of N-acetyl cysteine. The conjugate was purified by dialysis against 25 mM HEPES, pH 7.3, 150 mM NaCl using a 100,000 Da molecular weight cut-off dialysis membrane.

The molar ratio of peptide to protein was calculated by quantitative comparison of the experimentally determined amino acid composition of the conjugate and control (without peptide) using the modified least-squares algorithm described by Shuler, et al. (Shuler, K. R., Dunham, R. G., and Kanda, P. J. Immunol. Methods, 156 (1992), 137-149). The peptide loading ratio of the conjugate was 4464 moles of peptide per mole of OMPC. Peptides conjugated to maleimide-activated OMPC are listed in Table 4 below.

TABLE 4
Peptides conjugated to maleimide-activated OMPC

| SEQ | Peptide Amino | |
|---|---|---|
| ID NO | Acid Sequence | Peptide Name. |
| 15 | [pE]FRHDSGGC | pE3-9_G_C |
| 20 | [pE]FRHDSGYEVHH[Ahx]C | pE3-14_Ahx_Cys |

Thiol derivatized OMPC. An alternative chemistry for conjugation involves reacting A3 peptides containing a terminal bromoacetyl functional group with thiol-derivatized OMPC. Purified, sterile OMPC was thiolated on a portion of its surface-accessible lysine residues using the reagent N-acetylhomocysteinethiolactone, NAHT (Sigma Aldrich, St. Louis, MO). OMPC in water was pelleted by centrifugation at approximately 197,000×g for 60 minutes at 4° C. and the supernatant was discarded. N₂-sparged activation buffer (0.11 M sodium borate, pH 11) was added to the centrifuge tube and the pellet was dislodged with a glass stir rod. The suspension was transferred to a glass Dounce homogenizer and resuspended with thirty strokes. The centrifuge tube was washed and the wash was dounced with thirty strokes. Re-suspended pellet and wash were combined in a clean vessel to give an OMPC concentration of approximately 7.8 mg/mL. Solid EDTA and DTT were dissolved in N₂-sparged activation buffer and added to the reaction at a ratio of 0.106 mg DTT/mg OMPC and 0.57 mg EDTA/mg OMPC. After gentle mixing, NAHT was dissolved in N2-sparged water and charged to the reaction at the ratio of 0.89 mg of NAHT/mg OMPC. The reaction proceeded for three hours at ambient temperature, protected from light. At completion, OMPC was pelleted as described above and re-suspended by dounce homogenization in N₂-sparged conjugation buffer (25 mM sodium borate, pH 8.5, 0.15 M NaCl). An aliquot was removed for free thiol determination by Ellman assay and the bulk product was stored on ice in the dark until use.

Bromoacetylated peptide was dissolved in N₂-sparged conjugation buffer at 10 mg/mL and slowly added to the thiolated OMPC solution. The reactions were protected from light and incubated at ambient temperature for approximately 16 hours. Residual free OMPC thiol groups were quenched with a 5-fold molar excess of N-ethylmaleimide for one hour at ambient temperature. A thiolated OMPC-only control was carried through the conjugation protocol in parallel. Upon completion of quenching, the conjugate and control were transferred to 100,000 Da molecular weight cut-off dialysis units and dialyzed exhaustively against at least five changes of conjugation buffer. The final purified conjugates were stored at 4° C. in sterile polypropylene tubes.

Analysis of Aβ peptide-OMPC conjugates. Total protein was determined by the modified Lowry assay and samples of conjugate and control were analyzed by quantitative amino acid analysis (AAA). The OMPC-specific concentration was determined from hydrolysis-stable residues which were absent from the peptide sequence and thus unique to OMPC protein. The molar ratio of peptide to protein was calculated by quantitative comparison of the experimentally determined amino acid composition of the conjugate and control (without peptide) using the modified least-squares algorithm described by Shuler, et al. (Shuler, K. R., Dunham, R. G., and Kanda, P. J. Immunol. Methods, 156 (1992), 137-149). The peptide loading ratio of the conjugate was 3963 moles of peptide per mole of OMPC. The mass loading of the peptide could be calculated from this ratio using the peptide molecular weight and an average OMPC mass of 40,000,000 Da. Peptides conjugated to thiol-activated OMPC are listed in Table 5 below.

TABLE 5
Peptides conjugated to thiol-activated OMPC

| SEQ | Peptide Amino | |
|---|---|---|
| ID NO | Acid Sequence | Peptide Name |
| 27 | [pE]FRHDSGGK[BrAc] | pE3-9_BrAc |
| 28 | [pE]FRHDSGYEVHH[Ahx]K[BrAc] | PE3-14_BrAc |

### Example 5: Preparation of Ab Peptide-KLH Conjugates

A 4 mg/ml suspension of mariculture keyhole limpet hemocyanin (mcKLH) was prepared by adding water to Imject PEGylated maleimide activated mcKLH (ThermoFisher Scientific Waltham, MA, USA). 0.5 ml of the mcKLH suspension was added directly to 2 mg of pE3_9-G-C solid peptide, and the peptide and activated mcKLH were gently mixed and allowed to react at room temperature overnight. The following day, the mixture was centrifuged at 3000 rpm to remove any precipitate formed during the reaction. The supernatant was transferred to a Slide-A-Lyzer 20k MWCO dialysis cassette (ThermoFisher Scientific, Waltham, MA, USA) for dialysis against PBS buffer. The retentate was collected and sterilized by filtering through a 0.2 μm Millex filter unit (Millipore Corp, Bedford, MA, USA). The amount of peptide incorporated into the conjugate was estimated by amino acid analysis following a 70-hour acid hydrolysis. Peptide concentration was determined to be 0.17 mg/ml.

A negative control was prepared by adding 5 μl of 0.5M N-acetyl Cysteine solution to 0.5 ml of the above activated mcKLH suspension. After an overnight reaction at room temperature, the mixture was centrifuged at 3000 rpm, and the supernatant was transferred to a 20k MWCO dialysis cassette for dialysis against PBS buffer. The retentate was collected and sterilized by filtering through a 0.2 u filter unit. Peptides conjugated to maleimide activated KLH are listed in Table 6 below.

TABLE 6
Peptides conjugated to maleimide activated KLH

| SEQ | Peptide Amino | |
|---|---|---|
| ID NO | Acid Sequence | Peptide Name |
| 15 | [pE]FRHDSGGC | pE3-9_G_C |

### Example 6: Preparation of Ab Peptide-VLP Conjugates

Aβ peptides were conjugated to virus-like particles (VLPs) of bacteriophage AP205 coat proteins using the SpyTag/SpyCatcher system. A 1 mg/mL solution of Aβ peptide containing a SpyTag sequence (SEQ ID NO:36) in 25 mM HEPES pH 7.3, 150 mM NaCl, was prepared. A 0.5 mg sample of AP205-SpyCatcher VLP (SEQ ID NO:37) was added to the peptide in a 1:1 molar ratio and allowed to react at 4° C. for 4 hours. The SpyTag and SpyCatcher sequences are shown in Table 7 below.

TABLE 7
SpyTag and SpyCatcher sequences

| SEQ | | |
|---|---|---|
| ID NO | Name | Amino Acid Sequence |
| 36 | SpyTag | AHIVMVDAYKPTK |
| 37 | SpyCatcher | GAMVDTLSGLSSEQGQSGDMTIEEDSATHI |
| | | KFSKRDEDGKELAGATMELRDSSGKTISTW |
| | | ISDGQVKDFYLYPGKYTFVETAAPDGYEVA |
| | | TAITFTVNEQGQVTVNGKATKGDAHI |

The conjugate was separated from unreacted peptide by buffer exchange using a 30 kDa Amicon spin filter (Millipore Corp, Bedford, MA, USA). The conjugate was sterilized by filtering through a 0.2 μm filter unit (Millipore Corp, Bedford, MA, USA) The amount of peptide incorporated into the conjugate was estimated by reduced time-of-flight mass spectrometry. Peptide concentrations were determined to be between 20 and 40 μg/mL. Peptides conjugated to VLPs are listed in Table 8 below.

TABLE 8
VLP-based conjugates

| SEQ | Peptide Amino | |
|---|---|---|
| ID NO | Acid Sequence | Peptide Name |
| 38 | [pE]FRHDSGGAHIVMVDA | pE3-9_G-SpyTag |
| | YKPTK | |
| 39 | [pE]FRHDSG[Ahx]AHIV | pE3-9_Ahx-SpyTag |
| | MVDAYKPTK | |
| 40 and | [pE]FRHDSG[PEG4]AHI | pE3-9_PEG4-SpyTag |
| 41 | VMVDAYKPTK | |

Mass analysis. For reduced mass analysis, 0.1 mg/mL Aβ peptide-VLP conjugates were prepared in 25 mM 1,4-dithiothreitol (DTT) solution. The samples were incubated at 50° C. for 10 minutes. The samples (0.5 μg/sample) were injected onto an Agilent 6230 LC-QToF-MS system (capillary voltage: 5K; desolvation temperature: 400° C.) equipped with an Agilent PLRP-S column (1000A 5 μm, 2.1×50 mm). Water with 0.1% (v/v) formic acid and acetonitrile with 0.1% (v/v) formic acid were used as the mobile phase A and B, respectively. The analysis was carried out at 60° C. with a gradient shown in Table 9 below.

TABLE 9
Mass analysis gradient conditions

| Time (min) | Flow Rate (ml/min) | % A | % B |
|---|---|---|---|
| 0 | 0.5 | 95 | 5 |
| 2 | 0.5 | 95 | 5 |
| 2.1 | 0.5 | 80 | 20 |
| 4 | 0.5 | 50 | 50 |
| 5 | 0.5 | 5 | 95 |
| 6 | 0.5 | 5 | 95 |
| 6.1 | 0.5 | 95 | 5 |
| 7 | 0.5 | 95 | 5 |

### Example 7: Preparation of Adjuvants

Aluminum Hydroxide. Amorphous aluminum hydroxyphosphate sulfate adjuvant (AAHS) was prepared by precipitation of alum with sodium hydroxide. Adjuvant concentration was calculated based on aluminum (Al³⁺) content. The aluminum concentration in each vaccine formulations was 450 μg/ml. The final aluminum dose per injection in animal studies was 45 μg in a volume of 100 μl.

Alum+CpG (type B ODN 1018). Amorphous aluminum hydroxyphosphate sulfate adjuvant (AAHS) was prepared by precipitation of alum with sodium hydroxide. Adjuvant concentration was calculated based on aluminum (Al³⁺) content. CpG (type B ODN 1018, TriLInk) was dissolved in 20 mM histidine, 100 mM NaCl, pH 6.5 buffer. The stock CpG solution was then filtered through 0.22 um PVDF membrane filter (4 mm diameter). AAHS was buffer exchanged into 20 mM histidine, 100 mM NaCl, pH 6.5 and concentrated via centrifugation. CpG 1018 was added to AAHS in the formulation buffer and mixed by pipetting. The aluminum concentration in each of the vaccine formulations was 450 μg/ml. The final aluminum dose per injection in animal studies was 45 μg Al³⁺ and 5 μg CpG or 30 μg CpG in a volume of 100 μl.

Liposomes. Liposomes containing QS-21, a plant extract from Quillaja saponaria (Desert King International, product 120-2-177), 1,2-dioleoyl-sn-glycero-3-phosphocholine (Avanti™, product 850375), cholesterol (Avanti™, product 700100), and GLA were formulated using thin film evaporation. DOPC, cholesterol, and GLA were dissolved in ethanol. The ethanol was evaporated in a round bottom flask using a RotoVap™. The lipid film was resuspended in 50 mM Na/K Phosphate, 100 mM NaCl pH 6.2 buffer over a water bath set to 50° C. The resultant liposomes were extruded 6 times through a 0.1 μm polycarbonate filter at 50° C. QS-21 and additional 50 mM Na/K Phosphate, 100 mM NaCl pH 6.2 buffer was added to the formulation, and subsequently sterile filtered through a PES 0.2 μm pore size membrane. In certain embodiments, AVT5 is an exemplary liposome adjuvant comprising DOPC, cholesterol, GLA, and QS-21 components.

AS04-like; TLR4+Alum. The TLR4 agonist GLA was dissolved in 0.2% triethylamine. It was heated, homogenized, and sonicated to fully dissolve and create uniformly sized particles. The particles were then filtered through a 0.22 μm PES sterile filter. The filtered GLA was then added to AAHS in 10 mM histidine, 325 mM NaCl, 0.01% PS80, pH 6.2. The formulation was allowed to equilibrate at 2-8° C. for at least 4 hours to ensure GLA was fully adsorbed to the AAHS. A final buffer exchange with 10 mM histidine, 325 mM NaCl, 0.01% PS80, pH 6.2 was performed. The supernatant and washes were collected to quantify the amount of, if any, unbound GLA.

Matrix-M like. QS-21 was dissolved into 150 mM NaCl, 10 mM K/Na Pi pH 6.2. DPPC and cholesterol were dissolved into 20% N-decanoyl-N-Methylglucamine and pipette mixed. Dissolved QS-21 was added to the lipid vial and mixed by pipetting and gentle vial swirling. 150 mM NaCl, 10 mM K/NA Pi pH 7.4 buffer was added and mixed. Stir overnight at 30° C. Dialyze against 150 mM NaCl, 10 mM K/NA Pi pH 7.4 for 24 hours, followed by 3× buffer exchanges into 150 mM NaCl, 10 mM K/Na Pi pH 6.2 24 hours apart. Sterile filter through a 0.22 μm pore size PES filter. The final dose per injection in animal studies was 0.2 μg or 5 μg of GLA in a volume of 100 μL.

SNE+L608. Squalene, PS-20, SPAN-85, and L608 ((12Z,15Z)-N,N-dimethyl-2-nonylhenicosa-12,15-dien-1-amine), as described in WO2017070623A1, the contents of which is incorporated herein in its entirety, were combined to allow for a target concentration of 15, 1.5, 1.5 and 15 mg/mL, respectively to create an oil phase. Aqueous buffer (20 mM histidine, pH 5.8) was added to the oil phase and mixed on a stir plate. A two-step high pressure homogenization process was employed, followed by sterile filtration with a PES 0.22 μm pore filter (catalog 12992). The filtered formulation was diluted with a pH 5.8 buffered combination of 20 mM histidine, 300 mM NaCl, and 0.2% PS-20 to achieve a final SNE adjuvant formulation targeting 2.4 mg/mL of L608.

SNE+TLR7/8. TLR7/8 agonist compounds of the Formula I, Ia, II, IIa, III, IIIa, IV or Iva, or pharmaceutically acceptable salt(s) thereof, are described in International Patent Application No. PCT/US2024/029596, the contents of which is incorporated herein in its entirety. In some embodiments, the TLR7/8 agonist compound is N-(5-(4-(4-((5-amino-7-(butylamino)-2H-pyrazolo[4,3-d]pyrimidin-2-yl)methyl)-3,5-dimethoxyphenyl)piperazin-1-yl)-5-oxopentyl)stearamide, N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide, or N-(4-((4-((5-amino-7-(butylamino)-2H-pyrazolo[4,3-d]pyrimidin-2-yl)methyl)-3,5-dimethoxybenzyl)(methyl)amino)butyl)stearamide. In certain exemplary embodiments, the TLR7/8 agonist compound is N-(5-(4-(4-((5-amino-7-(butylamino)-2H-pyrazolo[4,3-d]pyrimidin-2-yl)methyl)-3-methoxyphenyl)piperazin-1-yl)-5-oxopentyl)stearamide. The TLR7/8 agonist compounds were combined with Squalene, PS-20 or PS-80, and SPAN-85, and diluted with a pH 5.8 buffered combination of Histidine, PS-20, NaCl, L-met, and EDTA to create SNE adjuvant formulations.

LNPs. LNPs containing L608, DSPC, cholesterol, and PEG-DMG were formed by T-mix precipitation. The lipids were dissolved at a molar ratio of 58/10/30/2 in ethanol to generate AVT1, an exemplary LNP adjuvant. A syringe pump was used to introduce the lipid/ethanol stream to the aqueous stream (10 mM citrate buffer, pH 5) in a t-mix configuration. Two downstream buffer dilutions were introduced to the product stream (20 mM sodium citrate, 300 mM NaCl, pH 6.0 and 1× Dulbecco's PBS. The resultant LNPs were annealed at room temperature for 30 minutes before tangential film filtration for buffer exchange (20 mM tris, 10% sucrose, pH 7.5) and concentration. Aliquots are stored at −20° C. until the day of animal studies, where they are allowed to thaw at room temperature and diluted into 20 mM tris, 10% sucrose, pH 7.5.

### Example 8: In Vivo Immunogenicity Study

Immunogenicity of the peptides were tested by preparing a vaccine formulation comprising a peptide antigen conjugated to a carrier protein/particle that is co-formulated with a vaccine adjuvant. Peptide antigens were conjugated to a carrier protein or particle chosen from CRM197, KLH, OMPC, or AP205 virus-like particles (VLPs). The conjugated peptides were co-formulated with an adjuvant described in Example 7.

Mouse in vivo study protocol. Female C57Bl6/NTac mice (Taconic Biosciences), arrived in the facility and were group housed on corn cob bedding, 4 per cage with ad lib access to food and water under a normal light cycle (lights on 0700 h, lights off 1900 h). Mice arrived at 8 weeks of age and were habituated to the facility for at least a week prior to study initiation, when mice were between 9-11 weeks old. On Day 0 mice were vaccinated intramuscularly with 0.05 mL vaccine per quadricep, for a total infusion volume of 0.1 mL per mouse. Treatment groups were comprised of 8 mice, and animals were ear tagged for identification purposes. On day 15, mice were placed in warming boxes to promote tail vessel dilation and were tail bled with blood collected into a serum separator tubes (SSTs), which were then centrifuged at 10000 rpm for 6 minutes, with ˜100 μL of serum transferred to a 96 well plate according to a plate map. On Day 28, mice received a second vaccination with the same formulation as had been administered on Day 0 (0.05 mL IM per quadricep, for a total infusion volume of 0.1 mL per mouse). On Day 42, mice were euthanized with CO₂, and CSF was collected and transferred into a 96 well plate according to a plate map. Chest cavities were opened blood was collected into SSTs (and processed and plated as described on Day 15), spleens were dissected out and placed into collection vials kept on wet ice, and then mice were transcardially perfused with ice cold phosphate buffered saline via syringe. Brains were removed, hemisected and fresh frozen on dry ice. Tissues were then processed as described in this application.

Rhesus macaque in vivo study protocol. All animal studies were carried out at the New Iberia Primate Research Center (NIRC) (New Iberia, LA) and were approved by an Institutional Animal Care and Use Committee (IACUC). Healthy adult Indian-origin rhesus macaques of either sex were used for these studies. Prescreening of animals was conducted prior to study enrollment and animals were assigned to experimental groups with the goal of minimizing differences according to animal age, sex, body weight, and pre-existing immunity to vaccine components.

Animals were vaccinated according to established NIRC procedures which include sedation, shaving of injection site, and injection of test vaccine materials. Vaccines were delivered via the intramuscular (IM) route in the deltoid muscle with each arm receiving 0.5 ml of vaccine (1.0 ml total volume per animal). Animals were dosed at days 0 and 28 post-immunization and injection sites were monitors for Draize Score to determine reactogenicity. To evaluate immunogenicity of vaccine test materials venous blood was collected at appropriate time points longitudinally for isolation of serum, plasma, and peripheral blood mononuclear cells (PBMC). Serum was isolated from whole blood using appropriately sized non-additive serum separator vaccutainer-type tubes (SST), allowed to clot, centrifuged for serum harvest and transfer to long-term storage vials. Plasma was isolated from whole blood through collection into EDTA-containing tubes, samples were centrifuged, and plasma collected for long-term storage. PBMCs were isolated by collecting 30 ml of whole blood into appropriate EDTA-containing vacutainer tubes, processed by density gradient centrifugation according to NIRC standard operating procedures, and freezing prior to long-term storage.

### Example 9: Binding of Vaccine-Treated Animal Serum to AB Peptides

To determine immunogenicity of the vaccine formulation, mice were immunized two times, 4 weeks apart. Two weeks after each immunization, blood samples were collected, and serum tested by ELISA for antibody titers against Aβ_{pE3-42} and Aβ₁₋₄₂. These titers are shown in Table 11 below as post-dose 1 (PD1) and post-dose 2 (PD2) for Aβ_{pE3-42} and Aβ₁₋₄₂.

To further assess immunogenicity of the vaccine formulation, non-human primates were immunized two times, 4 weeks apart. Two weeks after each immunization, blood samples were collected, and serum tested by ELISA for antibody titers against Aβ_{pE3-42} and Aβ₁₋₄₂. These titers are shown in Table 12 for Aβ_{pE3-42} and Aβ₁₋₄₂.

Immunogenicity was assessed using Aβ_{pE3-42} titers, and specificity for Aβ_{pE3-42} was assessed by comparing titers for Aβ_{pE3-42} versus Aβ₁₋₄₂. Assessment of binding of mouse or non-human primate (NHP) serum to Aβ peptides, Aβ_{pE3-42} and Aβ₁₋₄₂ was carried out by enzyme-linked immunosorbent assay (ELISA), following a protocol described in Example 3 of International Patent Application Publication WO2010005858A, the contents of which is incorporated herein in its entirety. Black 384-well immuno plates (ThermoFisher Scientific, Rochester, NY) were coated with 25 μL per well of either Aβ_{pE3-42} or Aβ₁₋₄₂ peptide at a concentration of 0.25 μg per mL in PBS and stored at 4° C. overnight. Vaccine treated animal serum was prepared in milk-PBST with an initial 1:50 serum dilution into milk-PBST and a subsequent 4-fold dilution series to achieve a 10-point, 4-fold titration of animal serum in milk-PBST across a serum block plate. Each individual animal serum sample was platted in this manner across a serum block plate.

Assay plates were then washed 6 times with PBS containing 0.05% Tween-20 (PBST) and blocked for 1 hour with 80 μL per well of 3% skim milk in PBST (milk-PBST). Plates were then washed once and 20 μL of serum was stamped from the serum block plate into the assay plate. Assay plate with serum in wells was incubated in a humidified incubator for 2 hours at 22° C. Assay plates were then washed 6 times with PBS containing 0.05% Tween-20 (PBST). Next, 20 μL of freshly prepared HRP-conjugated secondary antibody was applied to each well and incubated in a humidified incubator for 1 hour at 22° C. Antibodies used in this ELISA assay are shown in Table 10 below.

TABLE 10
ELISA antibodies

| Antibody | Source | Cat. No. | Storage |
|---|---|---|---|
| Goat anti-mouse IgG | ThermoFisher | A16084 | 4° C. storage |
| (Fc)total IgG, HRP | Scientific | Lot 65-123- | |
| conjugated secondary | | 031219 | |
| antibody, 1 mg/ml. | | | |
| Rat anti-mouse IgG1, HRP | Abcam | ab99603 | 4° C. storage |
| conjugated secondary | | Lot GR3267131-1 | |
| antibody, 500 μl | | | |
| Goat anti-mouse IgG2C, | Southern | 1079-05 | 4° C. storage |
| HRP conjugated secondary | Biotech | Lot F0919-W891 | |
| antibody, 1 ml | | | |
| Goat anti-human IgG | ThermoFisher | A18811 | Stored at 4° C. This |
| (Fc)total IgG, HRP | Scientific | | secondary antibody can |
| conjugated secondary | | | be used for NHP and |
| antibody, 1 mg/ml. | | | human sample detection. |

Assay plates were then move to open air an incubated or 10 mm at room PGP temperature. Assay plates were washed 6 times with PBS containing 0.05% Tween-20 (PBST). West Pico PLUS chemiluminescent substrate was prepared according to manufacturer protocol (ThermoFisher Scientific, Rochester, NY). 20 μL of freshly prepared chemiluminescent substrate was added to each well of the assay plates and incubated in open air at room temperature for 15 minutes. Luminescence was read on the EnVision Multimode plate reader (Perkin Elmer, Waltham, MA) set to read ultrasensitive luminescence at 0.1 second/well.

Interpolated ELISA titers were then calculated from plate reader raw data using a threshold of 50,000 relative luminescence units (RLU) counts. Titer was calculated as follows.

$$titer = ( starting dilution / series dilution factor ) \times ( series dilution {factor}^{∧} t )$$

$$where t = x - [ ( cut - off - L ) / ( H - L ) ]$$

$$H = High well count = the counts of the first well ABOVE the cutoff$$

$$L = Low well count = the counts of the first well BELOW the cutoff$$

$$x = Low well number = the column \# of the “ low well count ”$$

$$cut - off = 50 , 000 counts$$

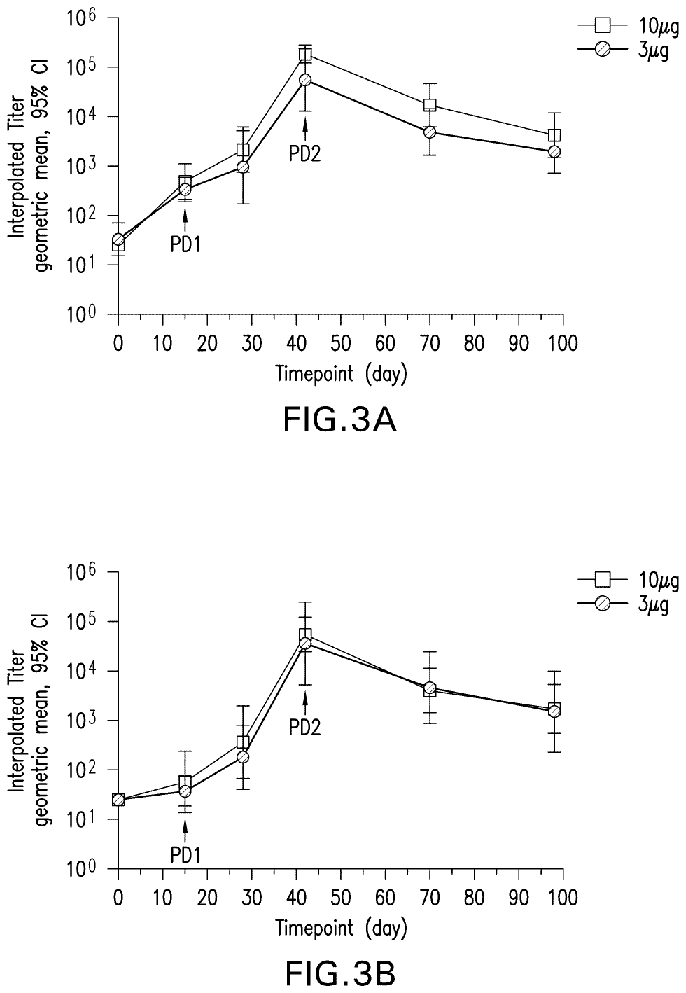

The interpolated titer is the calculated x dilution point at which the line crosses 50,000 RLU between the data points one above and below the 50,000 RLU threshold. Samples where no dilution crosses the threshold are given a placeholder titer of “25”. Results of this assay are summarized in Tables 11 and 12 below and in FIG. 3A and FIG. 3B. The graphs plot individual animal data, a geometric mean of data points per vaccine treatment group and a 95% CI.

TABLE 11
Summary of mouse titers for all vaccine conjugates.

| | | Total IgG | | | |
|---|---|---|---|---|---|
| | | Aβ pE3-42 | | Aβ 1-42 | |
| Aβ amino acid # | Construct | PD1 | PD2 | PD1 | PD2 |
| 3 ug pE3-9_Ahx | CRM197 + 125 ug AVT1 | 2689 | 605716 | 43 | 169 |
| 3 ug pE3-9_C_G | CRM197 + 125 ug AVT1 | 10688 | 2139854 | 101 | 1793 |
| 3 ug pE3-10_Ahx | CRM197 + 125 ug AVT1 | 595 | 547682 | 25 | 136 |
| 3 ug pE3-11_Ahx | CRM197 + 125 ug AVT1 | 5178 | 745517 | 996 | 10362 |
| 3 ug pE3-12-Ahx | CRM197 + 125 ug AVT1 | 1083 | 797421 | 172 | 3092 |
| 3 ug pE3-13_Ahx | CRM197 + 125 ug AVT1 | 3697 | 1103654 | 220 | 3773 |
| 3 ug pE11-17_Ahx | CRM197 + 125 ug AVT1 | 25 | 25 | 25 | 25 |
| 3 ug pE11-17_G | CRM197 + 125 ug AVT1 | 25 | 333 | 25 | 52 |
| 3 ug pE3-14_Ahx | CRM197 + 125 ug AVT1 | 10402 | 803649 | 198 | 3617 |
| 3 ug pE3-15_Ahx | CRM197 + 125 ug AVT1 | 6876 | 800133 | 160 | 6273 |
| 3 ug pE3-15_PEG4_Ahx | CRM197 + 125 ug AVT1 | 25194 | 678884 | 519 | 11693 |
| 3 ug pE3-16_Ahx | CRM197 + 125 ug AVT1 | 13184 | 918205 | 734 | 56128 |
| 3 ug pE3-17_Ahx | CRM197 + 125 ug AVT1 | 1093 | 368149 | 59 | 4889 |
| 3 ug pE3-9_G_C | CRM197 + PBS | 38 | 120 | 25 | 25 |
| 0.3 ug pE3-9_G_C | CRM197 + 25 ug AVT1 | 1365 | 847530 | 25 | 39 |
| 1 ug pE3-9_G_C | CRM197 + 25 ug AVT1 | 1644 | 596466 | 25 | 46 |
| 3 ug pE3-9_G_C | CRM197 + 25 ug AVT1 | 11051 | 476265 | 25 | 56 |
| 0.3 ug pE3-9_G_C | CRM197 + 125 ug AVT1 | 1336 | 870468 | 25 | 49 |
| 1 ug pE3-9_G_C | CRM197 + 125 ug AVT1 | 14612 | 680191 | 31 | 270 |
| 3 ug p pE3-9_G_C | CRM197 + 125 ug AVT1 | 55318 | 928637 | 27 | 254 |
| 0.3 ug pE3-9_G_C | CRM197 + 45 ug MAA | 43 | 18513 | 25 | 25 |
| 1 ug pE3-9_G_C | CRM197 + 45 ug MAA | 91 | 76278 | 25 | 25 |
| 3 ug pE3-9_G_C | CRM197 + 45 ug MAA | 137 | 89375 | 25 | 28 |
| 3 ug pE3-9_G_C + 0.1% PS80 | CRM197 + 125 ug AVT1 | 18558 | 2123821 | 39 | 216 |
| Spike (Shipped) | | | | | |
| 3 ug pE3-14_Ahx + 0.1% PS80 | CRM197 + 125 ug AVT1 | 1149 | 1321831 | 45 | 3467 |
| Spike (Shipped) | | | | | |
| 3 ug pE3-9_G_C + 0.1% PS80 | CRM197 + 125 ug AVT1 | 45027 | 1554270 | 66 | 57 |
| Spike (Not Shipped) | | | | | |
| 3 ug pE3-14_Ahx + 0.1% PS80 | CRM197 + 125 ug AVT1 | 8953 | 2473386 | 95 | 3226 |
| Spike (Not Shipped) | | | | | |
| 3 ug pE3-9_G_C + Sucrose Spike | CRM197 + 125 ug AVT1 | 34005 | 2427906 | 31 | 93 |
| in CRM197 (SHIPPED | | | | | |
| FROZEN) | | | | | |
| 3 ug pE3-14_Ahx + Sucrose Spike | CRM197 + 125 ug AVT1 | 9215 | 1349559 | 97 | 14079 |
| in CRM197 (SHIPPED | | | | | |
| FROZEN) | | | | | |
| 3 ug pE3-9_G_C + Sucrose Spike | CRM197 + 125 ug AVT1 | 36085 | 2196319 | 33 | 261 |
| (Not Shipped) | | | | | |
| 3 ug pE3-14_Ahx + Sucrose Spike | CRM197 + 125 ug AVT1 | 10218 | 1328536 | 55 | 2195 |
| (Not Shipped) | | | | | |
| 3 ug pE3-9_G_C Control (Not | CRM197 + 125 ug AVT1 | 48561 | 2372631 | 28 | 38 |
| Shipped) | | | | | |
| 3 ug pE3-14_Ahx Control (Not | CRM197 + 125 ug AVT1 | 5735 | 1792642 | 91 | 27179 |
| Shipped) | | | | | |
| 0.1 ug pE3-9_G_C | CRM197 + 45 ug MAA | 38 | 1274 | 25 | 25 |
| 1 ug pE3-9_G_C | CRM197 + 45 ug MAA | 180 | 125127 | 32 | 29 |
| 0.1 ug pE3-9_G_C | maOMPC + 45 ug MAA | 9176 | 183136 | 25 | 76 |
| 1 ug pE3-9_G_C | maOMPC + 45 ug MAA | 98623 | 4092869 | 38 | 2359 |
| 0.1 ug pE3-9_G_C | SH-OMPC + 45 ug MAA | 17123 | 311945 | 25 | 43 |
| 1 ug pE3-9_G_C | SH-OMPC + 45 ug MAA | 108705 | 2178541 | 28 | 32 |
| 0.1 ug pE3-14_Ahx | CRM197 + 45 ug MAA | 25 | 576 | 25 | 25 |
| 1 ug pE3-14_Ahx | CRM197 + 45 ug MAA | 59 | 15490 | 25 | 59 |
| 0.1 ug pE3-14_Ahx | maOMPC + 45 ug MAA | 5166 | 223286 | 112 | 370 |
| 1 ug pE3-14_Ahx | maOMPC + 45 ug MAA | 803 | 102773 | 221 | 5177 |
| 0.1 ug pE3-14_Ahx | SH-OMPC + 45 ug MAA | 24598 | 718389 | 1858 | 1166 |
| 1 ug pE3-14_Ahx | SH-OMPC + 45 ug MAA | 30008 | 2145759 | 435 | 3388 |
| 1 ug pE3-9_G_C | KLH + 45 ug MAA | 2115 | 24101 | 25 | 25 |
| 1 ug pE3-9_G_C | AP205 Prep B206 + 45 ug | 740 | 525 | 41 | 32 |
| | MAA | | | | |
| 1 ug pE3-9_G_C | AP205 Prep B208 + 45 ug | 1865 | 53413 | 25 | 46 |
| | MAA | | | | |
| 1 ug pE3-9_G_C | AP205 Prep B209 + 45 ug | 2720 | 140113 | 29 | 25 |
| | MAA | | | | |
| empty | KLH + 45 ug MAA | 25 | 25 | 31 | 31 |
| empty | AP205 + 45 ug MAA | 25 | 25 | 25 | 25 |
| 1 ug pE3-9_G_C | CRM197 + 45 ug MAA | 323 | 35260 | 25 | 36 |
| 100 ug pE3-9_G_C | none + 45 ug MAA (B185) | 25 | 25 | 25 | 25 |
| 100 ug pE3-9_G_C | none + 45 ug MAA (B186) | 25 | 230 | 25 | 25 |
| 0.3 ug pE3-9_G_C | CRM197 + 45 ug MAA | 43 | 19405 | 25 | 25 |
| 0.3 ug pE3-9_G_C | APA + 25 ug APA | 36 | 15666 | 25 | 25 |
| 0.3 ug pE3-9_G_C | APA + 50 ug APA | 33 | 3940 | 25 | 25 |
| 0.3 ug pE3-9_G_C | CRM197 + 0.2 ug AVT3 | 25 | 67 | 25 | 25 |
| 0.3 ug pE3-9_G_C | CRM197 + 5 ug AVT3 | 1035 | 2544686 | 38 | 53 |
| 0.3 ug pE3-9_G_C | CRM197 + 0.3 ug AVT4 | 251 | 243376 | 25 | 25 |
| 0.3 ug pE3-9_G_C | CRM197 + 30 ug AVT4 | 812 | 4234488 | 25 | 25 |
| 0.3 ug pE3-9_G_C | CRM197 + 40 ug AVT6 | 5033 | 1265022 | 25 | 35 |
| 0.3 ug pE3-9_G_C | CRM197 + 120 ug AVT6 | 2931 | 1691482 | 32 | 57 |
| 0.3 ug pE3-9_G_C | CRM197 + 1200 ug AVT8 | 4117 | 2266220 | 29 | 36 |
| 0.3 ug pE3-9_G_C | CRM197 + 2500 ug AVT8 | 1672 | 2173187 | 25 | 25 |
| 0.3 ug pE3-9_G_C | CRM197 + 45 ug MAA/5 ug | 140 | 333576 | 25 | 25 |
| | CpG | | | | |
| 0.3 ug pE3-9_G_C | CRM197 + 45 ug MAA/30 ug | 214 | 990128 | 25 | 126 |
| | CpG | | | | |
| 0.3 ug pE3-9_G_C | CRM197 + 0.15 ug AS01-like | 349 | 737665 | 25 | 35 |
| | TLR4 | | | | |
| 0.3 ug pE3-9_G_C | CRM197 + 0.5 ug AS01-like | 3858 | 4349916 | 25 | 65 |
| | TLR4 | | | | |
| 0.3 ug pE3-9_G_C | CRM197 + 0.2 ug AS04-like | 115 | 274826 | 25 | 32 |
| 0.3 ug pE3-9_G_C | CRM197 + 5 ug AS04-like | 865 | 516454 | 25 | 46 |
| 0.3 ug pE3-9_G_C | CRM197 + 45 ug MAA | 131 | 99117 | 25 | 32 |
| 0.3 ug pE3-9_G_C | CRM197 + 25 ug AVT1 | 4852 | 1572258 | 25 | 34 |
| 0.01 ug pE3-9_G_C | SH-OMPC + 45 ug MAA | 138 | 37090 | 25 | 25 |
| 0.03 ug pE3-9_G_C | SH-OMPC + 45 ug MAA | 186 | 207573 | 25 | 35 |
| 0.1 ug pE3-9_G_C | SH-OMPC + 45 ug MAA | 473 | 856938 | 25 | 60 |
| 0.3 ug pE3-9_G_C | SH-OMPC + 45 ug MAA | 997 | 2145870 | 25 | 32 |
| 1 ug pE3-9_G_C | SH-OMPC + 45 ug MAA | 3254 | 2736084 | 25 | 28 |
| 0.3 ug pE3-9_G_C | CRM197 + 45 ug MAA | 36 | 24377 | 25 | 25 |
| 1 ug pE3-9_G_C | CRM197 + 45 ug MAA | 36 | 40351 | 25 | 25 |

TABLE 12
NHP animal group geometric mean of interpolated ELISA titers

| | | Total IgG Aβ pE3-42 | | | | |
|---|---|---|---|---|---|---|
| Aβ amino acid # | Construct | Day 15 | Day 27 | Day 42 | Day 70 | Day 98 |
| 3 μg pE 3-9_G_C | 10 ug CRM197 + | 337 | 942 | 55430 | 4887 | 1966 |
| | 300 ug AVT1 | | | | | |
| 3 μg pE 3-9_G_C | 33.3 ug CRM197 + | 488 | 2123 | 184632 | 16875 | 4175 |
| | 300 ug AVT1 | | | | | |
| 3 μg pE 3-9_G_C | 100 ug CRM197 + | 682 | 1488 | 119514 | 11701 | 3012 |
| | 300 ug AVT1 | | | | | |
| 3 μg pE 3-14_Ahx | 10 ug CRM197 + | 38 | 181 | 35909 | 4617 | 1519 |
| | 300 ug AVT1 | | | | | |
| 10 μg pE 3-14_Ahx | 33.3 ug CRM197 + | 58 | 364 | 54786 | 4038 | 1704 |
| | 300 ug AVT1 | | | | | |
| 30 μg pE 3-14_Ahx | 100 ug CRM197 + | 341 | 598 | 42174 | 4004 | 1217 |
| | 300 ug AVT1 | | | | | |
| | | Total IgG Aβ 1-42 | | | | |
| Aβ amino acid # | Construct | Day 15 | Day 27 | Day 42 | Day 70 | Day 98 |
| 3 μg pE 3-9_G_C | 10 ug CRM197 + | 43 | 29 | 64 | 30 | 25 |
| | 300 ug AVT1 | | | | | |
| 10 μg pE 3-9_G_C | 33.3 ug CRM197 + | 35 | 50 | 73 | 25 | 25 |
| | 300 ug AVT1 | | | | | |
| 30 μg pE 3-9_G_C | 100 ug CRM197 + | 48 | 37 | 113 | 37 | 25 |
| | 300 ug AVT1 | | | | | |
| 3 μg pE 3-14_Ahx | 10 ug CRM197 + | 38 | 35 | 276 | 65 | 29 |
| | 300 ug AVT1 | | | | | |
| 10 μg pE 3-14_Ahx | 33.3 ug CRM197 + | 30 | 79 | 164 | 47 | 25 |
| | 300 ug AVT1 | | | | | |
| 30 μg pE 3-14_Ahx | 100 ug CRM197 + | 50 | 55 | 181 | 37 | 25 |
| | 300 ug AVT1 | | | | | |

| | | Total IgG Aβ pE3-42 | | | | | | | |
|---|---|---|---|---|---|---|---|---|---|
| Aβ amino acid # | Construct | Day 15 | Day 28 | Day 42 | Day 49 | Day 56 | Day 70 | Day 98 | Day 139 |
| 2.5 μg pE 3- | CRM197 + 500 ug | 460 | 1639 | 165029 | 67706 | 41744 | 26777 | 6520 | 1556 |
| 9_G_C | MAA | | | | | | | | |
| 2.5 μg pE 3- | CRM197 + 500 ug | 11966 | 7523 | 227575 | 79967 | 59319 | 38441 | 10429 | 2660 |
| 9_G_C | MAA | | | | | | | | |
| 0.5 μg pE 3- | CRM197 + 500 ug | 5016 | 4639 | 84092 | 39734 | 31590 | 18630 | 5473 | 1543 |
| 9_G_C | MAA | | | | | | | | |
| 0.1 μg pE 3- | CRM197 + 500 ug | 1965 | 2851 | 46681 | 23706 | 23780 | 14098 | 3606 | 845 |
| 9_G_C | MAA | | | | | | | | |
| 2.5 μg pE 3- | CRM197 + 10 mg | 5006 | 10066 | 475117 | 170443 | 134568 | 110628 | 34579 | 10992 |
| 9_G_C | SNE; MF59-like | | | | | | | | |
| 2.5 μg pE 3- | CRM197 + AS04- | 1047 | 3031 | 284474 | 102657 | 73631 | 50174 | 8207 | 1546 |
| 9_G_C | like; TLR4 + Alum | | | | | | | | |
| 2.5 μg pE 3- | CRM197 + AS01- | 3788 | 5540 | 586403 | 162070 | 117262 | 73497 | 14485 | 4513 |
| 9_G_C | like; TLR4 + QS-21 | | | | | | | | |
| 0.5 μg pE 3- | SH-OMPC + MAA + | 1527 | 10312 | 170333 | 87817 | 51456 | 54017 | 16991 | 6039 |
| 9_G_C | SNE + TLR7/8* | | | | | | | | |
| 0.5 μg pE 3- | SH-OMPC + AS04- | 25041 | 28284 | 537268 | 218693 | 135527 | 92450 | 19035 | 2409 |
| 9_G_C | like; TLR4 + Alum | | | | | | | | |
| 0.5 μg pE 3- | SH-OMPC + MAA + AS01- | 30290 | 32683 | 550915 | 472329 | 185799 | 157400 | 60166 | 20973 |
| 9_G_C | like; TLR4 + QS-21 | | | | | | | | |
| | | Total IgG Aβ 1-42 | | | | | | | |
| Aβ amino acid # | Construct | Day 15 | Day 28 | Day 42 | Day 49 | Day 56 | Day 70 | Day 98 | Day 139 |
| 2.5 μg pE 3- | CRM197 + 500 ug MAA | 25 | 25 | 120 | 55 | 53 | 47 | 33 | 25 |
| 9_G_C | | | | | | | | | |
| 2.5 μg pE 3- | CRM197 + 500 ug MAA | 25 | 25 | 77 | 37 | 39 | 41 | 25 | 25 |
| 9_G_C | | | | | | | | | |
| 0.5 μg pE 3- | CRM197 + 500 ug MAA | 25 | 25 | 57 | 39 | 41 | 40 | 33 | 25 |
| 9_G_C | | | | | | | | | |
| 0.1 μg pE 3- | CRM197 + 500 ug MAA | 25 | 25 | 38 | 25 | 25 | 32 | 25 | 25 |
| 9_G_C | | | | | | | | | |
| 2.5 μg pE 3- | CRM197 + 10 mg SNE; | 25 | 25 | 409 | 122 | 135 | 144 | 42 | 34 |
| 9_G_C | MF59-like | | | | | | | | |
| 2.5 μg pE 3- | CRM197 + AS04-like; | 25 | 31 | 105 | 41 | 43 | 53 | 35 | 25 |
| 9_G_C | TLR4 + Alum | | | | | | | | |
| 2.5 μg pE 3- | CRM197 + AS01-like; | 25 | 25 | 209 | 48 | 52 | 65 | 41 | 25 |
| 9_G_C | TLR4 + QS-21 | | | | | | | | |
| 0.5 μg pE 3- | SH-OMPC + MAA + | 25 | 25 | 100 | 41 | 38 | 63 | 32 | 25 |
| 9_G_C | SNE + TLR7/8* | | | | | | | | |
| 0.5 μg pE 3- | SH-OMPC + AS04-like; | 44 | 35 | 279 | 108 | 81 | 122 | 31 | 25 |
| 9_G_C | TLR4 + Alum | | | | | | | | |
| 0.5 μg pE 3- | SH-OMPC + MAA + AS01- | 62 | 33 | 199 | 66 | 94 | 126 | 36 | 25 |
| 9_G_C | like; TLR4 + QS-21 | | | | | | | | |

Analysis of CRM197 conjugated antigens using AVT1 adjuvant (a lipid nanoparticle adjuvant containing cationic lipid/cholesterol/DSPC/PEG-DMG in a molar ratio of 40/48/10/2, and prepared as described in WO2015/130584A2, the contents of which is herein incorporated by reference in its entirety) showed that immunogenicity and specificity for Aβ_{pE3-42} versus Aβ₁₋₄₂ can vary depending on antigen sequence and linker. Further analysis of the data of Tables 11 and 12 showed that the carrier protein or particle and adjuvant can also impact immunogenicity and specificity. The pE3-9-CRM197+AVT1 formulation represents a formulation with desired immunogenicity and selectivity for Aβ_{pE3-42} versus Aβ₁₋₄₂.

### Example 10: Rhesus PBMC Restimulation Assay and Intracellular Cytokine Staining

T cell responses following stimulation with Aβ_{pE3-42} or Aβ₁₋₄₂ were assessed to determine whether the vaccines induced a T cell response against Aβ_{pE3-42} or Aβ₁₋₄₂. Cryopreserved rhesus macaque PBMCs were quick-thawed in a 37° C. water bath and washed with R10 medium (RPMI-1640 supplemented with 10% fetal bovine serum (FBS), 10 mM HEPES buffer (ph7.2-7.5), 2 mM L-glutamine, 1× penicillin-streptomycin, 1 mM sodium pyruvate, and 50 μM 2-Mercaptoethanol). Cells were incubated overnight at 37° C., 5% CO₂. After overnight recovery, cells were counted and distributed into 96-well round bottom plates and cultured with peptides spanning the human Aβ₁₋₄₂ sequence (15 mers, overlapping by 14 amino acids), human Aβ_{pE3-42} sequence (15 mers, overlapping by 14 amino acids), or CRM197 protein (15 mers, overlapping by 11 amino acids) at 2 ug/ml and CD28/CD49d mouse anti-human costimulatory antibodies at 1.25 ug/ml. Plates were incubated at 37° C. for 60 minutes. Following incubation, freshly diluted brefeldin A was added at a final concentration of 12.5 ug/ml and the plates were incubated for an additional 5 hours. DMSO was added to mock control wells in place of peptide to determine background cytokine expression levels.

Following stimulation, PBMC were washed with phosphate-buffered saline (PBS) and stained with mouse anti-human CCR7-BV650 (clone G043H7, Biolegend) at 37° C. 10 minutes. Live/Dead fixable Aqua stain (Invitrogen) was added to each well and samples were incubated for 15 minutes at room temperature. Cells were washed with FACS buffer (PBS with 1% FBS and 0.01% sodium azide) and stained with a cocktail of fluorescently-labeled antibodies targeting surface markers for 30 minutes at room temperature. Antibodies targeting surface markers included mouse anti-human CD14-BV711 (clone M5E2, Biolegend), mouse anti-human CD20-BV711 (clone 2H7, Biolegend), mouse anti-human CD3-APC-Cy7 (clone SP34.2, BD Biosciences), mouse anti-human CD4-BV605 (clone L200, BD Biosciences), mouse anti-human CD8-BUV395 (clone RPA-T8, BD Biosciences), and mouse anti-human CD95-PE-Cy5 (clone DX2, BD Biosciences). Antibody cocktails for surface stain were prepared in FACS buffer supplemented with Brilliant Stain Buffer Plus (BD Biosciences). Following surface stain incubation, samples were washed with FACS buffer before fixation and permeabilization with BD cytofix/cytoperm (BD Biosciences) at 4° C. for 25 minutes. Cells were washed twice with BD perm/wash buffer (BD Biosciences) and then stained with a cocktail of fluorescently-labeled antibodies intracellular cytokines for 60 minutes at room temperature. Antibodies targeting intracellular cytokines included rat anti-human IL-2-PE (clone MQ1-17H12, BD Biosciences), mouse anti-human TNF-PE-Cy7 (clone Mab11, BD Biosciences), rat anti-human IL-4-PE-CF594 (clone MP4-25D2, BD Biosciences), rat anti-human IL-5-Vio515 (clone JES1-39D10, Miltenyi Biotec), and mouse anti-human IFNg-R718 (clone B27, BD Biosciences). Antibody cocktails for intracellular cytokine stain were prepared in BD perm/wash buffer supplemented with Brilliant Stain Buffer Plus. Following incubation, samples were washed with BD perm/wash buffer and fixed with BD stabilizing fixative (BD Biosciences) at 4° C. overnight before sample acquisition on a flow cytometer.

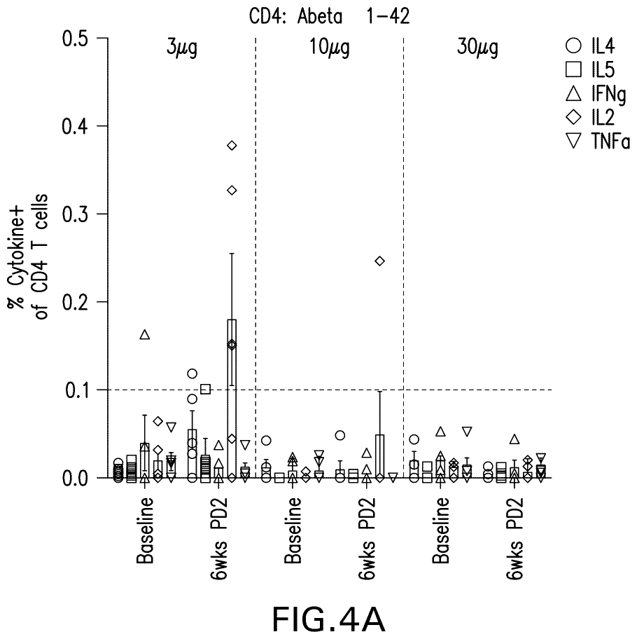

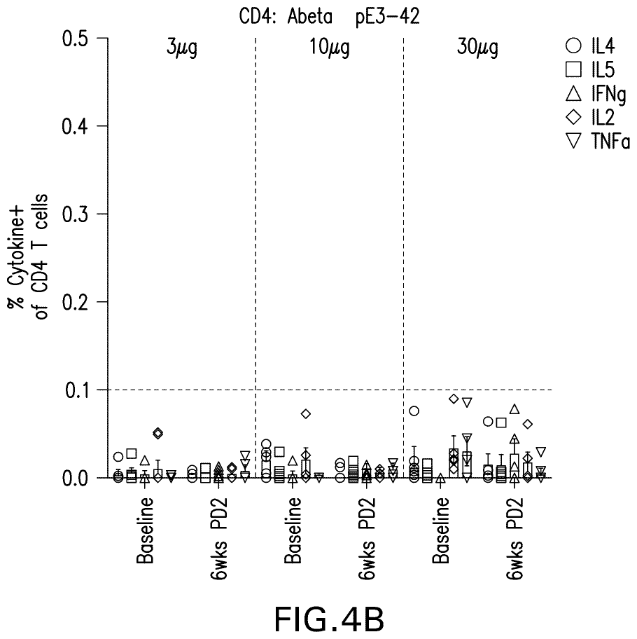

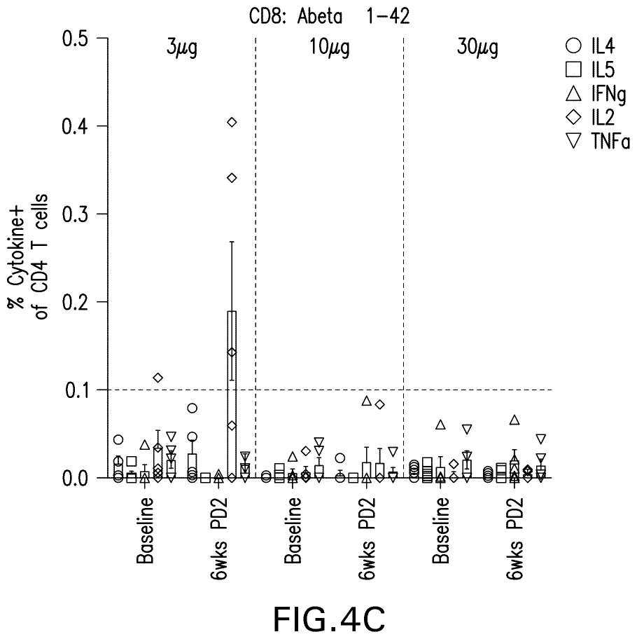

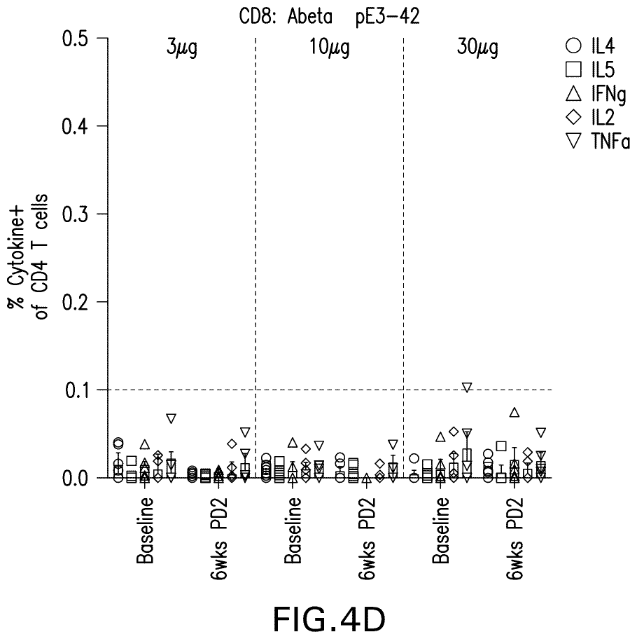

All samples were acquired on a Symphony A5 flow cytometer (BD Biosciences) using FACSDiva (BD Biosciences) software. Data analysis was performed using OMIQ (Dotmatics) and custom R scripts using RStudio (Posit). Data cleaning prior to compensation, scaling, and gating was performed using the OMIQ implementation of flowAI. The percent of cells specifically responding to peptide stimulation was calculated by subtracting the frequency of cells expressing each cytokine in the mock stimulated samples from those stimulated with peptide pools against Aβ₁₋₄₂, Aβ_{pE3-42}, and CRM197. The data, shown graphically in FIGS. 4A-4D, demonstrated that no significant T cell responses were observed in cells isolated from any vaccinated animals.

### Example 11: Mouse Splenocyte Restimulation Assay and Intracellular Cytokine Staining

To determine the magnitude of T cell responses to the test vaccine materials, spleens from vaccinated mice were harvested, processed to single cell suspension, and restimulated with peptide pools targeting Aβ₁₋₄₂, Aβ_{pE3-42}, and CRM197. Briefly, spleens and R10 medium were placed in 60 mm tissue culture dishes containing steel mesh circles (S-3770, Sigma) and mechanically dissociated using the plunger of a sterile 10 ml syringe (BD Biosciences). Cell suspensions were collected into a 15 ml conical, centrifuged to pellet cells, then resuspended in ACK lysis buffer (Gibco) to lyse red blood cells. Reaction was quenched with R10 medium then and cells were washed with R10 medium for a total of two washes. Cell suspensions were resuspended in a final volume of 2 ml and filtered over a 70 μM cell filter (Cellart) to yield final single cell splenocyte suspension. Cells were counted and distributed into 96-well round bottom plates and cultured with peptides spanning the mouse Aβ₁₋₄₂ sequence (15 mers, overlapping by 14 amino acids), mouse Aβ_{pE3-42} sequence (15 mers, overlapping by 14 amino acids), or CRM197 protein (15 mers, overlapping by 11 amino acids) at 2 ug/ml and hamster anti-mouse CD28 (clone 37.51, BD Biosciences) and rat anti-mouse CD49d (clone R1-2, BD Biosciences) costimulatory antibodies at 1.25 ug/ml. Plates were incubated at 37° C. for 60 minutes. Following incubation, freshly diluted brefeldin A was added at a final concentration of 12.5 ug/ml and the plates were incubated for an additional 5 hours. DMSO was added to mock control wells in place of peptide to determine background cytokine expression levels.

Following stimulation, splenocytes were washed with phosphate-buffered saline (PBS) and stained with Live/Dead fixable Aqua stain (Invitrogen) for 15 minutes at room temperature. Samples were washed with FACS buffer (PBS with 1% FBS and 0.01% sodium azide) and stained with mouse Fc block (BD Biosciences) in FACS buffer for 5 minutes at 4° C. Next, a cocktail of fluorescently-labeled antibodies targeting surface markers was added and samples were incubated for 30 minutes at 4° C. Antibodies targeting surface markers included hamster anti-mouse CD3-APC-Cy7 (clone 145-2C11, BD Biosciences), rat anti-mouse CD4-BUV737 (clone RM4-5, BD Biosciences), and rat anti-mouse CD8a-BUV395 (clone 53-6.7, BD Biosciences). Antibody cocktails for surface stain were prepared in FACS buffer supplemented with Brilliant Stain Buffer Plus (BD Biosciences). Following surface stain incubation, samples were washed with FACS buffer before fixation and permeabilization with BD cytofix/cytoperm (BD Biosciences) at 4° C. for 25 minutes. Cells were washed with BD perm/wash buffer (BD Biosciences) and then stained with mouse Fc block (BD Biosciences) in BD perm/wash buffer for 5 minutes at 4° C. Next, a cocktail of fluorescently-labeled antibodies targeting surface markers was added and samples were incubated for 35 minutes at 4° C. Antibodies targeting intracellular cytokines included rat anti-mouse IFNγ-APC (clone XMG1.2, BD Biosciences), rat anti-mouse IL-2-PE-CF594 (clone JES6-5H4, BD Biosciences), rat anti-mouse TNF-PE-Cy7 (clone MP6-XT22, BD Biosciences), rat anti-mouse IL-4-AF488 (clone 11B11, BD Biosciences), rat anti-mouse IL-5-PE (clone TRFK5, BD Biosciences), rat anti-mouse IL-10-BV421 (clone JES5-16E3, BD Biosciences). Antibody cocktails for intracellular cytokine stain were prepared in BD perm/wash buffer supplemented with Brilliant Stain Buffer Plus. Following incubation, samples were washed twice with BD perm/wash buffer and fixed with BD stabilizing fixative (BD Biosciences) at 4° C. overnight before sample acquisition on a flow cytometer.

All samples were acquired on a Symphony A5 flow cytometer (BD Biosciences) using FACSDiva (BD Biosciences) software. Data analysis was performed using OMIQ (Dotmatics) and custom R scripts using Rstudio (Posit). Data cleaning prior to compensation, scaling, and gating was performed using the OMIQ implementation of flowAI. The percent of cells specifically responding to peptide stimulation was calculated by subtracting the frequency of cells expressing each cytokine in the mock stimulated samples from those stimulated with peptide pools against Aβ₁₋₄₂, Aβ_{pE3-42}, and CRM197.

### Example 12: AD Tissue IHC

To demonstrate that vaccine formulations induce antibodies that are relevant to human AD, sera was tested for binding to human AD brain tissue. Serum collected two weeks PD2 from animals treated with pE3-9-G-C-CRM+AVT1 was tested for reactivity with human AD tissue using immunohistochemistry.

Affinity purification of anti-Aβ_{pE3} antibodies from NHP sera. For affinity purification of anti-Aβ^{pE3} antibodies, pyroglutamate Aβ₃₋₄₂ protein (Aβ_{pE3-42}; Anaspec) was coupled to superparamagnetic Dynabeads M280 Tosylactivated (ThermoFisher Scientific) according to manufacturer's instructions. The prepared Aβ_{pE3-42}-beads were incubated with vaccinated NHP serum at room temperature for 30 min with rotation. The bound antibodies were eluted with 0.2 M glycine pH 2.7 and immediately neutralized with 1 M Tris-HCl pH 8.0.

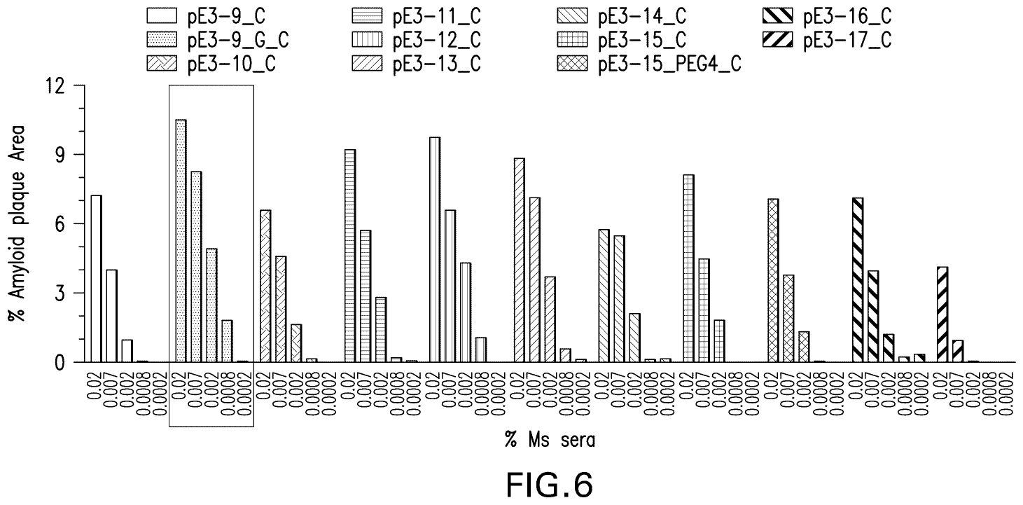

Immunohistochemistry (IHC) and image analysis. Cerebral cortex blocks from individuals who were diagnosed with advanced AD and had a Braak stage of 5 or 6 were purchased from Analytical Biological Services Inc (ABS). These samples were rapidly autopsied within 4 hours of the postmortem interval prior to freezing. To obtain frozen sections, brain tissues were mounted to the cryostat (Thermo Cryostar NX70, CT=−18° C.; OT=−14° C.) and sectioned at 10 m thickness. IHC staining with vaccinated mouse serum or positive control antibody was performed on a Leica Bond RX automated staining instrument using the Bond Polymer Refine Detection System IHC protocol F (DS9800; Leica Biosystems, UK). Briefly, after Protein Block with Background Punisher (BP974M, Biocare Medical) and Peroxide Block treatment, the sections were immunostained with the primary antibody/vaccinated animal serum was diluted in Da Vinci Green Diluent (PD900M, Biocare Medical). 8-point 3× serial dilution of serum, starting from a 500-fold dilution of initial serum or 1 mg/ml antibody, was performed. After washing of primary antibody, Post Primary (rabbit anti mouse IgG in 10% (v/v) animal serum in tris-buffered saline/0.1% ProClin™ 950) was applied. After further rinse, Polymer (anti-rabbit poly-HRP-IgG containing 10% (v/v) animal serum in tris-buffered saline/0.1% ProClin™ 950) was used. The sections were again rinsed, then treated with diaminobenzidine tetrahydrochloride (DAB) and hydrogen peroxide to create a visible reaction product. Hematoxylin was used as nuclear counterstain at the end of staining. Following staining, slides were dehydrated through graded alcohols, mounted with DPX mountant medium (06522; Sigma, USA) and cover-slipped. Slides were imaged using a digital pathology slide scanner-Zeiss Axioscan Z1 (Zeiss) and image analysis performed using the HALO v3.4 software provided by Indica Labs. Experiments were analyzed as a batch. A deep learning algorithm was trained for classification of the images into amyloid plaques and background regions. For this purpose, a representative selection of each class was annotated and then the algorithm (HALO AI DenseNet V2) was trained with these annotations. Grey matter is annotated as region of interest (ROI) for analysis. The image analysis results were exported from HALO as .csv files and analyzed in Pivot Charts/Tables in Excel. The percentage of amyloid plaques were quantified, and statistical analysis was performed using GraphPad Prism 9. As shown in FIG. 6, sera from pE3-9-G_C-CRM197 immunized animals were highly reactive with AD plaques in brain tissue.

Immunohistochemistry (IHC) procedure for NHP sera. Cortex blocks from individuals who were diagnosed with advanced AD and had a Braak stage of 5 or 6 were purchased from PrecisionMed. These samples were rapidly autopsied within 4 hours of the postmortem interval prior to freezing. To obtain frozen sections, brain tissues were mounted to the cryostat (Thermo Cryostar NX70, CT=−18° C.; OT=−14° C.) and sectioned at 10 m thickness. IHC staining with anti-Aβ_{pE3} antibodies was performed on a Leica Bond RX automated staining instrument using the modified Bond Polymer Refine Detection System IHC protocol F (DS9800; Leica Biosystems, UK). Briefly, before the staining, the anti-Aβ_{pE3} antibodies were labeled with digoxigenin using the Human-on-Human HRP-Polymer kit (BRR4056KG; Biocare Medical) according to manufacturer's instructions. After Protein Block with Background Punisher (BP974M; Biocare Medical) and Peroxide Block treatment, the sections were immunostained with the digoxigenin labeled anti-Aβ_{pE3} antibodies diluted in Da Vinci Green Diluent (PD900M; Biocare Medical). 8-point 2× serial dilution of serum, starting from 2 μg/ml antibody, was performed. After washing of primary antibody, Post Primary (mouse anti-digoxigenin secondary (BRR4055G; Biocare Medical)) was applied. After further rinse, Polymer (MACH 2 mouse HRP-polymer (MHRP520G; Biocare Medical)) was used. The sections were again rinsed, then treated with DAB and hydrogen peroxide to create a visible reaction product. Hematoxylin was used as nuclear counterstain at the end of staining. Following staining, slides were dehydrated through graded alcohols, mounted with DPX mountant medium (06522; Sigma, USA) and coverslipped. Slides were imaged using a digital pathology slide scanner-Zeiss Axioscan Z1 and image analysis was performed using the HALO v3.6 software provided by Indica Labs. Experiments were analyzed as a batch. A deep learning algorithm was trained for classification of the images into amyloid plaques and background regions. For this purpose, a representative selection of each class was annotated and the algorithm (HALO AI DenseNet V2) was trained with these annotations. Grey matter was annotated as region of interest (ROI) for analysis. The image analysis results were exported from HALO as .csv files and analyzed in Pivot Charts/Tables in Excel. The percentage of amyloid plaques was quantified and results are shown in Table 13 below. Statistical analysis was performed using GraphPad Prism 9.

TABLE 13
Summary of native plaque binding profiles

| | | | | | AD Donor ID | | AD Donor ID | |
|---|---|---|---|---|---|---|---|---|
| | | | | | Precision Med #5945 | | Precision Med #5945 | |
| | | | | | Day 42 | | Day 70 | |
| | | Peptide | | | Bmax | Peptide | Bmax | |
| | Peptide | Antigen | | | (% Positive | Kd_app | (% Positive | Kd_app |
| Group | Antigen | Dose (ug) | Carrier | Adjuvant | Area) | (nM) | Area) | (nM) |
| A | pE3-9_G_C | 2.5 | CRM197 | MAA | 7.676 | 2.645 | | |
| B | pE3-9_G_C | 2.5 | SH-OMPC | MAA | 7.628 | 2.768 | | |
| C | pE3-9_G_C | 0.5 | SH-OMPC | MAA | 6.206 | 4.185 | | |
| D | pE3-9_G_C | 0.1 | SH-OMPC | MAA | 1.973 | 11.07 | | |
| E | pE3-9_G_C | 2.5 | CRM197 | MAA + AVT8 | 8.932 | 2.404 | 9.832 | 3.755 |
| F | pE3-9_G_C | 2.5 | CRM197 | AVT2 | 9.008 | 2.224 | | |
| G | pE3-9_G_C | 2.5 | CRM197 | MAA + AVT5 | 8.681 | 1.482 | 11.21 | 5.032 |
| H | pE3-9_G_C | 0.5 | SH-OMPC | MAA + AVT4 | 8.825 | 4.682 | | |
| I | pE3-9_G_C | 0.5 | SH-OMPC | AVT2 | 11.75 | 1.901 | 8.671 | 4.053 |
| J | pE3-9_G_C | 0.5 | SH-OMPC | MAA + AVT5 | 10.06 | 2.591 | 7.222 | 5.471 |

### Example 13: Microglia Phagocytosis

The functional activity of sera antibodies induced by the vaccine was further assessed using mouse and human microglia phagocytosis assays.

Mouse microglia phagocytosis assay. A mouse microglia phagocytosis assay was conducted to assess the functional activity of the Aβ vaccine candidates. Postnatal day 2 mouse microglia were obtained from Transnetyx tissue (C57PMWB). The cells were plated at a density of 10,000 cells per well in a PDL coated 384 well plate (Perkin Elmer, PEMSD-6057500) using a liquid handler in the NB Micropro media (NBMicroPro500). The media was changed every 2-3 days by removing 50% of the growth medium and adding an equal volume of fresh media. The cells were allowed to proliferate for 4-5 days and the experiment was conducted within a week of plating.

β-amyloid (pE3-42) peptide (Anaspec; AS-29907) was reconstituted with 100 μl 1% NH4OH (Anaspec; AS-61322) and diluted to 0.1 mg/ml by adding additional 900 μl of a PBS buffer. The solution was warmed for 10-15 mins at 37° C. water bath to ensure it dissolves completely, aliquoted, and then stored at −20° C. as the 2 μM stock. On the day of the assay, 2 μM stock solution was diluted to 0.5 mM Aβ_{pE3-42} in NB Micropro media and was added to a master block plate (Griener; 781270). The vaccine sera samples were pooled for each test group and added starting at 2% (final) concentration and a 2-fold serial dilution was conducted to achieve a 10-point curve. This sera and peptide complex was incubated at 37° C. for 1 hour. The microglia media was then replaced with the peptide and vaccine sera complex, and the cells were incubated at 37° C. for 1 hour. Following this, the cells were then washed with PBS and fixed with 4% paraformaldehyde (Electron Microscopy Sciences; 1224SK) for 15 mins. The cells were then washed with PBS and immunocytochemistry was performed on them.

The plate was blocked with 10% normal goat serum (Sigma; G9023) and 0.3% Triton-x-100 (Sigma; T8787) in PBS along for 1 hour. Following that, the cells were incubated overnight with the primary antibodies rabbit Iba-1 (Wako; 019-19741; 1:500) and mouse 6E10 antibody (Biolegend; 803003; 1:1000) in 5% NGS in PBS at 4° C. The following day, the cells were washed three times with PBS, for 5 mins each. The secondary antibodies Alexafluor secondary antibody rabbit 488 (Molecular probes; A32731; 1:1000) and mouse 555 (Molecular probes; A32727, 1:1000) were then added in 5% BSA, 0.3% T-x-100/PBS for 1 hour at room temperature. The cells were then washed with PBS two times and incubated with Hoesct (Anaspec; 83218; 1:5000) for 5 mins. The cells were given a final wash with PBS and then imaged on the Operetta CLS High Content plate imager. Multiple fields (15-20) per well and n=3 per treatment group was imaged for vaccine sera assessment.

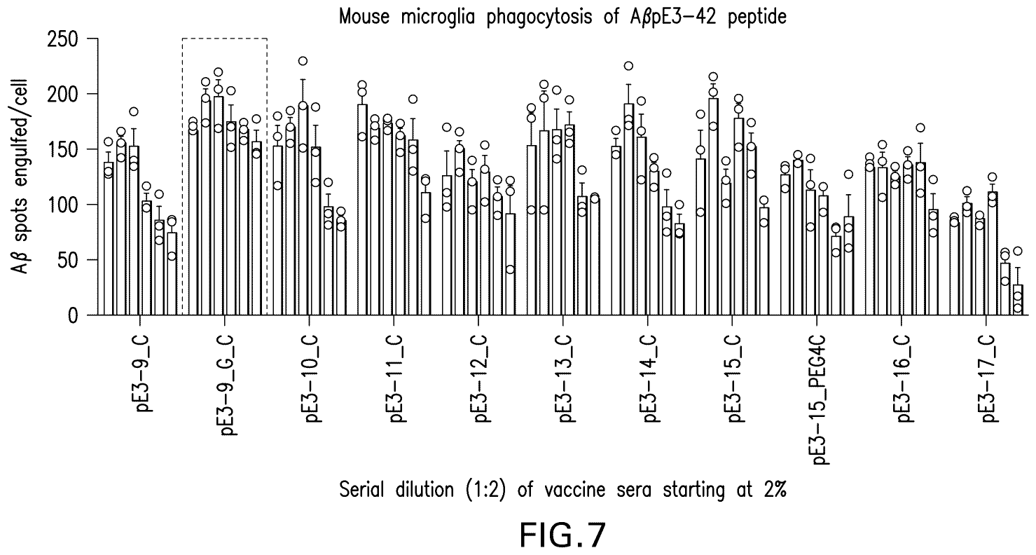

The images were then analyzed using the built-in Operetta module to assess the number of Aβ_{pE3-42} spots engulfed per microglial cell. This data was then exported to conduct statistical analysis of the final data using GraphPad prism software. As shown in FIG. 7, the pE_3-9_G_C-CRM197 vaccinated group showed the most robust level of phagocytosis, even at lower dilution of sera.

Induced human microglia (iMGL) phagocytosis assay. A human phagocytosis assay was conducted to assess the functional activity of the different adjuvant non-human primate sera at day 42 using human microglia derived from human induced pluripotent stem cells (iPSCs). Non-human primate sera are shown in Table 14 below.

TABLE 14
Non-human primate sera

| Group | Peptide Antigen | Carrier | Adjuvant |
|---|---|---|---|
| A | pE3-9_G_C | CRM197 | MAA |
| B | pE3-9_G_C | SH-OMPC (2.5) | MAA |
| C | pE3-9_G_C | SH-OMPC (0.5) | MAA |
| D | pE3-9_G_C | SH-OMPC (0.1) | MAA |
| E | pE3-9_G_C | CRM197 | MAA + AVT8 |
| F | pE3-9_G_C | CRM197 | AVT2 |
| G | pE3-9_G_C | CRM197 | MAA + AVT5 |
| H | pE3-9_G_C | SH-OMPC | MAA + AVT4 |
| I | pE3-9_G_C | SH-OMPC | AVT2 |
| J | pE3-9_G_C | SH-OMPC | MAA + AVT5 |

The human BX-0900-CS-2M iPS cell line (BrainXell) was cultured and induced to differentiate into human microglial cells (iMGLs) according to supplier protocols. Reagents and media formulations for the culture and differentiation of BX-0900-CS-2M are shown in Tables 15 and 16 below.

TABLE 15
Reagent list

| Reagent | Vendor | Catalogue # |
|---|---|---|
| Human Microglia Differentiation from | BrainXell | BX-0900-CS- |
| iPSCs, | | 2M |
| BD Matrigel Matrix Growth Factor | Corning | 356231 |
| Reduced, Phenol Red-Free | | |
| DMEM/F-12, HEPES, no phenol red | ThermoFisher | 11039021 |
| Advanced DMEM/F12 | ThermoFisher | 12634010 |
| B27 supplement | ThermoFisher | 17504044 |
| BSA solution | Militenyi | 130-091-376 |
| N2 supplement | ThermoFisher | 17502048 |
| Non-essential amino acids (NEAA) | ThermoFisher | 11140050 |
| GlutaMax supplement | ThermoFisher | 35050061 |
| Human insulin | Sigma | I2643-50MG |
| Insulin-Transferrin-Selenium (ITS-G) | ThermoFisher | 41400045 |
| (100X) | | |
| Monothioglycerol | Sigma | M1753-100ML |
| Corning ™ BioCoat ™ 96-Well, | Corning | 08-774-256 |
| Poly-D Lysine-Treated, Flat-Bottom | | |
| Microplate (50) | | |
| Human Amyloid Beta Pyroglutamate | Stress Marq | Catalog No. |
| 3-42 Preformed Fibrils Catalog | Biosciences | SPR-492 |
| No. SPR-492 | | |
| 4% paraformaldehyde (PFA), 4% | EMS | 1224-SK |
| sucrose in DPBS | | |
| Corning ™ BioCoat ™ 96-Well, | Corning | 08-774-256 |
| Poly-D Lysine-Treated, Flat-Bottom | | |
| Microplate (50) | | |

TABLE 16
Base and differentiation media

| | Stock | | Final |
|---|---|---|---|
| Reagent | Concentration | Volume | Concentration |
| DMEM/F12 (phenol free) | — | | |

| | | | | |
|---|---|---|---|---|
| Non-essential Amino Acids | 100X | 5 | mL | 1X |
| Glutamax | 100X | 5 | mL | 1X |

Filter Above in 0.22 um Filter (EMD Millipore)

| B27 supplement | 50X | 10 | mL | 1X | |
|---|---|---|---|---|---|
| N2 supplement | 100X | 2.5 | mL | 0.5X | |
| ITSG solution | 100X | 10 | mL | 2X | |
| Monothioglycerol | 1M | 200 | μL | 400 | μM |
| Insulin | 5 mg/mL | 500 | μL | 5 | μg/mL |

Complete differentiation medium: Base medium + M-CSF (25 ng/mL) + IL-34 (100 ng/mL)

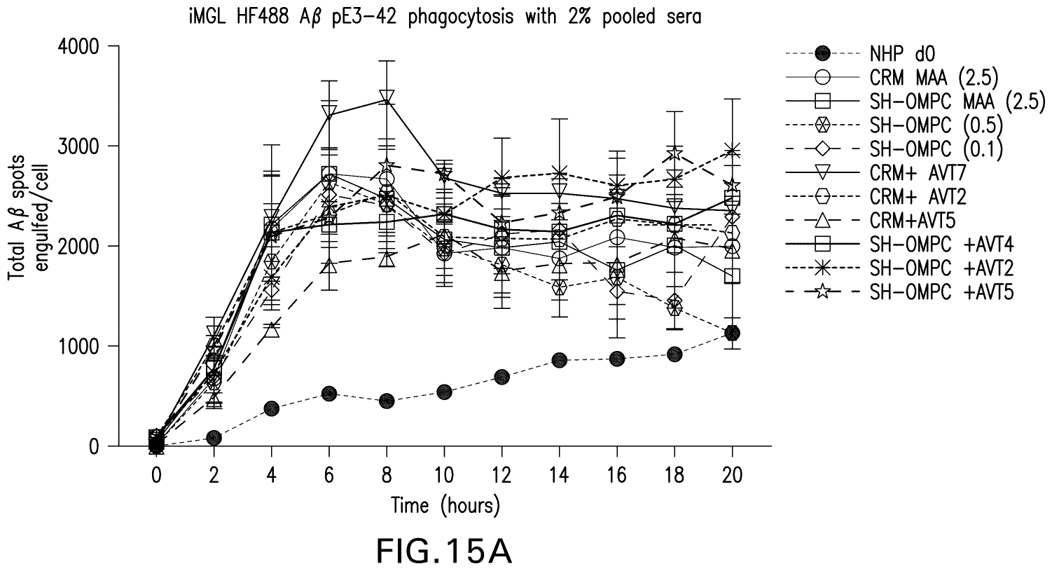

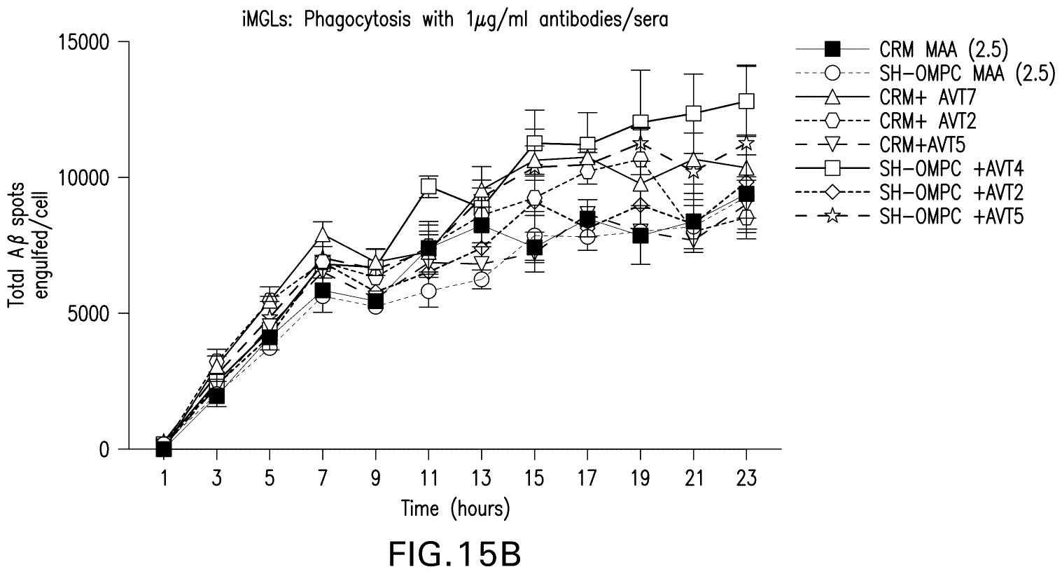

Phagocytosis of HF-488 labeled Aj3 peptide by human iMGLs was observed over 24 hours with live cell imaging. On the day of the experiment, half of the media (50 μl) was removed from the multiwell culture plates and replaced with 2× red cell tracker (Thermofisher; C34552) with a final concentration of 1:4000 final and 1 drop/ml Nucblue (Thermofisher; R37605) and incubated for one hour. During this time, 0. μM of labeled Aβ pyro E3-42 peptide (Anaspec; 83960-3) was prepared in microglia complete differentiation media and incubated in a first plate with 2% NHP sera and second plate with 1 pig/ml of purified NHP day 42 sera for 1 hour at 37° C. Microglial conditioned culture media was replaced with 100 l Aβ peptide and sera or antibody complex and incubate at 37° C. while conducting live imaging of cells for 24 hours. The plates were imaged on the Operetta CSL high content imager at 40× magnification. Multiple fields per well and n=3 per treatment group were imaged for vaccine sera assessment. The images were analyzed using the Harmony software and the number of Aβ spots engulfed per cell was determined as an outcome measure. The data were exported and further analyzed on graph pad prism software. A significant increase in phagocytosis was noted in day 42 sera compared to day 0 sera (FIG. 15A). Similarly, phagocytosis of labeled Aβ_{pE3-42} upon addition of purified NHP sera at day 42 at the concentration of 1 μg/ml was added for all group and imaged over 24 hours (FIG. 15B). The data are plotted as number of engulfed Aβ_{pE3-42} spots per cells (Y axis) for each of the treatment groups over time in hours (X axis).

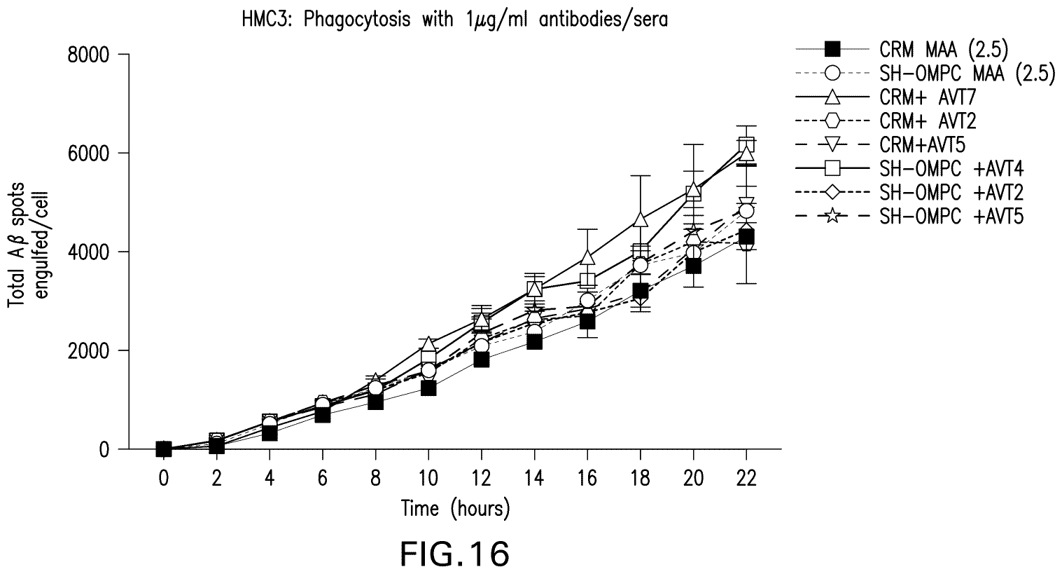

HMC3 human microglia phagocytosis assay. A human phagocytosis assay was conducted with the human microglial cell line HMC3 to assess the functional activity of the different adjuvant non-human primate sera at day 42. HMC3 cells were spun down at 280 g for 5 min at room temperature (RT), washed, and resuspended in maxcyte electroporation buffer to a final cell density of 1×10⁸ cells/ml. The resuspended cells were electroporated with 2 μl of 1 μg/μl FCGR1A DNA (100 μl of aliquoted cells, 2 μg per 10⁶ cells). Electroporated cells were transferred to warm media and plated in 96 well culture plates at a density of 1:5000 cells/well. Plated cells were incubated at 37° C., 5% CO2 and high humidity, for 24 hours. On the day of the experiment, half of the media was removed (50 μl) and replaced with 2× red cell tracker with a final concentration of 1:4000 final and Nucblue (1 drop/ml) and incubated for one hour. During this time, 0.5 μM of the labeled Aβ pyro E3-42 peptide was prepared in HMC3 media and incubated with 1 μg/ml of purified NHP day 42 sera for 1 hour at 37° C. Microglial conditioned culture media was replaced with 100 μl Aβ peptide and sera or antibody complex and incubate at 37° C. while conducting live imaging of cells for 24 hours. The cells were live imaged for Abeta HF488 with cell tracker at 20× and analyzed using Harmony software to examine the number of spots engulfed. The cells were then fixed using 4% PFA paraformaldehyde (Electron Microscopy Sciences; 1224SK) for 15 mins. The plates were imaged on the Operetta CSL high content imager at 40× magnification. Multiple fields per well and n=3 per treatment group were imaged for vaccine sera assessment. The images are analyzed using the Harmony software and the number of Aβ spots engulfed per cell was measured as an outcome. The data was exported and further analyzed on graph pad prism software. The data were plotted as the number of engulfed Aβ_{pE3-42} spots per cells (Y axis) for each of the treatment groups over time in hours (X axis) (FIG. 16).

### Example 14: Analysis of Antigen-CRM197 Linkers

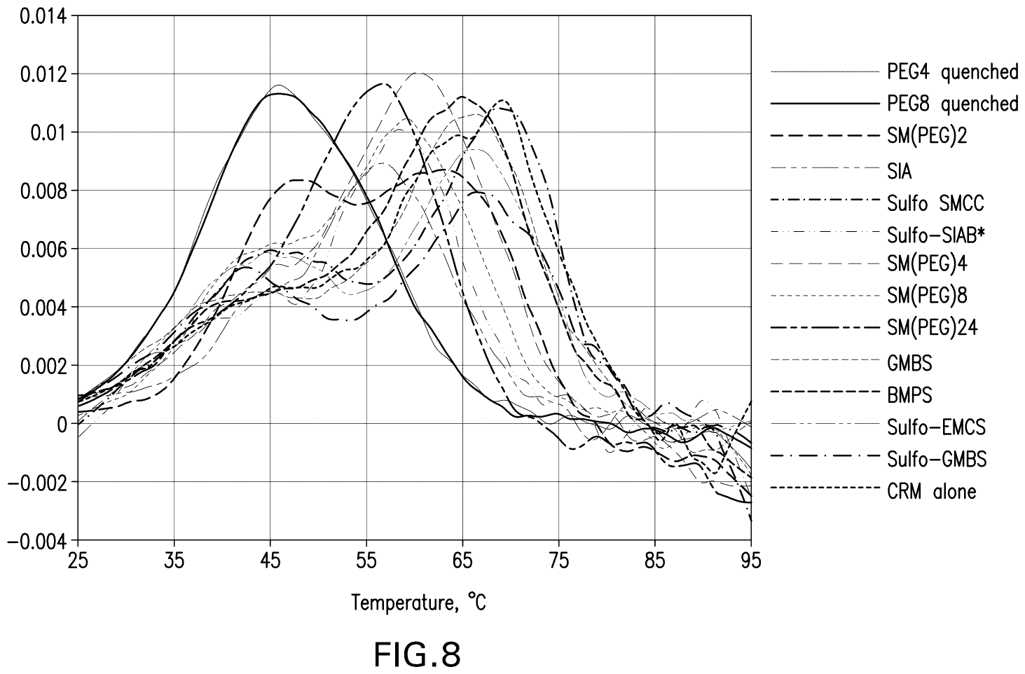

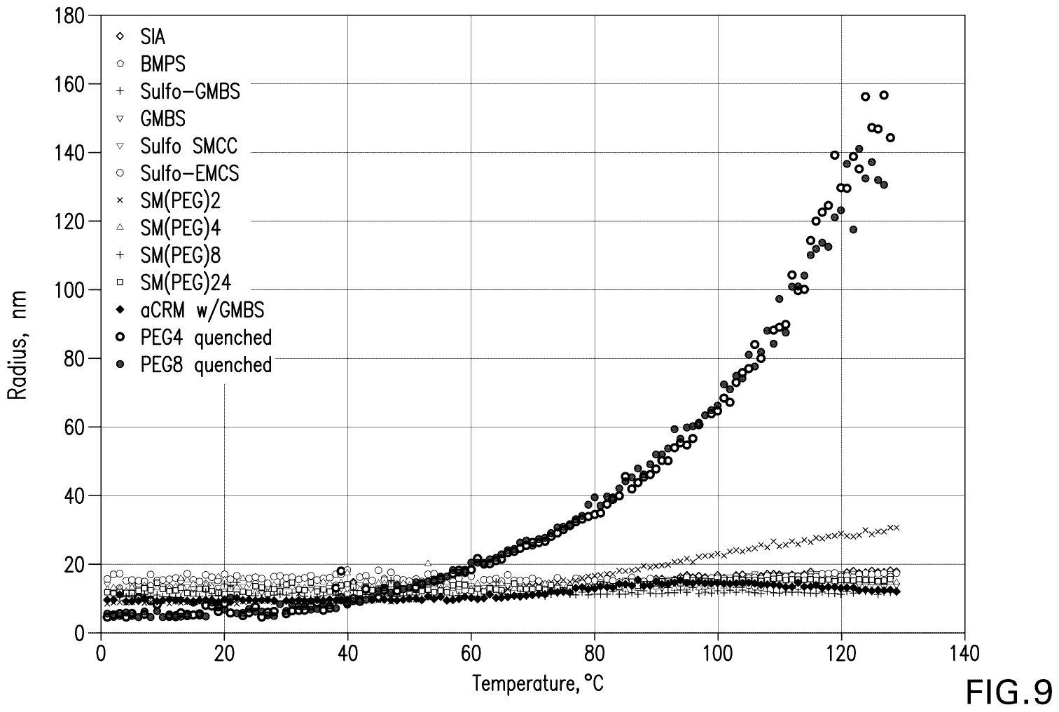

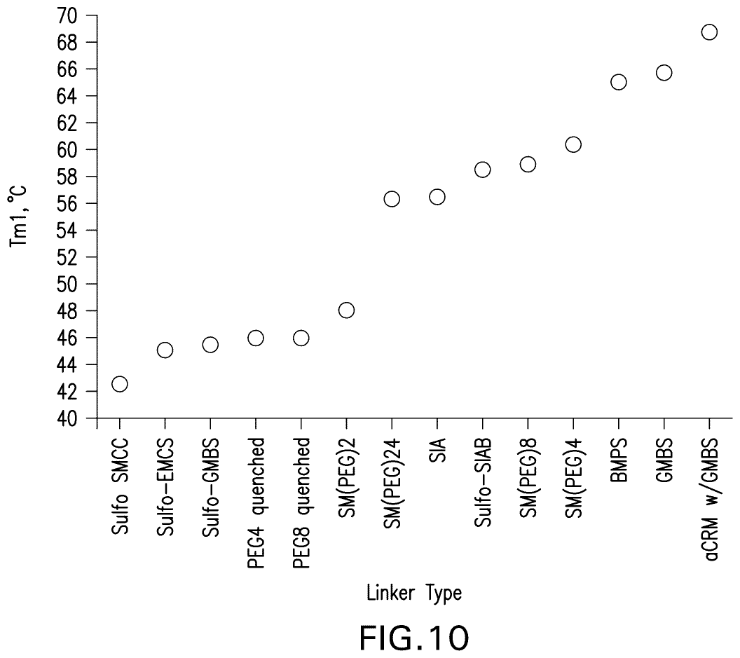

pE3-9-CRM197 samples comprising different linkers were subjected to various analyses to evaluate their physical stability. The samples were prepared in a 364 well plate and their thermal stability was assessed using a Prometheus NanoTemper differential scanning fluorimetry (nanoDSF) instrument. In nanoDSF, a protein in solution was exposed to a temperature gradient that led to the unfolding of the protein. The intrinsic fluorescence of the protein, mainly originating from the aromatic sidechains of tyrosine and tryptophan residues, was examined and the data are shown in FIG. 8. In addition, the melting temperature of the pE3-9-CRM197 conjugates and the first derivative of fluorescent signal at 350 nm versus the melt temperature for each linker type were determined, as shown in FIG. 9 and FIG. 10, respectively.

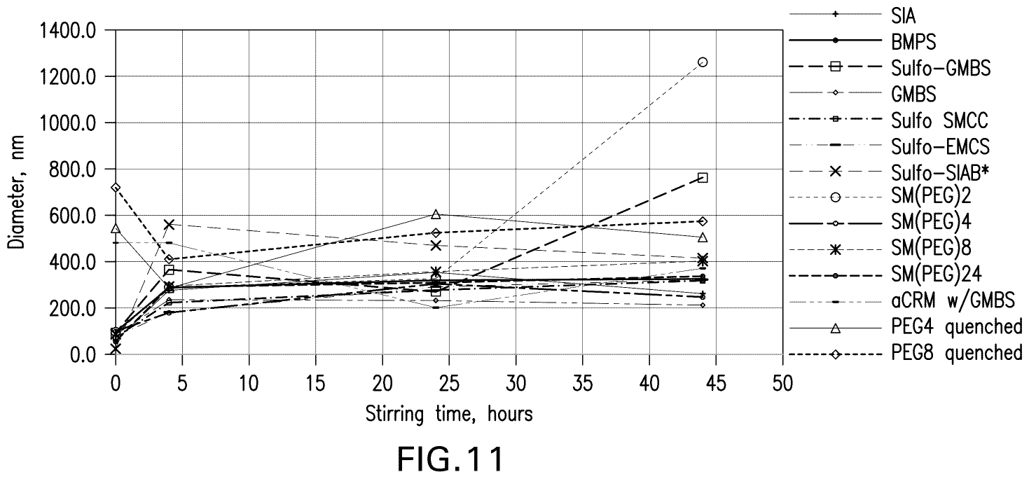

Furthermore, the linkers were examined for their potential to cause aggregation in a tumble stir stressed experiment. To perform the experiments, the samples were diluted to a concentration of 0.18 mg/mL in a buffer containing 25 mM HEPES, 150 mM NaCl, and pH 7.4. The diluted samples were then placed on a magnetic tumble stir plate set to 60% power. At specific time intervals (0, 4, 6, and 24 hours), aliquots were taken and transferred to a 96 well plate for dynamic light scattering analysis, which measures the intensity of scattered light to determine particle size and aggregation status. These data are shown in FIG. 11.

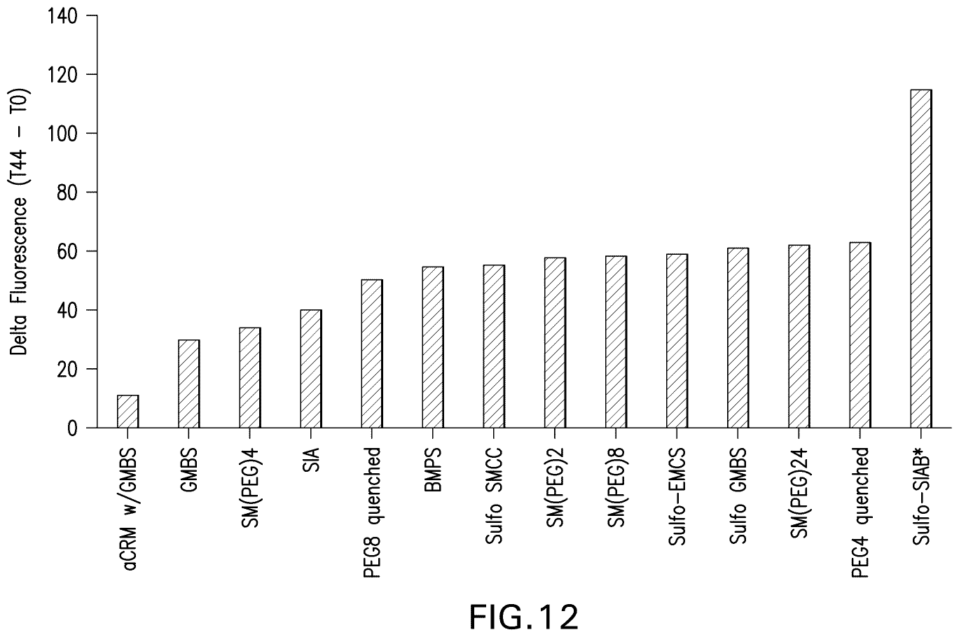

Additionally, stressed samples were plated in a black-walled 96 well plate and treated with PROTEOSTAT dye. This dye selectively binds to aggregated proteins and can be used as an indicator of protein aggregation. The fluorescence was measured on a SpectraMax M5 plate reader, with excitation and emission set to 550 nm and 600 nm, respectively. These data are shown in FIG. 12.

A similar assay was leveraged to assess the impact of polysorbate 80 and sucrose on formulation stability. To perform these experiments, the samples were diluted to a concentration of 0.18 mg/mL in a buffer containing 25 mM HEPES, 150 mM NaCl, and pH 7.4, and 0, 0.01, 0.02, 0.05, 0.1 or 0.2% PS80. These samples were then placed on a magnetic tumble stir plate set to 60% power. At specific time intervals (0, 4, 6, and 24 hours), aliquots were taken and transferred to a 96 well plate for dynamic light scattering analysis, which measures the intensity of scattered light to determine particle size and aggregation status.

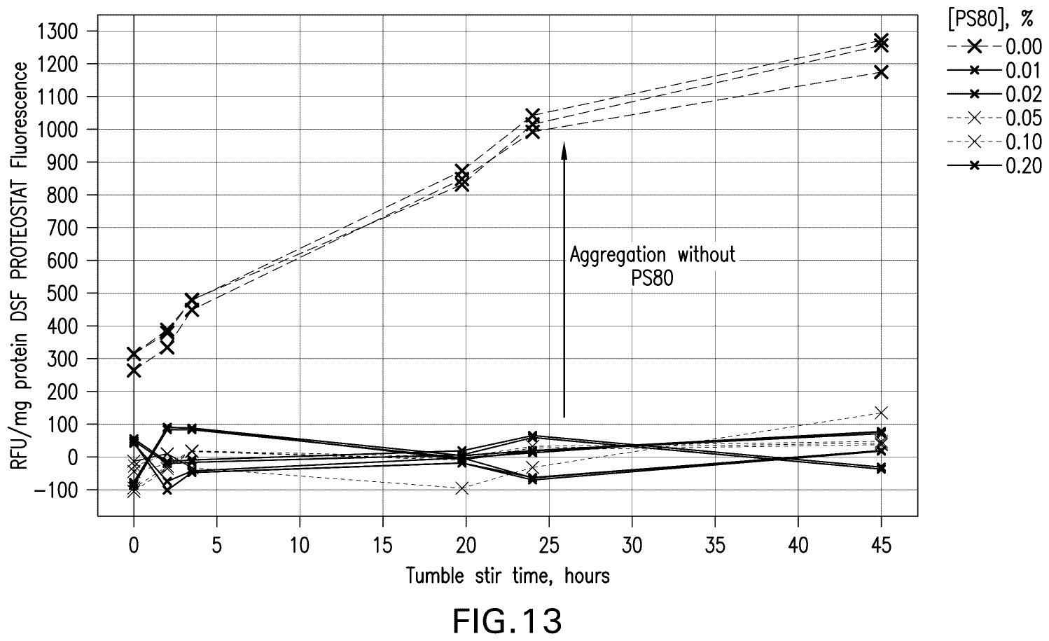

Stressed samples were also plated in a black-walled 96 well plate and treated with PROTEOSTAT dye. This dye selectively binds to aggregated proteins and can be used as an indicator of protein aggregation. The fluorescence was measured on a SpectraMax M5 plate reader, with excitation and emission set to 550 nm and 600 nm, respectively. These data, shown in FIG. 13, demonstrate that pE3-9_G_C-CRM197 formulated with polysorbate 80 inhibits aggregation.

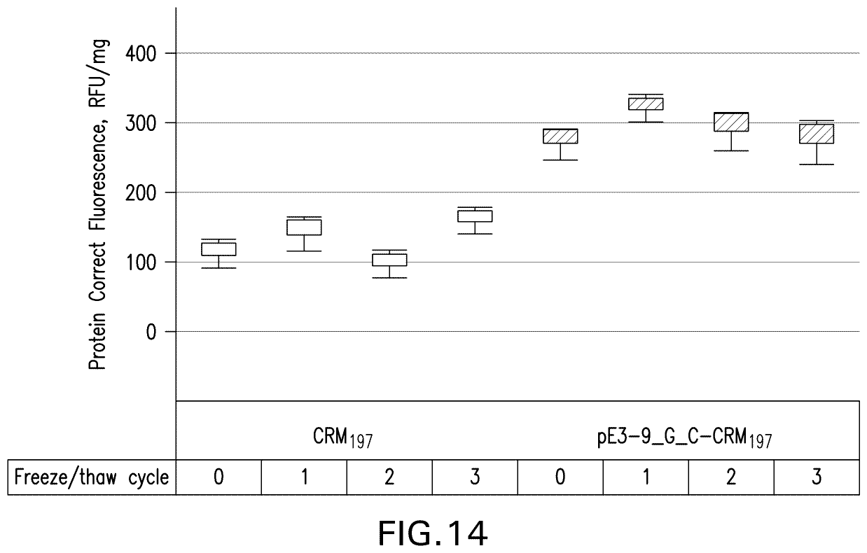

A similar assay was leveraged to assess the impact of sucrose on formulation stability. To perform this experiments, the samples were diluted to a concentration of 0.18 mg/mL in a buffer containing 25 mM HEPES, 150 mM NaCl, 10% sucrose, pH 7.4. Samples underwent 0, 1, 2, or 3 freeze thaw cycles. After each freeze thaw cycle, the sample was plated in a 96-well plate with PROTEOSTAT dye. These data, shown in FIG. 14.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A pharmaceutical composition comprising an immunogenic peptide of at least six contiguous amino acids of SEQ ID NO:1 and an immunogenic carrier protein.

2. The pharmaceutical composition of claim 1, wherein the immunogenic peptide lacks 1-10 amino acids at the N-terminus and/or 1-33 amino acids at the C-terminus of SEQ ID NO:1.

3. (canceled)

4. The pharmaceutical composition of claim 1, wherein the immunogenic peptide comprises at least ten contiguous amino acids of SEQ ID NO:1, optionally wherein the immunogenic peptide comprises 1-5 modified amino acids, optionally wherein the immunogenic peptide comprises a pyroglutamate residue at amino acid position 3 and/or 11 of SEQ ID NO:1, and optionally wherein the immunogenic peptide is selected from the group consisting of SEQ ID NOs: 2-13.

5-7. (canceled)

8. The pharmaceutical composition of claim 1, wherein the immunogenic carrier protein is selected from the group consisting of: CRM197; diphtheria toxin fragment B (DTFB); DTFB C8; diphtheria toxoid (DT); tetanus toxoid (TT); fragment C of TT; pertussis toxoid; cholera toxoid; E. coli LT; E. coli ST; Neisseria meningitidis outer membrane protein complex (OMPC); exotoxin A from Pseudomonas aeruginosa; mariculture keyhole limpet hemocyanin (mcKLH); and bacteriophage AP205 coat protein, optionally wherein the immunogenic carrier protein is conjugated at the N-terminus or C-terminus of the immunogenic peptide, optionally wherein the immunogenic carrier protein is conjugated to the immunogenic peptide with a linker, and optionally wherein the linker is selected from the group consisting of: N-γ-maleimidobutyryl-oxysuccinimide ester (GMBS); polyethylene glycol (PEG); aminohexanoic acid (Ahx); a sulfydryl-reactive crosslinker; a maleimide (MA) linker; an oligopeptide; a dendrimer; cyclodextrine; and a glycine-rich peptide.

9-12. (canceled)

13. The pharmaceutical composition of claim 8 further comprising a spacer comprising 1-10 amino acids adjacent to the linker, optionally wherein the immunogenic peptide comprises SEQ ID NO:2 and the immunogenic carrier protein is CRM197, and optionally wherein the immunogenic peptide is monomeric or multimeric.

14. (canceled)

15. The pharmaceutical composition of claim 13, wherein the immunogenic peptide is linked to CRM197 through a GMBS linker, and wherein the composition further comprises a glycine-cysteine spacer between the immunogenic peptide and the GMBS linker.

16-17. (canceled)

18. The pharmaceutical composition of claim 1, further comprising a pharmaceutically acceptable adjuvant, and optionally wherein the pharmaceutically acceptable adjuvant is selected from the group consisting of: glucopyranosol lipid adjuvant (GLA); AVT1; AVT2; AVT3, AVT4; AVT5; AVT6; AVT7; QS-21; an aluminum-based adjuvant; a saponin-based adjuvant; and a TLR7/8 agonist.

19-20. (canceled)

21. A method for preventing or treating a disease associated with amyloid deposits of Aβ in the brain of a patient in need thereof, comprising administering an effective dose of a pharmaceutical composition comprising an immunogenic peptide of at least six contiguous amino acids of SEQ ID NO:1 and an immunogenic carrier protein.

22. The method of claim 21, wherein the immunogenic peptide lacks 1-10 amino acids at the N-terminus and/or 1-33 amino acids at the C-terminus of SEQ ID NO:1.

23. (canceled)

24. The method of claim 21, wherein the immunogenic peptide comprises at least ten contiguous amino acids of SEQ ID NO:1, optionally wherein the immunogenic peptide comprises 1-5 modified amino acids, optionally wherein the immunogenic peptide comprises a pyroglutamate residue at amino acid position 3 and/or 11 of SEQ ID NO:1, and optionally wherein the immunogenic peptide is selected from the group consisting of SEQ ID NOs: 2-13.

25-27. (canceled)

28. The method of claim 21, wherein the immunogenic carrier protein is selected from the group consisting of: CRM197; diphtheria toxin fragment B (DTFB); DTFB C8; diphtheria toxoid (DT); tetanus toxoid (TT); fragment C of TT; pertussis toxoid; cholera toxoid; E. coli LT; E. coli ST; Neisseria meningitidis outer membrane protein complex (OMPC); exotoxin A from Pseudomonas aeruginosa; mariculture keyhole limpet hemocyanin (mcKLH); and bacteriophage AP205 coat protein, optionally wherein the immunogenic carrier protein is conjugated at the N-terminus or C-terminus of the immunogenic peptide, optionally wherein the immunogenic carrier protein is conjugated to the immunogenic peptide with a linker, and optionally wherein the linker is selected from the group consisting of: N-γ-maleimidobutyryl-oxysuccinimide ester (GMBS); polyethylene glycol (PEG); aminohexanoic acid (Ahx); a sulfydryl-reactive crosslinker; a maleimide (MA) linker; an oligopeptide; a dendrimer; cyclodextrine; and a glycine-rich peptide.

29-32. (canceled)

33. The method of claim 31, further comprising a spacer comprising 1-10 amino acids adjacent to the linker, optionally wherein the immunogenic peptide comprises SEQ ID NO:2 and the immunogenic carrier protein is CRM197, and optionally wherein the immunogenic peptide is monomeric or multimeric.

34. (canceled)

35. The method of claim 33, wherein the immunogenic peptide is linked to CRM197 through a GMBS linker, and wherein the composition further comprises a glycine-cysteine spacer between the immunogenic peptide and the GMBS linker.

36-37. (canceled)

38. The method of claim 21, further comprising a pharmaceutically acceptable adjuvant, and optionally wherein the pharmaceutically acceptable adjuvant is selected from the group consisting of: glucopyranosol lipid adjuvant (GLA); AVT1; AVT2; AVT3, AVT4; AVT5; AVT6; AVT7; QS-21; an aluminum-based adjuvant; a saponin-based adjuvant; and a TLR7/8 agonist.

39-40. (canceled)

41. The method of claim 21, wherein the disease associated with amyloid deposits of Aβ in the brain is a disease selected from the group consisting of: Alzheimer's Disease (AD); cerebral amyloid angiopathy (CAA); inflammatory CAA; and cerebral amyloidoma.

42. (canceled)

43. Use of the pharmaceutical composition of claim 1 in the manufacture of a medicament for preventing or treating a disease associated with amyloid deposits of A in the brain of a patient in need thereof.

44. The use of claim 43, wherein the disease associated with amyloid deposits of AR in the brain is a disease selected from the group consisting of: Alzheimer's Disease (AD); cerebral amyloid angiopathy (CAA); inflammatory CAA; and cerebral amyloidoma.

45. (canceled)

46. A method of inducing an immune response to an Aβ peptide in a patient in need thereof, comprising administering to the patient an immunologically effective dose of the pharmaceutical composition of claim 1.

47. The method of claim 46, wherein the disease associated with amyloid deposits of Aβ in the brain is a disease selected from the group consisting of: AD; CAA; inflammatory CAA; and cerebral amyloidoma.

48. (canceled)


SEQUENCE LISTING (43 sequences)

SEQ ID NO: 1 - protein, 42 aa, synthetic construct [sequence omitted]

SEQ ID NO: 2 - protein, 7 aa, synthetic construct
  SITE 1: note = Pyroglutamate
XFRHDSG

SEQ ID NO: 3 - protein, 9 aa, synthetic construct
  SITE 1: note = Pyroglutamate
XFRHDSGGC

SEQ ID NO: 4 - protein, 8 aa, synthetic construct
  SITE 1: note = Pyroglutamate
XFRHDSGY

SEQ ID NO: 5 - protein, 9 aa, synthetic construct
  SITE 1: note = Pyroglutamate
XFRHDSGYE

SEQ ID NO: 6 - protein, 10 aa, synthetic construct
  SITE 1: note = Pyroglutamate
XFRHDSGYEV

SEQ ID NO: 7 - protein, 11 aa, synthetic construct
  SITE 1: note = Pyroglutamate
XFRHDSGYEVH

SEQ ID NO: 8 - protein, 12 aa, synthetic construct
  SITE 1: note = Pyroglutamate
XFRHDSGYEVHH

SEQ ID NO: 9 - protein, 13 aa, synthetic construct
  SITE 1: note = Pyroglutamate
XFRHDSGYEVHHQ

SEQ ID NO: 10 - protein, 14 aa, synthetic construct
  SITE 1: note = Pyroglutamate
XFRHDSGYEVHHQK

SEQ ID NO: 11 - protein, 15 aa, synthetic construct
  SITE 1: note = Pyroglutamate
XFRHDSGYEVHHQKL

SEQ ID NO: 12 - protein, 7 aa, synthetic construct
  SITE 1: note = Pyroglutamate
XVHHQKL

SEQ ID NO: 13 - protein, 9 aa, synthetic construct
  SITE 1: note = Pyroglutamate
XVHHQKLGC

SEQ ID NO: 14 - protein, 9 aa, synthetic construct
  SITE 1: note = Pyroglutamate
  SITE 8: note = Aminohexanoic acid
XFRHDSGXC

SEQ ID NO: 15 - protein, 9 aa, synthetic construct
  SITE 1: note = Pyroglutamate
XFRHDSGGC

SEQ ID NO: 16 - protein, 10 aa, synthetic construct
  SITE 1: note = Pyroglutamate
  SITE 9: note = Aminohexanoic acid
XFRHDSGYXC

SEQ ID NO: 17 - protein, 11 aa, synthetic construct
  SITE 1: note = Pyroglutamate
  SITE 10: note = Aminohexanoic acid
XFRHDSGYEXC

SEQ ID NO: 18 - protein, 12 aa, synthetic construct
  SITE 1: note = Pyroglutamate
  SITE 11: note = Aminohexanoic acid
XFRHDSGYEVXC

SEQ ID NO: 19 - protein, 13 aa, synthetic construct
  SITE 1: note = Pyroglutamate
  SITE 12: note = Aminohexanoic acid
XFRHDSGYEVHXC

SEQ ID NO: 20 - protein, 14 aa, synthetic construct
  SITE 1: note = Pyroglutamate
  SITE 13: note = Aminohexanoic acid
XFRHDSGYEVHHXC

SEQ ID NO: 21 - protein, 15 aa, synthetic construct
  SITE 1: note = Pyroglutamate
  SITE 14: note = Aminohexanoic acid
XFRHDSGYEVHHQXC

SEQ ID NO: 22 - protein, 13 aa, synthetic construct
  SITE 1: note = Pyroglutamate
XFRHDSGYEVHHQ

SEQ ID NO: 23 - protein, 16 aa, synthetic construct
  SITE 1: note = Pyroglutamate
  SITE 15: note = Aminohexanoic acid
XFRHDSGYEVHHQKXC

SEQ ID NO: 24 - protein, 17 aa, synthetic construct
  SITE 1: note = Pyroglutamate
  SITE 16: note = Aminohexanoic acid
XFRHDSGYEVHHQKLXC

SEQ ID NO: 25 - protein, 9 aa, synthetic construct
  SITE 1: note = Pyroglutamate
  SITE 8: note = Aminohexanoic acid
XVHHQKLXC

SEQ ID NO: 26 - protein, 9 aa, synthetic construct
  SITE 1: note = Pyroglutamate
XVHHQKLGC

SEQ ID NO: 27 - protein, 9 aa, synthetic construct
  SITE 1: note = Pyroglutamate
  SITE 9: note = Bromoacetamide modified residue
XFRHDSGGK

SEQ ID NO: 28 - protein, 14 aa, synthetic construct
  SITE 1: note = Pyroglutamate
  SITE 13: note = Aminohexanoic acid
  SITE 14: note = Bromoacetamide modified residue
XFRHDSGYEVHHXK

SEQ ID NO: 29 - protein, 21 aa, synthetic construct
  SITE 1: note = Pyroglutamate
XFRHDSGGAHIVMVDAYKPTK

SEQ ID NO: 30 - protein, 21 aa, synthetic construct
  SITE 1: note = Pyroglutamate
  SITE 8: note = Aminohexanoic acid
XFRHDSGXAHIVMVDAYKPTK

SEQ ID NO: 31 - protein, 7 aa, synthetic construct
  SITE 1: note = Pyroglutamate
XFRHDSG

SEQ ID NO: 32 - protein, 8 aa, synthetic construct
  SITE 1: note = Pyroglutamate
  SITE 8: note = Amidated residue with side chain attached to another sequence via S-(1-(1-(60-(3-mercapto-2,5-dioxopyrr olidin-1-yl)-45-(15-(3-mercapto-2,5-dioxopyrrolidin-1-yl)- 13-oxo-3,6,9-trioxa-12-azapentadecyl)-15-(44-(3-mercapto-2 ,5-dioxopyrrolidin-1-yl)-29-(15-(3-mercapto-2,5-dioxopyrro lidin-1-yl)-13-oxo-3,6,9-trioxa-12-azapentadecyl)-16,28,42 -trioxo-3,6,9,12,19,22,25,32,35,38-decaoxa-15,29,41-triaza tetratetracontyl)-31,44,58-trioxo-3,6,9,12,18,21,24,27,34, 37,41,48,51,54-tetradecaoxa-15,30,45,57-tetraazahexacontyl )-1H-1,2,3-triazol-4-yl)-2,5,8,11-tetraoxatridecan-13-yl) ethanethioate
XFRHDSGC

SEQ ID NO: 33 - protein, 7 aa, synthetic construct
  SITE 1: note = Pyroglutamate
XFRHDSG

SEQ ID NO: 34 - protein, 7 aa, synthetic construct
  SITE 1: note = Pyroglutamate
XFRHDSG

SEQ ID NO: 35 - protein, 7 aa, synthetic construct
  SITE 1: note = Pyroglutamate
XFRHDSG

SEQ ID NO: 36 - protein, 13 aa, synthetic construct
AHIVMVDAYKPTK

SEQ ID NO: 37 - protein, 116 aa, synthetic construct
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 38 - protein, 21 aa, synthetic construct
  SITE 1: note = Pyroglutamate
XFRHDSGGAHIVMVDAYKPTK

SEQ ID NO: 39 - protein, 21 aa, synthetic construct
  SITE 1: note = Pyroglutamate
  SITE 8: note = Aminohexanoic acid
XFRHDSGXAHIVMVDAYKPTK

SEQ ID NO: 40 - protein, 7 aa, synthetic construct
  SITE 1: note = Pyroglutamate
XFRHDSG

SEQ ID NO: 41 - protein, 13 aa, synthetic construct
AHIVMVDAYKPTK

SEQ ID NO: 42 - protein, 9 aa, synthetic construct
  SITE 1: note = Pyroglutamate
  SITE 9: note = Amidated residue with side chain attached to immunogenic carrier protein CRM197 via a GMBS (N-gamma-maleimidobutyryl-oxysuccinimide ester) linker
XFRHDSGGC

SEQ ID NO: 43 - protein, 9 aa, synthetic construct
  SITE 1: note = Pyroglutamate
  SITE 9: note = Amidated residue
XFRHDSGGC